SEQUENCE LISTING (48 sequences)

SEQ ID NO: 1 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 2 - protein, 21 aa, synthetic construct
IVGGHEAQPHSRPYMASLQMR

SEQ ID NO: 3 - protein, 20 aa, synthetic construct
IVGGHEAQPHSRPYMASLQM

SEQ ID NO: 4 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 5 - protein, 20 aa, synthetic construct
PRWDGERLYQEARKIVGAMV

SEQ ID NO: 6 - protein, 27 aa, Homo sapiens
MAHRPPSPALASVLLALLLSGAARAAE

SEQ ID NO: 7 - DNA, 903 bp, Homo sapiens
  source 1..903: mol_type = other DNA
gattggctataagaggagcttgatcgtgggtgcaccctggaccccaccatggctcaccggccccccagccctgccctggcgtccgtgctgctggccttgctgctgagcggtgctgcccgagctgcggagatcgtgggcgggcacgaggcgcagccacactcccggccctacatggcctccctgcagatgcgggggaacccgggcagccacttctgcggaggcaccttgatccaccccagcttcgtgctgacggccccgcactgcctgcgggacataccccagcgcctggtgaacgtggtgctcggagcccacaacgtgcggacgcaggagcccacccagcagcacttctcggtggctcaggtgtttctgaacaactacgacgcggagaacaaactgaacgacattctcctcatccagctgagcagcccagccaacctcagtgcgtccgtcacctcagtccagctgccacagcaggaccagccagtgccccacggcacccagtgcctggccatgggctggggccgcgtgggtgcccacgaccccccagcccaggtcctgcaggagctcaatgtcaccgtggtgaccttcttctgccggccacataacatttgcactttcgtccctcgccgcaaggccggcatctgcttcggagactcaggtggccccctgatctgtgatggcatcatccaaggaatagactccttcgtgatctggggatgtgccacccgccttttccctgacttcttcacgcgggtagccctctacgtggactggatccgttctacgctgcgccgtgtggaggccaagggccgcccctgaaccgcccctcccacagcgctggccgggaccccgagcctggctccaaaccctcgaggcggatctttggacagaagcagctcttgt

SEQ ID NO: 8 - protein, 48 aa, Homo sapiens
MGVPFFSSLRCMVDLGPCWAGGLTAEMKLLLALAGLLAILATPQPSEG

SEQ ID NO: 9 - DNA, 2568 bp, Homo sapiens
  source 1..2568: mol_type = other DNA
gtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtg

SEQ ID NO: 10 - protein, 11 aa, synthetic construct
RLYQEARKIVG

SEQ ID NO: 11 - protein, 11 aa, synthetic construct
GHEAQPHSRPY

SEQ ID NO: 12 - protein, 7 aa, synthetic construct
TQEPTQQ

SEQ ID NO: 13 - protein, 17 aa, synthetic construct
ATVQLPQQDQPVPHGTQ

SEQ ID NO: 14 - protein, 8 aa, synthetic construct
RVGAHDPP

SEQ ID NO: 15 - protein, 7 aa, synthetic construct
FCRPHNI

SEQ ID NO: 16 - protein, 15 aa, synthetic construct
PRRKAGICFGDSGGP

SEQ ID NO: 17 - protein, 7 aa, synthetic construct
IDSFVIW

SEQ ID NO: 18 - protein, 6 aa, synthetic construct
PYMASL

SEQ ID NO: 19 - protein, 14 aa, synthetic construct
AHCLRDIPQRLVNV

SEQ ID NO: 20 - protein, 16 aa, synthetic construct
HGTQCLAMGWGRVGAH

SEQ ID NO: 21 - protein, 14 aa, synthetic construct
NICTFVPRRKAGIC

SEQ ID NO: 22 - protein, 15 aa, synthetic construct
CATRLFPDFFTRVAL

SEQ ID NO: 23 - protein, 15 aa, synthetic construct
WIRSTRLLVEAKGRP

SEQ ID NO: 24 - protein, 196 aa, synthetic construct
VNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRK

SEQ ID NO: 25 - protein, 148 aa, synthetic construct
GYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 26 - protein, 13 aa, synthetic construct
RKIVGAMVQIITY

SEQ ID NO: 27 - protein, 15 aa, synthetic construct
RKIVGAMVQIITYRD

SEQ ID NO: 28 - protein, 10 aa, synthetic construct
WTPGVKRNGF

SEQ ID NO: 29 - protein, 12 aa, synthetic construct
RLDNRYQPMEPN

SEQ ID NO: 30 - protein, 10 aa, synthetic construct
SARIPCFLAG

SEQ ID NO: 31 - protein, 10 aa, synthetic construct
WDGERLYQEA

SEQ ID NO: 32 - protein, 10 aa, synthetic construct
YRSYNDSVDP

SEQ ID NO: 33 - protein, 9 aa, synthetic construct
FMSNSYPRD

SEQ ID NO: 34 - protein, 10 aa, synthetic construct
GSASPMELLS

SEQ ID NO: 35 - protein, 25 aa, synthetic construct
MIASSVRHAVILLLVAVAMMAAVIA

SEQ ID NO: 36 - protein, 26 aa, unidentified
  source 1..26: note = Leishmania
MIASSVRHAVILLLVAVAMMAAAVIA

SEQ ID NO: 37 - protein, 15 aa, synthetic construct
GGGGANSTAPAPAPA

SEQ ID NO: 38 - protein, 20 aa, unidentified
  source 1..20: note = Leishmania
MASRLVRVLAAAMLVAAAVS

SEQ ID NO: 39 - protein, 25 aa, synthetic construct
MAHRPPSPALASVLLALLLSGAARA

SEQ ID NO: 40 - protein, 254 aa, synthetic construct
MAHRPPSPALASVLLALLLSGAARAIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTAACRPHNICTFVPRRKAGICFGDAGGPLICDGIIQGIDSFVIWGCATRAAPDFFTRVALYVDWIRSTLRRVEAKGRP

SEQ ID NO: 41 - protein, 279 aa, synthetic construct
MAHRPPSPALASVLLALLLSGAARAIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTAACRPHNICTFVPRRKAGICFGDAGGPLICDGIIQGIDSFVIWGCATRAAPDFFTRVALYVDWIRSTLRRVEAKGRPGGGGANSTAPAPAPAHHHHHHHHHH

SEQ ID NO: 42 - protein, 717 aa, synthetic construct
MASRLVRVLAAAMLVAAAVSAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDADLDFTPEPAARASFVTGVNCETSCVQQPPCFPLKIPPNDPRIKNQADCIPFFRSAPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 43 - protein, 727 aa, synthetic construct
MASRLVRVLAAAMLVAAAVSAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDADLDFTPEPAARASFVTGVNCETSCVQQPPCFPLKIPPNDPRIKNQADCIPFFRSAPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREASHHHHHHHHHH

SEQ ID NO: 44 - protein, 10 aa, synthetic construct
HHHHHHHHHH

SEQ ID NO: 45 - protein, 229 aa, synthetic construct
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 46 - protein, 697 aa, synthetic construct
AAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDADLDFTPEPAARASFVTGVNCETSCVQQPPCFPLKIPPNDPRIKNQADCIPFFRSAPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 47 - protein, 5 aa, synthetic construct
EAAAK

SEQ ID NO: 48 - protein, 5 aa, synthetic construct
  REGION >5: note = The entire sequence of amino acids 1-5 can be repeated one to twenty times.
GGGGS

(19) United States
(12) Patent Application Publication
(10) Pub. No.: US 2026/0055211 A1
(43) Pub. Date: Feb. 26, 2026

(54) GLYCOENGINEERED POLYPEPTIDES TARGETING ANTI-NEUTROPHIL AUTOANTIBODIES AND USES THEREOF

(71) Applicant: GlycoEra AG, Wädenswil (CH)

(72) Inventors: Ganesh Venkataraman Kaundinya, Bedford, MA (US); Tanmoy Chinmoy Ganguly, Westford, MA (US); Rainer Follador, Chur (CH); Manuela Mally, Watt (CH); Jonathan Albert Back, Villers-Le-Lac (FR); Michela Manni, Zürich (CH); Dominique Nicolas Sirena, Rüti (CH)

(21) Appl. No.: 19/144,978

(22) PCT Filed: Jan. 5, 2024
(86) PCT No.: PCT/IB24/50112

§ 371 Date: Jul. 1, 2025

Related U.S. Application Data
(60) Provisional application No. 63/478,558, filed on Jan. 5, 2023.

(51) Int. Cl. C07K 16/40; A61K 38/00; C12N 9/08; C12N 9/64

(52) U.S. Cl. CPC C07K 16/40; C12N 9/0065; C12N 9/6424; C12Y 111/02002; C12Y 304/21076; A61K 38/00; C07K 2317/77; C07K 2319/00

(57) ABSTRACT

Provided herein are glycoengineered polypeptides comprising a first moiety comprising one or more peptides that specifically binds to an anti-neutrophil autoantibody and a second moiety comprising one or more glycans conjugated to the first moiety at one or more glycosylation sites. Also provided herein are nucleic acid sequence encoding provided glycoengineered polypeptides. Further provided herein are compositions comprising glycoengineered polypeptides and/or nucleic acids encoding the same, as well as methods of making and using the same.

### CROSS REFERENCE TO RELATED APPLICATIONS

This application claims priority to and the benefit of U.S. Provisional Patent Application No. 63/478,558 filed on Jan. 5, 2023, the entire contents of which are hereby incorporated by reference in its entirety.

### BACKGROUND

Anti-neutrophil autoantibodies are commonly found in autoimmune diseases such as Anti-neutrophil cytoplasmic antibody (ANCA)-associated vasculitis (AAV) and are often implicated in disease pathogenesis.

### SUMMARY

The present disclosure identifies certain challenges with existing therapies used to treat diseases associated with anti-neutrophil antibodies (e.g., ANCA vasculitis). For example, the present disclosure identifies limitations of some of the most widely used treatments options. B-cell targeting therapies are one of the most common therapies for treating diseases associated with anti-neutrophil antibodies (e.g., ANCA vasculitis or AAV). However, these therapeutic options do not directly act on anti-neutrophil autoantibodies in circulation, tissue or organs; and do not reduce and/or deplete anti-neutrophil autoantibodies. Therefore, while the currently available treatment options may provide some relief to patients, these treatment options do not treat the underlying cause of disease.

The present disclosure also identifies limitations in certain non-specific B-cell directed therapies (such as Rituximab) which are used for treating ANCA vasculitis or AAV. Such non-specific depletion of B cells is not desirable and can lead to unwanted side effects such as organ damage or immunosuppression. Additionally, non-specific B-cell directed therapies likely need to be dosed for a longer period of time to achieve a desired result, e.g., a clinical response. Furthermore, non-specific B-cell directed therapies likely cannot produce rapid responses due to the lack of specificity and lack of targeting of disease-causing components, e.g., anti-neutrophil autoantibodies or immune complexes comprising the same. Such non-specific therapies are also not able to disrupt and/or remove anti-neutrophil autoantibodies or pathogenic immune complexes comprising the same.

Without wishing to be bound by any particular theory, the technologies provided in the present disclosure can address certain limitations identified in existing therapies used to treat diseases associated with anti-neutrophil antibodies (e.g., ANCA vasculitis or AAV). Technologies provided herein specifically target autoantibodies or immune complexes comprising the same, and produce rapid, specific, and durable responses when administered to a subject. In some embodiments, technologies provided herein are anticipated to produce fewer and/or less severe unwanted side effects than non-specific B-cell directed therapies, due to the specificity in targeting immunogenic autoantibodies. In some embodiments, technologies provided herein can result in responses (e.g., clinical responses) in a shorter amount of time, as compared to non-specific B-cell directed therapies. In some embodiments, technologies provided herein do not need to be dosed for as long an extended period of time, e.g., as compared to non-specific B-cell directed therapies. In some embodiments, technologies provided herein can disrupt and/or remove immune complexes comprising autoantibodies disclosed herein. In some embodiments, technologies provided herein can result in improved depletion and/or removal of anti-neutrophil autoantibodies or immune complexes comprising the same, as compared to non-specific B-cell directed therapies. Advantages associated with technologies disclosed herein can result in improved responses (e.g., clinical responses) in patients having or at risk of having diseases associated with anti-neutrophil antibodies (e.g., ANCA vasculitis or AAV).

Among other things, the present disclosure provides technologies for reducing and/or removing anti-neutrophil autoantibodies by providing glycoengineered polypeptides that can simultaneously bind to a target autoantibody (e.g., an anti-neutrophil autoantibody) and to an endocytic receptor via one or more glycans. Without wishing to be bound by theory, the present disclosure proposes that binding of a glycoengineered polypeptide disclosed herein to a target autoantibody and to an endocytic receptor induces internalization of the target autoantibody into a cell. In some embodiments, internalization of the target autoantibody results in degradation. The technologies disclosed herein also relate to nucleic acid molecules encoding glycoengineered polypeptides disclosed herein. Further provided herein are compositions comprising glycoengineered polypeptides disclosed herein or nucleic acid molecules encoding the same, and methods of making the same.

Technologies provided herein are particularly useful for reducing and/or removing anti-neutrophil autoantibodies in subjects having, or at risk of having diseases associated with anti-neutrophil antibodies (e.g., ANCA vasculitis). Administration of compositions comprising glycoengineered polypeptides disclosed herein or nucleic acid molecules encoding the same can reduce and/or deplete anti-neutrophil antibodies, thus treating the disease or ameliorating one or more symptoms of the disease.

Accordingly, the present disclosure provides a glycoengineered polypeptide comprising: (a) a first moiety comprising one or more peptides that specifically binds to an anti-neutrophil autoantibody or a fragment or a complex thereof; and (b) a second moiety comprising one or more glycans conjugated to the first moiety at one or more glycosylation sites.

In some embodiments, an anti-neutrophil autoantibody is an anti-Proteinase 3 (PR3) autoantibody, or a fragment or a complex thereof. In some embodiments, an anti-PR3 autoantibody binds to PR3, or a variant or fragment thereof. In some embodiments, an anti-PR3 autoantibody binds to PR3 in complex with one or more proteins. In some embodiments, one or more proteins in complex with PR3 comprises CD177.

In some embodiments, an anti-neutrophil autoantibody is an anti-Myeloperoxidase (MPO) autoantibody or a fragment or a complex thereof. In some embodiments, an anti-MPO autoantibody binds to MPO, or a variant or fragment thereof.

In some embodiments, a glycoengineered polypeptide is capable of binding to: (a) an anti-PR3 autoantibody or a fragment or a complex thereof, and (b) an anti-MPO autoantibody or a fragment or a complex thereof.

In some embodiments, a second moiety specifically binds to one or more endocytic receptors.

In some embodiments, an endocytic receptor is or comprises an endocytic lectin. In some embodiments, an endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

In some embodiments, an endocytic receptor is ASGPR or a fragment or variant thereof, or a complex comprising ASGPR.

In some embodiments, a glycan structure comprises a biantennary structure. In some embodiments, a glycan structure comprises a biantennary GalNAc. In some embodiments, a biantennary GalNac binds to an asialoglycoprotein receptor (ASGPR) or a fragment or variant thereof, or a complex comprising ASGPR.

This disclosure also provides a composition comprising a glycoengineered polypeptide disclosed herein or a composition comprising a polynucleotide encoding a glycoengineered polypeptide disclosed herein.

In some embodiments, a composition comprises a glycoengineered polypeptide comprising a first moiety that specifically binds to an anti-PR3 autoantibody or a fragment or a complex thereof.

In some embodiments, a composition comprises a glycoengineered polypeptide comprising a first moiety that specifically binds to an anti-MPO autoantibody or a fragment or a complex thereof.

In some embodiments, a composition comprises: (i) a first glycoengineered polypeptide comprising a first moiety that specifically binds to an anti-PR3 autoantibody or a fragment or a complex thereof, and (ii) a second glycoengineered polypeptide comprising a first moiety that specifically binds to an anti-MPO autoantibody or a fragment or a complex thereof. In some embodiments, a ratio of the first glycoengineered polypeptide to the second glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

Also provided herein is a Leishmania host cell expressing a glycoengineered polypeptide disclosed herein.

The present disclosure further provides a method comprising: administering to a subject a pharmaceutical composition comprising: a glycoengineered polypeptide disclosed herein or a polynucleotide encoding a glycoengineered polypeptide disclosed herein.

In some embodiments, a subject has or is diagnosed as having anti-neutrophil cytoplasmic antibody (ANCA) vasculitis.

In some embodiments, ANCA vasculitis is Granulomatosis with Polyangiitis (GPA)/cytoplasmic ANCA (Formerly called Wegener's Granulomatosis).

In some embodiments, ANCA vasculitis is Microscopic Polyangiitis (MPA)/perinuclear ANCA.

In some embodiments, a glycoengineered polypeptide is capable of simultaneously binding to the target with the first moiety and binding to an endocytic receptor-expressing cell with the second moiety, thereby causing the target to be internalized into the cell. In some embodiments, internalization comprises transporting to a lysosome and/or degradation.

In some embodiments, administration of the pharmaceutical composition reduces a level of anti-neutrophil antibody as compared to a subject who has not been administered the pharmaceutical composition or as compared to the same subject prior to administration of the pharmaceutical composition. In some embodiments, a reduction in the level of the anti-neutrophil autoantibody prevents neutrophil activation.

In some embodiments, administration of the pharmaceutical compositions alleviates one or more symptoms of ANCA-vasculitis.

Further provided herein is a method comprising, assessing a level of an anti-neutrophil autoantibody in a sample from a subject, and administering a pharmaceutical composition comprising: a glycoengineered polypeptide disclosed herein or a polynucleotide encoding a glycoengineered polypeptide disclosed herein, if the level of the anti-neutrophil autoantibody is higher than a comparator.

In some embodiments, a comparator comprises a predetermined reference sample such as a sample obtained from an otherwise similar subject who does not have a disease or disorder, or a symptom of a disease or disorder.

In some embodiments, an anti-neutrophil autoantibody comprises: an anti-PR3 autoantibody or a fragment or a complex thereof; or an anti-MPO autoantibody or a fragment or a complex thereof, or a combination thereof.

In some embodiments, a subject has or is at risk of having ANCA vasculitis.

In some embodiments, a method is a treatment method.

In some embodiments, a method is a prevention method.

Additional features of glycoengineered polypeptides disclosed herein, nucleic acids encoding the same, composition comprising glycoengineered polypeptides or nucleic acids encoding the same, and methods of making and using the same are provided throughout the present disclosure.

### BRIEF DESCRIPTION OF DRAWINGS

FIGS. 1A-1B depict the structure of PR3 in its described tetrameric form (FIG. 1A) and as a monomer with N-glycosites (N129; N174), a Catalytic Triad (H71, D118, S203), and membrane binding residues (F180, F181, L228, F229), indicated by arrows (FIG. 1B). The source structure RCSB 1FUJ was used to visualize the described forms.

FIGS. 2A-2B show the binding kinetics of an exemplary PR3 glycoengineered polypeptide to recombinant anti-PR3 antibody CLB12.8 (FIG. 2A) and biotinylated recombinant human ASGPR1 (FIG. 2B). SPR binding data were collected in single cycle kinetic experiments using a Biacore 8K.

FIGS. 3A-E depict blocking potency of an exemplary PR3 glycoengineered polypeptide against PR3+ANCA patient autoantibodies from five patient samples, compared to blocking potency of human neutrophil-derived PR3 (HN PR3). Serum samples from PR3+ANCA patients were incubated with the indicated concentrations (x-axis) of the PR3 glycoengineered polypeptide or HN PR3. Autoantibodies not blocked by the PR3 glycoengineered polypeptide or HN PR3 were evaluated by ELISA. Results are expressed as the percentage of residual autoantibodies left in the sample compared to untreated samples. For each concentration tested, the data is shown in paired bars: the left filled bar indicates the residual binding percentage after blocking with HN PR3, and the right unfilled bar indicates the residual binding percentage after blocking with the PR3 glycoengineered polypeptide.

FIGS. 4A-4C depict autoantibody titer-independent blocking potency of an exemplary PR3 glycoengineered polypeptide against PR3+ANCA patient autoantibodies. Fifteen serum samples from PR3+ANCA patients with low autoantibodies titers (FIG. 4A), medium autoantibodies titers (FIG. 4B) and high autoantibodies titers (FIG. 4C) were incubated with the indicated concentrations of the PR3 glycoengineered polypeptide (x-axis). Autoantibodies not blocked by the PR3 glycoengineered polypeptide were then evaluated by ELISA. Graphs show the residual autoantibodies left in the sample expressed as a percentage compared to untreated samples. The dotted-line represent 50% blockade of autoantibodies by the PR3 glycoengineered polypeptide.

FIGS. 5A-5B depict depletion potency of an exemplary PR3 glycoengineered polypeptide for anti-PR3 autoantibodies from ANCA patients. Serum samples from five PR3+ANCA patients were incubated with magnetic beads coated with the PR3 glycoengineered polypeptide, and samples containing the magnetic beads were placed on a magnet. Flow-through was collected and analyzed for autoantibody levels by ELISA. FIG. 5A shows antibody level expressed as international units (IU) of anti-PR3 IgG/mL in the indicated serum sample after depletion with a control protein (Ctrl) or with the PR3 glycoengineered polypeptide coated beads. FIG. 5B shows data from FIG. 5A expressed as a numeric percentage of anti-PR3 autoantibody depletion obtained with the PR3 glycoengineered polypeptide.

FIGS. 6A-6C depict the ability of complexes comprising an exemplary PR3 glycoengineered polypeptide and an anti-PR3 antibody to be internalized and degraded in hepatocytes. FIG. 6A is a schematic representation of the experimental protocol. FIG. 6B is a blot depicting the PR3 glycoengineered polypeptide protein levels using an anti-His antibody. The blot was re-probed with B-actin as a loading control. FIG. 6C is a blot depicting CLB12.8 (anti-PR3 mouse antibody) protein levels using an anti-mouse IgG antibody. The blot was re-probed with B-actin as a loading control. HC: Heavy chain. LC: Light chain.

FIG. 7 depicts ASGPR-dependent internalization of complexes comprising an exemplary PR3 glycoengineered polypeptide and an anti-PR3 antibody. Left side of the blot depicts data from HepG2 wild-type cells (WT). Right side of the blot depicts data from HepG2 ASGPR1 knockout cells (KO). The top panel of the entire blot shows CLB12.8 (anti-PR3 mouse antibody) protein levels using an anti-IgG antibody. The bottom panel shows β-actin levels as loading control. HC: Heavy chain. LC: Light chain.

FIG. 8 depicts blocking potency of an exemplary MPO glycoengineered polypeptide against MPO+ANCA patient autoantibodies. Serum samples from one MPO+ANCA patient were incubated with the indicated concentrations of the MPO glycoengineered polypeptide (x-axis). Autoantibodies not blocked by the MPO glycoengineered polypeptide were then evaluated by ELISA. The graph shows autoantibody level expressed as international units (IU) of anti-MPO IgG/mL. The dotted-line represents the ELISA lower limit of detection.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

MPO: The term “MPO” is used herein in reference to Myeloperoxidase polypeptides as understood in the art. MPO is a heme-containing peroxidase. MPO protein is encoded by the MPO gene. Amino acid sequences for full-length MPO and/or for nucleic acids that encode it can be found in a public database such as GenBank, UniProt and Swiss-Prot. For example, an amino acid sequence of human MPO (SEQ ID NO: 4, for which residues 1-45 represent the signal peptide, can be found as UniProt/Swiss-Prot Accession No. P05164; a nucleic acid sequence (SEQ ID NO: 9) encoding human MPO can be found GenBank: J02694.1. Those skilled in the art will appreciate that sequences presented in SEQ ID NOs: 4 and 9 are exemplary, and certain variations (including, for example, conservative substitutions in SEQ ID NO:4, codon-optimized variants of SEQ ID NO:9, etc) are understood to also be or encode human MPO; additionally, those skilled in the art will appreciate that homologs and orthologs of human MPO are known and/or knowable through the exercise of ordinary skill and which may be useful in the present invention, for example, based on degree of sequence identity, presence of one or more characteristic sequence elements, and/or one or more shared activities.

PR3: The term “PR3” is used herein in reference to Proteinase 3 (PR3) polypeptides as understood in the art. PR3 is a serine protease and is also known as Myeloblastin, AGP7, neutrophil proteinase 4, P29, or Wegener autoantigen. PR3 protein is encoded by the myeloblastin gene. Amino acid sequences for full-length PR3 and/or for nucleic acids that encode it can be found in a public database such as GenBank, UniProt and Swiss-Prot. For example, an amino acid sequence of human PR3 (SEQ ID NO: 1, for which residues 1-25 represent the signal peptide, can be found as UniProt/Swiss-Prot Accession No. P24158; a nucleic acid sequence (SEQ ID NO: 7) encoding human PR3 is associated with GenBank: M75154.1. Those skilled in the art will appreciate that sequences presented in SEQ ID NOs: 1 and 7 are exemplary, and certain variations (including, for example, conservative substitutions in SEQ ID NO:1, codon-optimized variants of SEQ ID NO:7, etc) are understood to also be or encode human PR3; additionally, those skilled in the art will appreciate that homologs and orthologs of human PR3 are known and/or knowable through the exercise of ordinary skill and which may be useful in the present invention, for example, based on degree of sequence identity, presence of one or more characteristic sequence elements, and/or one or more shared activities.

Glycans: As used herein, the term “glycan” refers to one or more saccharides or sugar chains that can be attached to a protein or lipid to form a glycoconjugate. A glycan conjugated to a protein forms a glycoprotein. A glycan conjugated to a nitrogen atom of an amino acid residue is an N-linked glycan and a glycan conjugated to an oxygen atom of an amino acid residue is an O-linked glycan. As will be appreciated by those of ordinary skill in the art, the structure of a glycan indicates if a specific glycan is an N-linked glycan.

Glycoengineered: As used herein, the term “glycoengineered,” or an equivalent thereof means a process of glycosylating a target protein (e.g., a glycoengineered polypeptide disclosed herein), or a target protein made by such process. In some embodiments, the process uses a host cell system that has one or more enzymes (e.g., pathways) that provides for glycosylation of the target protein; in some other embodiments, the process is performed by chemically attaching one or more glycans to a target protein, e.g., using Click chemistry. Such a host cell system can be genetically engineered to introduce a glycosylation pathway to selectively glycosylate a target protein with a particular glycan structure. A host cell used to generate a glycoengineered target protein can include, for example, a recombinant nucleic acid encoding a target protein; and a recombinant nucleic acid encoding a heterologous glycosyltransferase. The host cell system used for glycoengineering (e.g., to generate a glycoengineered protein) can introduce, eliminate or modify N-linked glycosylation. The host cell system used for glycoengineering (e.g, to generate a glycoengineered protein) can introduce, eliminate or modify O-linked glycosylation. The host cell used for glycoengineering or to generate a glycoengineered target protein can be a mammalian cell, an insect cell, a yeast cell, a bacterial cell, a plant cell, a microalgae, or a protozoa. The protozoa used for glycoengineering can be a species of Leishmania. A glycoengineered target protein also includes a target protein that has been engineered to be selectively glycosylated at one or more specific sites when generated in the host cell system.

Glycoengineered polypeptide: As used herein, a “glycoengineered polypeptide” is a polypeptide that mediates the internalization and/or degradation of a target protein by specifically binding to a target protein (e.g., an anti-neutrophil autoantibody) and engaging with one or more endocytic receptors. In some embodiments, binding (e.g., simultaneous binding) of a glycoengineered polypeptide to a target protein and an endocytic receptor internalizes a target protein and/or activates one or more degradation pathways.

Glycosylation site: As used herein, the term “glycosylation site” refers to a site of glycosylation in a protein. Such a glycosylation site, also referred to as a glycosite herein, can be naturally present in the amino acid sequence of a protein or recombinantly engineered into the protein by addition or substitution or deletion of amino acids. In some embodiments, a glycosylation site is present in a so-called glycotag that is fused to a glycoengineered polypeptide disclosed herein. In certain embodiments, a glycotag is fused to a protein to create a bispecific binding protein. As used herein a glycotag refers to a peptide containing consensus N-glycosylation site sequence fused to N- or a C-terminal or both termini of a protein or polypeptide. In some embodiments, the glycotag is fused to the C-terminus of the of the glycoengineered polypeptide disclosed herein via a peptide linker. In some embodiments, the glycotag is fused to the N-terminus of the glycoengineered polypeptide disclosed herein via a peptide linker. In some embodiments, the peptide linker is a consensus peptide sequence. In some embodiments, the consensus peptide sequence is 1, 2, 3, 4, 5, 6, 7 or more amino acid residues in length. In some embodiments, the bifunctional protein provided herein contains 1, 2, 3, 4, 5, 6, 7, 8, 9, 10 or more glycotags.

Endocytic receptor: As used herein, the term “endocytic receptor” refers to a receptor or a fragment thereof that binds to a target and internalizes the target into a cell. In some embodiments, an endocytic receptor recognizes and binds to one or more glycans on a target. In some embodiments, binding of an endocytic receptor to a target internalizes the target into a cell, e.g., into a lysosome or phagosome. In some embodiments, an endocytic receptor is or comprises an endocytic lectin. In some embodiments, an endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

Anti-neutrophil autoantibody As used herein, the term “anti-neutrophil autoantibody” refers to an antibody produced in an organism (e.g., a mammal) which specifically binds to an epitope on an antigen endogenous to a neutrophil (e.g., a neutrophil autoantigen). A neutrophil autoantigen is expressed by a neutrophil (e.g., present inside a neutrophil and/or on the surface of a neutrophil) and is encoded by a nucleic acid sequence naturally occurring in a neutrophil genome. In some embodiments, a neutrophil autoantigen comprises a PR3 polypeptide or a variant or fragment thereof, a MPO polypeptide or a variant or fragment thereof, or both. In some embodiments, an anti-neutrophil autoantibody is or comprises a full antibody, an antigen binding fragment of an antibody, or a complex thereof. In some embodiments, an anti-neutrophil autoantibody is or comprises an anti-PR3 autoantibody, or a fragment or complex thereof. In some embodiments, an anti-neutrophil autoantibody is or comprises an anti-MPO autoantibody, or a fragment or complex thereof

Disease associated with anti-neutrophil autoantibodies: As used herein, a “disease associated with an anti-neutrophil autoantibody” includes a disease, disorder, or condition in which one or more anti-neutrophil autoantibodies is present and/or can be detected. In some embodiments, a disease associated with an anti-neutrophil autoantibody comprises anti-neutrophil cytoplasmic antibody (ANCA) vasculitis, also referred to as ANCA-associated vasculitis (or AAV). In some embodiments, ANCA vasculitis comprises Granulomatosis with Polyangiitis (GPA)/cytoplasmic ANCA (Formerly called Wegener's Granulomatosis). In some embodiments, ANCA vasculitis comprises Microscopic Polyangiitis (MPA)/perinuclear ANCA. In some embodiments, ANCA vasculitis comprises ANCA vasculitis comprises renal-limited vasculitis. In some embodiments, ANCA vasculitis comprises eosinophilic granulomatous polyangitis (EGPA).

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system, for example to achieve delivery of an agent that is, or is included in or otherwise delivered by, the composition. Those of ordinary skill in the art will be aware of a variety of routes that may, in appropriate circumstances, be utilized for administration to a subject, for example an animal or a human. In some embodiments, an animal is a domestic animal, such as a companion animal, e.g., a dog or a cat; in some embodiments, an animal is an animal used in agriculture (e.g., farming [e.g., a cow, a sheep or a horse]) or for recreation. For example, in some embodiments, administration may be systemic or local. Those skilled in the art will be aware of appropriate administration routes for use with particular therapies described herein, for example which include bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be by injection (e.g., intramuscular, intravenous, or subcutaneous injection). In some embodiments, injection may involve bolus injection, drip, perfusion, or infusion. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, an antibody agent can be formulated for oral delivery. For example, one with skill in the art will understand that an antibody agent disclosed herein can be formulated for oral delivery using technologies developed by Oramed (https://www.oramed.com/) or Premas (https://www.premasbiotech.com/).

Adult: As used herein, the term “adult” refers to a human eighteen years of age or older. In some embodiments, a human adult has a weight within the range of about 90 pounds to about 250 pounds.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments of a glycoengineered polypeptide disclosed herein, a first moiety comprising one or more peptides that specifically bind to a target autoantibody (e.g., an anti-neutrophil autoantibody), has a high affinity to an autoantigen (e.g., a neutrophil autoantigen). In some embodiments, a high affinity is an affinity of about 100-1000 pM. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference “]. In some embodiments, affinity may be assessed relative to a contemporaneous reference; in some embodiments, affinity may be assessed relative to a historical reference. Typically, when affinity is assessed relative to a reference, it is assessed under comparable conditions.

Avidity: As is known in the art, “avidity” is a measure of the accumulated strength of multiple non-covalent interactions between two or more binding partners in a complex. Those skilled in the art are aware of a variety of assays that can be used to assess avidity, and will furthermore be aware of appropriate controls for such assays. In some embodiments, avidity can be determined by (1) a binding affinity of two or more binding partners in a complex; (2) valency of each of the binding partners in a complex; and/or (3) structural arrangements of two or more binding partners in a complex. In some embodiments, the avidity of binding between two or more binding partners is more than a sum of each binding affinity between the two or more binding partners. In some embodiments, avidity is also referred to as apparent affinity or functional affinity. In some embodiments of a glycoengineered polypeptide disclosed herein, a second moiety comprising one or more glycans which can bind to a receptor (e.g., an endocytic receptor) contributes to binding avidity of the glycoengineered polypeptide. In some embodiments, an endocytic receptor is ASGPR or a fragment or variant thereof. In some embodiments, avidity is assessed in a quantitative assay. In some embodiments, avidity is assessed over a plurality of concentrations. In some embodiments, avidity is assessed in the presence of one or more potential competitor entities (e.g., that might be present in a relevant—e.g., physiological-setting). In some embodiments, avidity may be assessed relative to a contemporaneous reference; in some embodiments, avidity may be assessed relative to a historical reference. Typically, when avidity is assessed relative to a reference, it is assessed under comparable conditions.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: as used herein refers to a member of the animal kingdom. In some embodiments, “animal” refers to humans; unless otherwise specified, in many embodiments, a human may be of either gender and/or at any stage of development. In some embodiments, “animal” refers to non-human animals; unless otherwise specified, in many embodiments, a non-human animal may be of any gender and/or at any stage of development. In certain embodiments, a non-human animal is a mammal (e.g., a rodent, a mouse, a rat, a rabbit, a monkey, a dog, a cat, a sheep, cattle, a primate, and/or a pig). In some embodiments, an animal may be, for example, a mammal, a bird, a reptile, an amphibian, a fish, an insect, a worm, etc., In some embodiments, an animal may be a transgenic animal, genetically engineered animal, and/or a clone.

Antibody: As used herein, the term “antibody” refers to a polypeptide that includes canonical immunoglobulin sequence elements sufficient to confer specific binding to a particular target antigen. In the case of an autoimmune disease, the antigen to which a pathogenic autoantibody binds is also referred to as an “autoantigen.” As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. In some embodiments, antibodies produced and/or utilized in accordance with the present disclosure include one or more modifications on an Fc domain, e.g., an effector null mutation, e.g., a LALA, LAGA, FEGG, AAGG, or AAGA mutation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of dog, cat, mouse, rabbit, primate, or human antibodies. In some embodiments, antibody sequence elements are human, humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., VHH [e.g., a camelid VHH] or NAR) alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, trifunctional antibodies, diabodies, and minibodies, etc. In some embodiments, relevant formats may be or include: Adnectins®; Affibodies®; Affilins®; Anticalins®; Avimers®; BITE®s; cameloid antibodies; Centyrins®; ankyrin repeat proteins or DARPINs®; dual-affinity re-targeting (DART) agents; Fynomers®; shark single domain antibodies such as IgNAR; immune mobilixing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody format is or comprises a VHH, e.g., a camelid VHH. In some embodiments, a VHH is a multivalent VHH, e.g., a bivalent VHH. In some embodiments, an antibody comprises a single domain antibody, e.g., comprising one or more additional domains such as an Fc, a half-Fc (e.g., comprising an interchain cysteine mutant), an albumin domain, or combinations thereof. In some embodiments, an antibody comprises a single chain Fv, e.g., comprising one or more additional domains such as an Fc, a half-Fc (e.g., comprising an interchain cysteine mutant), an albumin domain, or combinations thereof. In some embodiments, an antibody comprises a polypeptide-Fc fusion. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (i.e., by expression of an engineered nucleic acid sequence. In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed subject matter.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Conjugate: The term “conjugate” as used herein refers to linking of one moiety to another moiety byzz in vitro methods (e.g., chemical synthesis) or in vivo (e.g., in a cell). In some embodiments, a moiety comprising one or more glycans (e.g., a second moiety) is conjugated to a different moiety, for example, at one or more glycosylation sites in vivo in a cell. In some embodiments, a moiety comprising one or more glycans (e.g., a second moiety) is conjugated to a different moiety, for example, at one or more glycosylation sites by chemical conjugation.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, alpha-helix character, alpha-sheet character, coiled-coil character, random coil character, etc.), and/or by a particular functional feature (e.g., binding activity, enzymatic activity, folding activity, signaling activity, etc.).

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Human: In some embodiments, a human is an embryo, a fetus, an infant, a child, a teenager, an adult, or a senior citizen.

Humanized: as is known in the art, the term “humanized” is commonly used to refer to antibodies (or antibody components) whose amino acid sequence includes VH and VL region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized VL region. In some embodiments, a humanized antibody only contains a humanized VH region. In some certain embodiments, a humanized antibody contains humanized VH and VL regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc, which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having not more than a small number (e.g., 3, 2, or 1) an amino acid sequence changes relative to that of a reference CDR. In some embodiments, a CDR that is substantially identical to a reference CDR differs from that reference CDR by one or more amino acid changes at the end of the reference CDR; in some such embodiments, the relevant CDR is identical to the reference CDR other than at one or both ends. As is known in the art, CDR elements typically have a length within a range of a few amino acids (e.g., 3, 4, 5, 6, or 7) to about 20 or 30 amino acids (see, for example, Collis et al. J. Mol. Biol. 325:337, 2003, incorporated herein by reference); thus, in some embodiments, a CDR may be considered to be substantially identical to a reference CDR when it shares at least about 80% (or less for a shorter CDR), at least about 85%, at least about 90%, at least about 95%, at least about 98%, at least about 99%, or at least about 100% identity with the reference CDR.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in the pertinent art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues

Treat: As used herein, the term “treat,” “treatment,” or “treating” is used to refer to one or more of partial or complete alleviation, amelioration, relief, inhibition, prevention, delay of onset of, reduction in severity of and/or reduction in frequency (e.g., incidence) of one or more symptoms or features of a disease, disorder, and/or condition. In some embodiments, treatment may be prophylactic; for example may be administered to a subject who does not exhibit signs of a disease, disorder, and/or condition. In some embodiments, treatment may be administered to a subject who exhibits early signs of the disease, disorder, and/or condition, and may, for example, decrease risk of developing pathology associated with the disease, disorder, and/or condition and/or delay onset and/or decrease rate of development or worsening of one or more features of a disease, disorder and/or condition.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors, e.g., that are statistically correlated with increased risk of development of the relevant disease, disorder, and/or condition. Thus, in some embodiments, treatment may be prophylactic; in some embodiments, treatment may be therapeutic.

Variant: The term “variant”, as used herein, refers to a molecule or entity (e.g., that are or comprise a nucleic acid, protein, or small molecule) that shows significant structural identity with a reference molecule or entity but differs structurally from the reference molecule or entity, e.g., in the presence or absence or in the level of one or more chemical moieties as compared to the reference molecule or entity. In some embodiments, a variant also differs functionally from its reference molecule or entity. In many embodiments, whether a particular molecule or entity is properly considered to be a “variant” of a reference is based on its degree of structural identity with the reference molecule. As will be appreciated by those skilled in the art, a biological or chemical reference molecule in typically characterized by certain characteristic structural elements. A variant, by definition, is a distinct molecule or entity that shares one or more such characteristic structural elements but differs in at least one aspect from the reference molecule or entity. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

Disclosed herein are glycoengineered polypeptides and compositions comprising the same having the ability to degrade one or more anti-neutrophil autoantibodies by binding to anti-neutrophil autoantibodies with a first moiety and binding to an endocytic receptor with a second moiety comprising one or more glycans, thus targeting the anti-neutrophil antibody for degradation. As exemplified herein, a glycoengineered polypeptide is engineered by introduction of glycosylation sites on a glycoengineered polypeptide, resulting in an engineered glycosylation profile that mediates endocytic receptor degradation of the glycoengineered polypeptides and the target to which it binds.

By customizing the N-glycosylation, a glycoengineered polypeptide described herein: 1) has homogeneous glycosylation; 2) can degrade large targets such as immune complexes; 3) has a defined ligand-to-antibody ratio; 4) has defined glycosylation sites; 6) can activate more diverse and powerful degradation receptors; and/or 6) can engage in protein degradation in a highly optimized manner. A glycoengineered polypeptide may be employed as a novel therapeutic to treat autoimmune diseases, e.g., a disease associated with anti-neutrophil autoantibodies, e.g., ANCA vasculitis.

### Anti-Neutrophil Autoantibodies

Neutrophils are the most common type of white blood cells. An anti-neutrophil autoantibody is an antibody that specifically binds to an antigen endogenous to neutrophils (e.g., a neutrophil autoantigen). A neutrophil autoantigen is encoded by a nucleic acid sequence that is naturally occurring in the genome of neutrophils. In some embodiments, a neutrophil autoantigen is expressed in a neutrophil, e.g., in the cytoplasm, nucleus, peri-nucleus, or in a compartment in a cell. In some embodiments, a neutrophil autoantigen is expressed on the cell surface of neutrophils.

In some embodiments, an anti-neutrophil autoantibody is an IgG antibody. In some embodiments, an anti-neutrophil autoantibody is an IgA antibody. In some embodiments, an anti-neutrophil autoantibody is an IgM antibody. In some embodiments, an anti-neutrophil autoantibody is an IgD antibody. In some embodiments, an anti-neutrophil autoantibody is an IgE antibody.

Anti-neutrophil autoantibodies are produced and released from B cells. Previous reports have shown that antigens such as microbial components may stimulate the expression of neutrophil autoantigens, e.g., such as PR3 and/or MPO, on the surface of neutrophils (Fijolet, J. et al. Clinical Rheumatology, 2019). Upon binding of anti-neutrophil autoantibodies to neutrophil antigens, e.g., PR3 and/or MPO, the autoantibodies may activate neutrophils. Once activated, neutrophils may attach to the endothelium of the blood vessels and release reactive oxygen species, inflammatory cytokines, toxic substances, and neutrophil extracellular traps (NETs), leading to systemic vasculitis and injuries to multiple organs. Adhesion of neutrophils in target tissues, particularly the kidney, by means of the release of injurious oxidants and enzymes.

Exemplary neutrophil autoantigens include, but are not limited to, proteinase 3 (PR3) or a variant or fragment thereof; myeloperoxidase (MPO) or a variant or fragment thereof; lysosomal membrane protein-2 (LAMP2) or a variant or fragment thereof; pentraxin-3 (PTX3) or a variant or fragment thereof. In some embodiments, anti-neutrophil autoantibodies may specifically bind neutrophil autoantigens (e.g., cytoplasmic granule proteins), such as PR3, MPO, LAMP2 and/or PTX3. The peptide sequence of human PR3 is designated by UNIPROT protein number P24158. The peptide sequence of human MPO is designated by UNIPROT protein number P05164. The peptide sequence of human LAMP2 is designated by UNIPROT protein number P13473. The peptide sequence of human PTX3 is designated by UNIPROT protein number P26022. These peptide sequences, as well as other information available via the UNIPROT database are incorporated herein by reference. See www.uniprot.org.

In some cases, anti-neutrophil cytoplasmic antibodies have been associated with administration of various drugs, most notably, the antithyroid drug propylthiouracil (PTU). Gao and Zhao (2009) Nephrology 14(1): 33-41.

### Anti-Neutrophil Autoantibody Associated Diseases

Anti-neutrophil autoantibodies are detected in a number of autoimmune disorders. In some embodiments, anti-neutrophil autoantibodies may cause neutrophil activation leading to vascular endothelial injury to small and/or medium blood vessels. In some embodiments, anti-neutrophil autoantibodies contribute to and/or result in vasculitis. In some embodiments, anti-neutrophil autoantibodies contribute to and/or result in anti-neutrophil cytoplasmic antibody (ANCA) vasculitis.

In some embodiments, diseases associated with anti-neutrophil autoantibodies are associated with increased and/or aberrant expression of one or more neutrophil autoantigens. Without wishing to be bound by any particular theory, it is proposed that antigens, such as microbial components, may stimulate the expression of neutrophil autoantigens on the cell surface of neutrophils and/or induce aberrant transcription of neutrophil autoantigens, e.g., due to epigenetic deregulation (Fijolet, J. et al. Clinical Rheumatology, (2019); Jennette C. J. et al., Curr Opin Nephrol Hypertens (2011) 20(3): 263-270).

### ANCA Vasculitis

ANCA vasculitis is a systemic disease that may involve ears, nose and throat, lungs, kidneys, heart, digestive system, nervous system, eyes, skin, musculoskeletal tract and, infrequently, other organs (Hilhorst, M. et al. J Am Soc Neprhol, 2015). ANCA vasculitis may be induced by autoimmunity (e.g., anti-neutrophil autoantibodies) to neutrophil granule proteins (e.g., neutrophil autoantigens), such as myeloperoxidase (MPO) or proteinase 3 (PR3) as described herein. Anti-neutrophil autoantibodies against PR3 or MPO can alone or in combination result in vasculitis (e.g., small to medium-vessel vasculitis). In some embodiments, anti-neutrophil autoantibodies that specifically bind to PR3 and/or MPO cause ANCA vasculitis. Additional anti-neutrophil autoantibodies have been identified in subjects with ANCA vasculitis, which may be causative of or contribute to development or severity of a neutrophil autoantibody associated disease, including LAMP-2 and PTX3.

Current treatments for ANCA vasculitis include cyclophosphamide, RITUXIMAB, methotrexate, mycophenolate mofetil, steroids, and plasma exchange. In some embodiments, one or more additional treatments (e.g., one or more current treatments listed above) may be administered prior to, substantially simultaneously with, or subsequent to one or more provided glycoengineered polypeptides.

### Microscopic Polyangiitis (MPA)/Perinuclear ANCA

Autoimmunity to MPO (e.g., generation of anti-MPO autoantibodies) is strongly associated with Microscopic Polyangiitis (MPA)/perinuclear ANCA. MPA is often characterized by vasculitis limited to the kidneys. Studies have shown that 1 out of 3 patients with MPA progress to dialysis or kidney transplantation (Hilhorst, M. et al. J Am Soc Neprhol, 2015).

In some embodiments, ANCA vasculitis is Microscopic Polyangiitis (MPA)/perinuclear ANCA.

Current treatments for MPA vasculitis include immune suppression therapy using glucocorticoids (e.g., methyl prednisone), cyclophosphamide, RITUXIMAB, methotrexate, mycophenolate mofetil, azathioprine, steroids, and plasma exchange. In some embodiments, one or more additional treatments (e.g., one or more current treatments listed above) may be administered prior to, substantially simultaneously with, or subsequent to one or more provided glycoengineered polypeptides.

Granulomatosis with Polyangiitis (GPA)/Cytoplasmic ANCA

Autoimmunity to PR3 (e.g., generation of PR3 autoantibodies) is strongly associated with Granulomatosis with Polyangiitis (GPA)/cytoplasmic ANCA (Formerly called Wegener's Granulomatosis). GPA if often characterized by granulomatous inflammation of the respiratory tract, necrotizing small-vessel vasculitis, and glomerulonephritis. A hallmark of GPA is granulomatous inflammation. Granuloma formation is thought to be initiated by small aggregates of neutrophils surrounding necrotic areas (microabscess) (Hilhorst, M. et al. J Am Soc Neprhol, 2015).

In some embodiments, ANCA vasculitis is Granulomatosis with Polyangiitis (GPA)/cytoplasmic ANCA.

Available treatment options for patients that present with anti-neutrophil autoantibodies can include administration of immunosuppressants, such as methotrexate, cyclophosphamide, rituximab and/or steroids (e.g., corticosteroids). Rituximab might be effective in management of GPA and/or MPA, but has a delay in onset and carries high infection risk. Maintenance of remission therapies may be achieved with methotrexate and optionally CTL4-immunoglobulins. For some patients plasma exchange can be beneficial.

### Glycoengineered Polypeptides

Disclosed herein are glycoengineered polypeptides comprising: (a) a first moiety comprising one or more peptides that specifically binds to an anti-neutrophil autoantibody or a fragment or a complex thereof; and (b) a second moiety comprising one or more glycans conjugated to the first moiety at one or more glycosylation sites.

In some embodiments, an anti-neutrophil autoantibody is an anti-Proteinase 3 (PR3) autoantibody or a fragment or a complex thereof. In some embodiments, an anti-PR3 autoantibody binds to PR3, or a variant or fragment thereof. In some embodiments, an anti-PR3 autoantibody binds to PR3 in complex with one or more proteins. In some embodiments, the one or more proteins in complex with PR3 comprises CD177.

In some embodiments, an anti-neutrophil autoantibody is an anti-Myeloperoxidase (MPO) autoantibody or a fragment or a complex thereof. In some embodiments, an anti-MPO autoantibody binds to MPO, or a variant or fragment thereof.

In some embodiments, a glycoengineered polypeptide is capable of binding to: an anti-PR3 autoantibody or a fragment or a complex thereof, and an anti-MPO autoantibody or a fragment or a complex thereof.

In some embodiments, a glycoengineered polypeptide is capable of binding to one or more anti-neutrophil autoantibodies, in addition to an anti-PR3 autoantibody and/or an anti-MPO autoantibody.

In some embodiments, a glycoengineered polypeptide disclosed herein comprises a first moiety, a second moiety and one or more additional elements. In some embodiments, a glycoengineered polypeptide comprises: an N-glycosylation site, a linker, a spacer, a signal peptide, a tag, a half-life extender or a combination thereof.

In some embodiments, a glycoengineered polypeptide comprises one or more N-glycosylation sites in a first moiety. In some embodiments, a first moiety comprises one or more N-glycosylation sites that are naturally occurring and/or one or more N-glycosylation sites that are engineered into a first moiety. In some embodiments, an engineered N-glycosylation site (also referred to herein as a glycosite or a glycotag) is or comprises the sequence of GGGGANSTAPAPAPA (SEQ ID NO: 37).

In some embodiments, a glycoengineered polypeptide comprises a linker. In some embodiments, a linker comprises a Gly-Ser linker, or an EAAAK linker. In some embodiments, a linker comprises a (Gly-Gly-Gly-Gly-Ser) n linker, wherein n is an integer between 0 to 20.

In some embodiments, a glycoengineered polypeptide comprises a spacer. In some embodiments, a spacer comprises one or more nucleotides which separates a first nucleic acid sequence from a subsequent nucleic acid sequence.

In some embodiments, a spacer comprises a nucleic acid sequence which encodes one or more peptides that separates a first encoded polypeptide sequence from a subsequent encoded polypeptide sequence.

In some embodiments, a glycoengineered polypeptide comprises a signal peptide, e.g., as disclosed herein. In some embodiments, a signal peptide is a native signal peptide. In some embodiments, a signal peptide is not a native signal peptide.

In some embodiments, a signal peptide is derived from a Leishmania species. In certain embodiments, a signal peptide is derived from Leishmania tarentolae. In certain embodiments, a signal peptide is derived from Leishmania major.

In certain embodiments, the signal peptide is an invertase signal peptide derived from Leishmania tarentolae.

In certain embodiments, the signal peptide is an alkaline phosphatase signal peptide derived from Leishmania major.

In certain embodiments, a signal peptide comprises an amino acid sequence of SEQ ID NO: 35, or a portion thereof. In certain embodiments, a signal peptide comprises an amino acid sequence of SEQ ID NO: 36, or a portion thereof. In certain embodiments, a signal peptide comprises an amino acid sequence of SEQ ID NO: 38, or a portion thereof. In certain embodiments, a signal peptide is processed and removed from a glycoengineered polypeptide.

Exemplary signal peptide: SPinv, a modified signal peptide from Leishmania tarentolae invertase, SEQ ID NO: 35: MIASSVRHAVILLLVAVAMMAAVIA

Exemplary signal peptide: SPinv, the native signal peptide from Leishmania tarentolae invertase, SEQ ID NO: 36: MIASSVRHAVILLLVAVAMMAAAVIA.

Exemplary signal peptide: native signal peptide from Leishmania major alkaline phosphatase, SEQ ID NO: 38: MASRLVRVLAAAMLVAAAVS.

In some embodiments, a glycoengineered polypeptide comprises a tag. In some embodiments, a tag is a moiety that can be used for purifying and/or identifying a glycoengineered polypeptide disclosed herein. In some embodiments, a tag is not a glycotag. In some embodiments, a tag comprises a His tag, a Myc tag, or a GST tag, In some embodiments, a tag comprises a cleavable tag.

In some embodiments, a tag is a His tag (HHHHHHHHHH; SEQ ID NO: 44)

In some embodiments, a half-life extender comprises albumin or a fragment or a variant thereof.

In some embodiments, a half-life extender comprises a Fc domain, e.g., with or without mutations in an Fc domain.

### First Moiety

In some embodiments, a glycoengineered polypeptide comprises a first moiety comprising one or more peptides that specifically binds to an anti-neutrophil autoantibody or a fragment thereof.

In some embodiments, the one or more peptides comprises an epitope from a neutrophil antigen which is recognized by an anti-neutrophil autoantibody or a fragment thereof (e.g., an antigen binding fragment of an anti-neutrophil autoantibody). In some embodiments, an epitope comprises a fragment of a neutrophil antigen, e.g., an inactive fragment of a neutrophil antigen and/or a soluble fragment of a neutrophil antigen. In some embodiments, an epitope comprises an extracellular fragment of a neutrophil antigen.

In some embodiments, a first moiety comprising one or more peptides that specifically binds to an anti-neutrophil autoantibody or a fragment or a complex thereof comprises one or more idiotopes specific to an anti-neutrophil autoantibody or a fragment thereof. In some embodiments, a first moiety comprising one or more peptides that specifically binds to an anti-neutrophil autoantibody is an anti-idiotypic antibody or a fragment thereof.

In some embodiments, a first moiety comprises a peptide that is about 5 amino acids in length to about 500 amino acids in length. In some embodiments, a first moiety comprises a peptide that is about 5 amino acids, about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, about 100 amino acids, about 200 amino acids, about 300 amino acids, about 400 amino acids, about 500 amino acids in length.

In some embodiments, a first moiety comprises a peptide that is at least 5 amino acids, at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 200 amino acids, at least 300 amino acids, at least 400 amino acids, at least 500 amino acids in length.

In some embodiments, a first moiety comprises one or more peptides that are about 50 amino acids in length to about 5000 amino acids in length in total. In some embodiments, a first moiety comprises one or more peptides that are about 50 amino acids, about 60 amino acids, about 70 amino acids, about 80 amino acids, about 90 amino acids, about 100 amino acids, about 200 amino acids, about 300 amino acids, about 400 amino acids, about 500 amino acids, about 600 amino acids, about 700 amino acids, about 800 amino acids, about 900 amino acids, about 1000 amino acids, about 1500 amino acids, about 2000 amino acids, about 2500 amino acids, about 3000 amino acids, about 3500 amino acids, about 4000 amino acids, about 4500 amino acids, about 5000 amino acids in length in total.

In some embodiments, a first moiety comprises 1, 2, 3, 4, 5, or more peptides that specifically bind to an anti-neutrophil autoantibody.

In some embodiments, one or more peptides of a first moiety that specifically bind to an anti-neutrophil autoantibody are the same, e.g., the one or more peptides have the same sequence. In some embodiments, the one or more peptides having the same sequence are separated by one or more intervening sequences (e.g., spacers and/or linkers). In some embodiments, the one or more peptides having the same sequence are not separated by one or more intervening sequences.

In some embodiments, one or more peptides of a first moiety that specifically bind to an anti-neutrophil autoantibody are different, e.g., the one or more peptides do not have the same sequence. In some embodiments, the one or more peptides having different sequences are separated by one or more intervening sequences (e.g., spacers and/or linkers). In some embodiments, the one or more peptides different sequences are not separated by one or more intervening sequences (e.g., spacers and/or linkers).

In some embodiments, the one or more peptides having different sequences comprise one or more peptides that specifically bind to the same autoantibody. In some embodiments, the one or more peptides bind to different epitopes of an autoantibody (e.g., to different domains of the autoantibody). In some embodiments, the one or more peptides having different sequences comprise one or more peptides that specifically bind to an anti-PR3 autoantibody or a fragment thereof. In some embodiments, the one or more peptides having different sequences comprise one or more peptides that specifically bind to an anti-MPO autoantibody or a fragment thereof.

In some embodiments, the one or more peptides having different sequences comprise one or more peptides that specifically bind to a first autoantibody (e.g., an anti-PR3 autoantibody or a fragment thereof) and one or more peptides that bind to second autoantibody (e.g., an anti MPO autoantibody or a fragment thereof). In some embodiments, the one or more peptides having different sequences are separated by one or more intervening sequences (e.g., spacers and/or linkers). In some embodiments, the one or more peptides different sequences are not separated by one or more intervening sequences (e.g., spacers and/or linkers).

In some embodiments, a linker separating one or more peptides of a first moiety comprises a Gly-Ser linker, or an EAAAK linker. In some embodiments, a linker comprises a (Gly-Gly-Gly-Gly-Ser) n linker, wherein n is an integer between 0 to 20.

In some embodiments, a spacer separating one or more peptides of a first moiety comprises 1-10 amino acid residues, or about 10-20 amino acid residues.

In some embodiments, one or more peptides that specifically bind to an anti-neutrophil autoantibody are each conjugated to a second moiety.

In some embodiments, one or more peptides that specifically bind to an anti-neutrophil autoantibody are not each conjugated to the second moiety.

In some embodiments, one or more peptides that specifically bind to an anti-neutrophil autoantibody are conjugated to each other, e.g., are situated on one polypeptide.

In some embodiments, one or more peptides that specifically bind to an anti-neutrophil autoantibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an anti-neutrophil autoantibody are not separated by a protease cleavage site or an IRES, e.g., is expressed as a fusion protein.

In some embodiments, one or more peptides of a first moiety that specifically bind to an anti-neutrophil autoantibody comprise an epitope that is recognized by an anti-neutrophil autoantibody. In some embodiments, an epitope is a linear epitope. In some embodiments, an epitope is a conformational epitope.

In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by an anti-neutrophil autoantibody, and X denotes intervening amino acid sequences with n being an integer from 0-20. In some embodiments, an intervening amino acid sequence is a spacer or a linker, e.g., as described herein.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn . . . [An]-Xn, wherein A1 is a first portion of an epitope, A2 is a second portion of an epitope, and An is the n-th portion of an epitope which together form a spatial epitope that is recognized by an anti-neutrophil autoantibody, and X denotes intervening amino acid sequences with n being an integer from 0-20. In some embodiments, an intervening amino acid sequence is a spacer or a linker, e.g., as described herein.

In some embodiments, a first moiety comprises a plurality of epitopes, e.g., the same or different epitopes. In some embodiments, a first moiety comprises a plurality of the same epitopes, e.g., epitopes recognized by the same anti-neutrophil antibody. In some embodiments, a first moiety comprises a plurality of different epitopes, e.g., epitopes recognized by different anti-neutrophil antibodies. In some embodiments, the plurality of epitopes is separated by a linker, IRES or cleavage peptide.

In some embodiments, one or more peptides that specifically bind to an anti-neutrophil antibody comprises an antibody agent. In some embodiments, the antibody agent comprises an antigen binding fragment. In some embodiments, the antibody agent comprises a full antibody, a Fab fragment, an scFv, a nanobody, a duobody, a single domain antibody (e.g., a VHH). In some embodiments, the antibody agent comprises a VHH, e.g., a camelid VHH or a bivalent VHH.

### Proteinase 3 (PR3) and First Moiety Comprising PR3 Peptides

PR3 is a serine protease enzyme expressed mainly in neutrophils and monocytes. PR3 is typically present in azurophil granules of human polymorphonuclear leukocytes. PR-3 has broad proteolytic activity and degrades a variety of extracellular matrix proteins, including fibronectin, type IV collagen and laminin (Brockmann H et al., Arthritis Research & Therapy (2002) volume 4, Article number: 220). Typically, transcription of PR3 is turned off before neutrophils are released from the bone marrow, however in ANCA vasculitis increased and/or aberrant expression of PR3 has been reported. Additionally, ANCA vasculitis has also been shown to be associated with increased levels of PR3 on the surface of circulating neutrophils (Jennette C. J. et al., Curr Opin Nephrol Hypertens (2011) 20(3): 263-270).

A human PR3 polypeptide sequence is provided herein as SEQ ID NO: 1, with the bolded sequence denoting the signal peptide and the propeptide (AE, bold italicized) (corresponding to Uniprot Accession Number: P24158):

| MAHRPPSPALASVLLALLLSGAARAAEIVGGHEAQPHSRPYMASLQMRGN | |
|---|---|
| PGSHFCGGTLIHPSFVLTAAHCLRDIPQRLVNVVLGAHNVRTQEPTQQHFSVAQVFLNNYDAEN | |
| KLNDVLLIQLSSPANLSASVATVQLPQQDQPVPHGTQCLAMGWGRVGAHDPPAQVLQELNVT | |
| VVTFFCRPHNICTFVPRRKAGICFGDSGGPLICDGIIQGIDSFVIWGCATRLFPDFFTRVALYVDW | |
| IRSTLRRVEAKGRP | |
| Human PR3 signal peptide: | |
| (SEQ ID NO: 6) | |
| MAHRPPSPALASVLLALLLSGAARAAE. | |
| Human PR3 can be encoded by the following nucleic acid sequence from the | |
| myeoblastin gene | |
| (SEQ ID NO: 7) | |
| 1 | gattggctat aagaggagct tgatcgtggg tgcaccctgg accccaccat ggctcaccgg |
| 61 | ccccccagcc ctgccctggc gtccgtgctg ctggccttgc tgctgagcgg tgctgcccga |
| 121 | gctgcggaga tcgtgggcgg gcacgaggcg cagccacact cccggcccta catggcctcc |
| 181 | ctgcagatgc gggggaaccc gggcagccac ttctgcggag gcaccttgat ccaccccagc |
| 241 | ttcgtgctga cggccccgca ctgcctgcgg gacatacccc agcgcctggt gaacgtggtg |
| 301 | ctcggagccc acaacgtgcg gacgcaggag cccacccagc agcacttctc ggtggctcag |
| 361 | gtgtttctga acaactacga cgcggagaac aaactgaacg acattctcct catccagctg |
| 421 | agcagcccag ccaacctcag tgcgtccgtc acctcagtcc agctgccaca gcaggaccag |
| 481 | ccagtgcccc acggcaccca gtgcctggcc atgggctggg gccgcgtggg tgcccacgac |
| 541 | cccccagccc aggtcctgca ggagctcaat gtcaccgtgg tgaccttctt ctgccggcca |
| 601 | cataacattt gcactttcgt ccctcgccgc aaggccggca tctgcttcgg agactcaggt |
| 661 | ggccccctga tctgtgatgg catcatccaa ggaatagact ccttcgtgat ctggggatgt |
| 721 | gccacccgcc ttttccctga cttcttcacg cgggtagccc tctacgtgga ctggatccgt |
| 781 | tctacgctgc gccgtgtgga ggccaagggc cgcccctgaa ccgcccctcc cacagcgctg |
| 841 | gccgggaccc cgagcctggc tccaaaccct cgaggcggat ctttggacag aagcagctct |
| 901 | tgt |

In some embodiments, a PR3 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 1. In some embodiments, a PR3 polypeptide is or comprises SEQ ID NO: 1. In some embodiments, a PR3 polypeptide is or comprises SEQ ID NO: 1 without the signal peptide of SEQ ID NO: 6.

In some embodiments, a PR3 polypeptide comprises a sequence having at least 85% identity to SEQ ID NO: 1 without the signal peptide of SEQ ID NO: 6.

In some embodiments, a PR3 polypeptide comprising a sequence having at least 85% identity to SEQ ID NO: 1 without the signal peptide of SEQ ID NO: 6, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 1. In some embodiments, a first moiety comprises one or more peptides comprising the sequence of SEQ ID NO:1. In some embodiments, a first moiety comprises one or more peptides comprising the sequence of SEQ ID NO:1 without the signal peptide of SEQ ID NO: 6.

In some embodiments, a first moiety comprises one or more peptides comprising a sequence having at least 85% identity to SEQ ID NO: 1 without the signal peptide of SEQ ID NO: 6.

In some embodiments, a first moiety comprising one or more peptides comprising a sequence having at least 85% identity to SEQ ID NO: 1 without the signal peptide of SEQ ID NO: 6, further comprises a different signal peptide, e.g., as disclosed herein.

An exemplary PR3 polypeptide sequence is provided herein as SEQ ID NO: 40, with the bolded sequence denoting the truncated signal peptide:

MAHRPPSPALASVLLALLLSGAARAIVGGHEAQPHSRPYMASLQMR
GNPGSHFCGGTLIHPSFVLTAAHCLRDIPQRLVNVVLGAHNVRTQEPTQQHFSVAQVFL
NNYDAENKLNDVLLIQLSSPANLSASVATVQLPQQDQPVPHGTQCLAMGWGRVGAH
DPPAQVLQELNVTVVTAACRPHNICTFVPRRKAGICFGDAGGPLICDGIIQGIDSFVIWG
CATRAAPDFFTRVALYVDWIRSTLRRVEAKGRP
An engineered PR3 truncated signal peptide:
(SEQ ID NO: 39)
MAHRPPSPALASVLLALLLSGAARA

In some embodiments, a PR3 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 40. In some embodiments, a PR3 polypeptide is or comprises SEQ ID NO: 40. In some embodiments, a PR3 polypeptide is or comprises SEQ ID NO: 40 without the signal peptide of SEQ ID NO: 39.

In some embodiments, a PR3 polypeptide comprising a sequence having at least 85% identity to SEQ ID NO: 40 without the signal peptide of SEQ ID NO: 39, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 40. In some embodiments, a first moiety comprises one or more peptides comprising the sequence of SEQ ID NO: 40. In some embodiments, a first moiety comprises one or more peptides comprising the sequence of SEQ ID NO: 40 without the signal peptide of SEQ ID NO: 39.

In some embodiments, a first moiety comprises one or more peptides comprising a sequence having at least 85% identity to SEQ ID NO: 40 without the signal peptide of SEQ ID NO: 39.

In some embodiments, a first moiety comprising one or more peptides comprising a sequence having at least 85% identity to SEQ ID NO: 40 without the signal peptide of SEQ ID NO: 39, further comprises a different signal peptide, e.g., as disclosed herein.

An exemplary PR3 polypeptide sequence without a signal peptide is provided herein as SEQ ID NO: 45:

IVGGHEAQPHSRPYMASLQMRGNPGSHFCGGTLIHPSFVLTAAHCLRDIPQR
LVNVVLGAHNVRTQEPTQQHFSVAQVFLNNYDAENKLNDVLLIQLSSPANLSASVATVQLPQQ
DQPVPHGTQCLAMGWGRVGAHDPPAQVLQELNVTVVTFFCRPHNICTFVPRRKAGICFGDSGG
PLICDGIIQGIDSFVIWGCATRLFPDFFTRVALYVDWIRSTLRRVEAKGRP

In some embodiments, a PR3 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 45. In some embodiments, a PR3 polypeptide is or comprises SEQ ID NO: 45.

In some embodiments, a PR3 polypeptide comprising a sequence having at least 85% identity to SEQ ID NO: 45 further comprises a signal peptide, e.g., as disclosed herein. In some embodiments, a signal peptide is chosen from: SEQ ID NO: 39, SEQ ID NO: 6, SEQ ID NO: 38, SEQ ID NO: 35 or SEQ ID NO: 36.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 45. In some embodiments, a first moiety comprises one or more peptides comprising the sequence of SEQ ID NO: 45.

In some embodiments, a first moiety comprises one or more peptides comprising a sequence having at least 85% identity to SEQ ID NO: 45 and a signal peptide, e.g., as disclosed herein.

In some embodiments, a neutrophil autoantigen is a PR3 polypeptide or a variant or fragment thereof. In some embodiments, a PR3 polypeptide variant or fragment exhibits reduced or eliminated protease activity relative to a full-length, wild-type PR3 polypeptide.

In some embodiments, a neutrophil autoantigen is a PR3 polypeptide comprising a mutation at one or more of amino acids 71 (His), 118 (Asp) and 203 (Ser) of SEQ ID NO: 1.

In some embodiments, a neutrophil autoantigen is a PR3 polypeptide comprising a mutation at one or more of amino acids 180 (Phe), 181 (Phe), 228 (Leu), or 229 (Phe) of SEQ ID NO: 1.

In some embodiments, an anti-neutrophil autoantibody is an anti-PR3 autoantibody or a fragment thereof. In some embodiments, an anti-PR3 autoantibody is characterized in that it binds to a PR3 polypeptide or a variant or fragment thereof.

In some embodiments, a first moiety of a glycoengineered polypeptide disclosed herein comprises one or more peptides that specifically bind to an anti-PR3 autoantibody. In some embodiments, a one or more polypeptides that specifically bind to an anti-PR3 autoantibody comprises a PR3 polypeptide, or a fragment or a variant thereof.

In some embodiments, one or more polypeptides that specifically bind to an anti-PR3 autoantibody comprises a soluble fragment of a PR3 polypeptide.

In some embodiments, one or more polypeptides that specifically bind to an anti-PR3 autoantibody comprises a fragment of a PR3 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PR3 polypeptide of SEQ ID NO: 1.

In some embodiments, one or more polypeptides that specifically bind to an anti-PR3 autoantibody comprises a fragment of a PR3 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PR3 polypeptide of SEQ ID NO: 1 without the signal peptide.

In some embodiments, one or more polypeptides that specifically bind to an anti-PR3 autoantibody comprises a fragment of a PR3 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PR3 polypeptide of SEQ ID NO: 1.

In some embodiments, one or more polypeptides that specifically bind to an anti-PR3 autoantibody comprises a fragment of a PR3 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PR3 polypeptide of SEQ ID NO: 1 without the signal peptide.

In some embodiments, one or more polypeptides that specifically bind to an anti-PR3 autoantibody comprises a fragment of a PR3 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PR3 polypeptide of SEQ ID NO: 40.

In some embodiments, one or more polypeptides that specifically bind to an anti-PR3 autoantibody comprises a fragment of a PR3 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PR3 polypeptide of SEQ ID NO: 40 without the signal peptide.

In some embodiments, one or more polypeptides that specifically bind to an anti-PR3 autoantibody comprises a fragment of a PR3 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PR3 polypeptide of SEQ ID NO: 40.

In some embodiments, one or more polypeptides that specifically bind to an anti-PR3 autoantibody comprises a fragment of a PR3 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PR3 polypeptide of SEQ ID NO: 40 without the signal peptide.

In some embodiments, one or more polypeptides that specifically bind to an anti-PR3 autoantibody comprises a fragment of a PR3 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PR3 polypeptide of SEQ ID NO: 45.

In some embodiments, one or more polypeptides that specifically bind to an anti-PR3 autoantibody comprises a fragment of a PR3 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PR3 polypeptide of SEQ ID NO: 45.

In some embodiments, a fragment comprises an epitope that is recognized by a PR3 autoantibody. In some embodiments, an epitope is a linear epitope. In some embodiments, an epitope is a conformational epitope. In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by an anti-neutrophil autoantibody, and X denotes intervening amino acid sequences with n being an integer from 0-20.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn-[An]-Xn, wherein A1 is a first portion of an epitope, A2 is a second portion of an epitope, and An is the n-th portion of an epitope which together form a spatial epitope that is recognized by an anti-neutrophil autoantibody, and X denotes intervening amino acid sequences with n being an integer from 0-20.

In some embodiments, a first moiety comprises a plurality of epitopes, e.g., the same or different epitopes. In some embodiments, a first moiety comprises a plurality of the same epitopes, e.g., epitopes recognized by an anti-PR3 autoantibody. In some embodiments, a first moiety comprises a plurality of different epitopes, e.g., epitopes recognized by different anti-PR3 autoantibodies. In some embodiments, the plurality of epitopes is separated by a linker, IRES or cleavage peptide.

PR3 epitopes that may be recognized by anti-PR3 autoantibodies are disclosed in Van Der Geld Y M et al., (2004) Clin Exp Immunol, Vol 137, pp. 451-459, the entire contents of which are hereby incorporated by reference. In some embodiments, one or more epitopes of a first moiety disclosed herein comprises an epitope disclosed in Van Der Geld et al., 2004.

In some embodiments, an epitope that is recognized by a PR3 autoantibody comprises IVGGHEAQPHSRPYMASLOMR (SEQ ID NO: 2). In some embodiments, an epitope comprises one or more additional sequences 5′ and/or 3′ to the epitope sequence.

In some embodiments, an epitope that is recognized by a PR3 autoantibody comprises IVGGHEAQPHSRPYMASLOM (SEQ ID NO: 3). In some embodiments, an epitope comprises one or more additional sequences 5′ and/or 3′ to the epitope sequence.

In some embodiments, an epitope that is recognized by a PR3 autoantibody comprises GHEAQPHSRPY (SEQ ID NO: 11). In some embodiments, an epitope comprises one or more additional sequences 5′ and/or 3′ to the epitope sequence.

In some embodiments, an epitope that is recognized by a PR3 autoantibody comprises TQEPTQQ (SEQ ID NO. 12). In some embodiments, an epitope comprises one or more additional sequences 5′ and/or 3′ to the epitope sequence.

In some embodiments, an epitope that is recognized by a PR3 autoantibody comprises ATVQLPQQDQPVPHGTQ (SEQ ID NO. 13). In some embodiments, an epitope comprises one or more additional sequences 5′ and/or 3′ to the epitope sequence.

In some embodiments, an epitope that is recognized by a PR3 autoantibody comprises RVGAHDPP (SEQ ID NO. 14). In some embodiments, an epitope comprises one or more additional sequences 5′ and/or 3′ to the epitope sequence.

In some embodiments, an epitope that is recognized by a PR3 autoantibody comprises FCRPHNI (SEQ ID NO. 15). In some embodiments, an epitope comprises one or more additional sequences 5′ and/or 3′ to the epitope sequence.

In some embodiments, an epitope that is recognized by a PR3 autoantibody comprises PRRKAGICFGDSGGP (SEQ ID NO. 16). In some embodiments, an epitope comprises one or more additional sequences 5′ and/or 3′ to the epitope sequence.

In some embodiments, an epitope that is recognized by a PR3 autoantibody comprises IDSFVIW (SEQ ID NO. 17). In some embodiments, an epitope comprises one or more additional sequences 5′ and/or 3′ to the epitope sequence.

In some embodiments, an epitope that is recognized by a PR3 autoantibody comprises PYMASL (SEQ ID NO. 18). In some embodiments, an epitope comprises one or more additional sequences 5′ and/or 3′ to the epitope sequence.

In some embodiments, an epitope that is recognized by a PR3 autoantibody comprises AHCLRDIPQRLVNV (SEQ ID NO. 19). In some embodiments, an epitope comprises one or more additional sequences 5′ and/or 3′ to the epitope sequence.

In some embodiments, an epitope that is recognized by a PR3 autoantibody comprises HGTQCLAMGWGRVGAH (SEQ ID NO. 20). In some embodiments, an epitope comprises one or more additional sequences 5′ and/or 3′ to the epitope sequence.

In some embodiments, an epitope that is recognized by a PR3 autoantibody comprises NICTFVPRRKAGIC (SEQ ID NO. 21). In some embodiments, an epitope comprises one or more additional sequences 5′ and/or 3′ to the epitope sequence.

In some embodiments, an epitope that is recognized by a PR3 autoantibody comprises CATRLFPDFFTRVAL (SEQ ID NO. 22). In some embodiments, an epitope comprises one or more additional sequences 5′ and/or 3′ to the epitope sequence.

In some embodiments, an epitope that is recognized by a PR3 autoantibody comprises WIRSTRLLVEAKGRP (SEQ ID NO. 23). In some embodiments, an epitope comprises one or more additional sequences 5′ and/or 3′ to the epitope sequence.

In some embodiments, one or more polypeptides that specifically bind to an anti-PR3 autoantibody comprises a variant of a PR3 polypeptide. In some embodiments, a variant is an inactive variant as compared to a wild-type PR3 polypeptide.

In some embodiments, a variant comprises a mutation at the Valine residue at position 119, the Alanine residue at position 135, the Threonine residue at position 136, or a combination thereof.

In some embodiments, a variant comprises a mutation at one or more or all of amino acids 71 (His), 118 (Asp) and 203 (Ser) of SEQ ID NO: 1.

In some embodiments, a variant comprises a mutation at amino acid 71 (His), of SEQ ID NO: 1. In some embodiments, a variant comprising a mutation at amino acid 71 (His), further comprises one or more additional mutations to SEQ ID NO: 1, e.g., as described herein.

In some embodiments, a variant comprises a mutation at amino acid 118 (Asp) of SEQ ID NO: 1. In some embodiments, a variant comprising a mutation at amino acid 118 (Asp) further comprises one or more additional mutations to SEQ ID NO: 1, e.g., as described herein.

In some embodiments, a variant comprises a mutation at amino acid 203 (Ser) of SEQ ID NO: 1. In some embodiments, a variant comprises a Serine to Alanine mutation at position 203 of SEQ ID NO: 1. In some embodiments, a variant comprising a mutation at amino acid 203 (Ser) further comprises one or more additional mutations to SEQ ID NO: 1, e.g., as described herein.

In some embodiments, a variant comprises a mutation at amino acids 71 (His), 118 (Asp) and 203 (Ser) of SEQ ID NO: 1. In some embodiments, a variant comprising a mutation at amino acids 71 (His), 118 (Asp) and 203 (Ser) of SEQ ID NO: 1 further comprises one or more additional mutations to SEQ ID NO: 1, e.g., as described herein.

In some embodiments, a variant comprises a mutation at one or more or all of amino acids 180 (Phe), 181 (Phe), 228 (Leu), or 229 (Phe) of SEQ ID NO: 1.

In some embodiments, a variant comprises a mutation at amino acid 180 (Phe) of SEQ ID NO: 1. In some embodiments, a variant comprises a Phenylalanine to Alanine mutation at position 180 of SEQ ID NO: 1. In some embodiments, a variant comprising a mutation at amino acid 180 (Phe) further comprises one or more additional mutations to SEQ ID NO: 1, e.g., as described herein.

In some embodiments, a variant comprises a mutation at amino acid 181 (Phe) of SEQ ID NO: 1. In some embodiments, a variant comprises a Phenylalanine to Alanine mutation at position 181 of SEQ ID NO: 1. In some embodiments, a variant comprising a mutation at amino acid 181 (Phe) further comprises one or more additional mutations to SEQ ID NO: 1, e.g., as described herein.

In some embodiments, a variant comprises a mutation at amino acid 228 (Leu) of SEQ ID NO: 1. In some embodiments, a variant comprises a Leucine to Alanine mutation at position 228 of SEQ ID NO: 1. In some embodiments, a variant comprising a mutation at amino acid 228 (Leu) further comprises one or more additional mutations to SEQ ID NO: 1, e.g., as described herein.

In some embodiments, a variant comprises a mutation at amino acid 229 (Phe) of SEQ ID NO: 1. In some embodiments, a variant comprises a Phenylalanine to Alanine mutation at position 229 of SEQ ID NO: 1. In some embodiments, a variant comprising a mutation at amino acid 229 (Phe) further comprises one or more additional mutations to SEQ ID NO: 1, e.g., as described herein.

In some embodiments, a variant comprises a mutation at amino acids 180 (Phe), 181 (Phe), 228 (Leu), or 229 (Phe) of SEQ ID NO: 1. In some embodiments, a variant comprising a mutation, e.g., into an Alanine, at amino acids 180 (Phe), 181 (Phe), 228 (Leu), or 229 (Phe) of SEQ ID NO: 1 further comprises one or more additional mutations to SEQ ID NO: 1, e.g., as described herein.

In some embodiments, a variant comprises a PR3 polypeptide or a fragment thereof having one or more mutations at a glycosylation site. In some embodiments, a mutation is at an Asparagine residue such that a glycosylation site is altered. In some embodiments, a mutation comprises a mutation at Asparagine 129 and/or Asparagine 174.

In some embodiments, a mutation is a Valine to Isoleucine mutation. In some embodiments, a mutation is an Alanine to Threonine mutation. In some embodiments, a mutation is a Serine to Threonine mutation.

In some embodiments, a glycoengineered polypeptide comprises a PR3 polypeptide or a fragment thereof having one or more native glycosylation sites.

In some embodiments, a glycoengineered polypeptide comprises a PR3 polypeptide or a fragment thereof having two native glycosylation sites. In some embodiments, a native glycosylation site comprises N129 or N174.

In some embodiments, a glycoengineered polypeptide comprises a PR3 polypeptide or a fragment thereof having one or more engineered glycosylation sites. In some embodiments, an engineered glycosylation site is or comprises the sequence of GGGGANSTAPAPAPA (SEQ ID NO: 37).

In some embodiments, a glycoengineered polypeptide comprises a PR3 polypeptide or a fragment thereof having two native glycosylation sites N129 and N174 and one engineered glycosylation site having the sequence GGGGANSTAPAPAPA (SEQ ID NO: 37).

In some embodiments, a glycoengineered polypeptide further comprises one or more additional elements. In some embodiments, one or more additional elements comprise: (a) a linker, (b) a spacer, (c) a cleavage peptide, e.g., an IRES or a protease cleavage site, (d) a signal peptide, (e) a tag, e.g., a cleavable tag, (f) a half-life extender domain, e.g., an Fc domain or albumin, or (g) any combination of (a)-(f).

In some embodiments, a glycoengineered polypeptide comprises a tag, e.g., a His tag.

In some embodiments, a glycoengineered polypeptide comprises the following sequence (SEQ ID NO: 41):

MAHRPPSPALASVLLALLLSGAARAIVGGHEAQPHSRPYMASLQMRG
NPGSHFCGGTLIHPSFVLTAAHCLRDIPQRLVNVVLGAHNVRTQEPTQQHFSVAQVFLN
NYDAENKLNDVLLIQLSSPANLSASVATVQLPQQDQPVPHGTQCLAMGWGRVGAHDP
PAQVLQELNVTVVTAACRPHNICTFVPRRKAGICFGDAGGPLICDGIIQGIDSFVIWGCA
TRAAPDFFTRVALYVDWIRSTLRRVEAKGRPGGGGANSTAPAPAPAHHHHHHHHHH

In some embodiments, a glycoengineered polypeptide comprises a sequence with at least 85%, at least 90%, at least 95%, at least 99% identity to SEQ ID NO: 41. In some embodiments, a glycoengineered polypeptide comprises the sequence of SEQ ID NO: 41.

In some embodiments, one or more polypeptides that specifically bind to an anti-PR3 autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 1.

In some embodiments, one or more polypeptides that specifically bind to an anti-PR3 autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 41.

In some embodiments, one or more polypeptides that specifically bind to an anti-PR3 autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 45.

In some embodiments, one or more polypeptides that specifically bind to an anti-PR3 autoantibody comprises a full length PR3 protein, e.g., as provided in SEQ ID NO: 1 with or without the signal peptide.

In some embodiments, one or more polypeptides that specifically bind to an anti-PR3 autoantibody comprises a PR3 protein, e.g., as provided in SEQ ID NO: 45.

In some embodiments, a first moiety comprises 1, 2, 3, 4, 5, or more peptides that specifically bind to an anti-PR3 autoantibody.

In some embodiments, one or more peptides of a first moiety that specifically bind to an anti-PR3 autoantibody are the same, e.g., the one or more peptides have the same sequence of a PR3 polypeptide, or a fragment or a variant thereof. In some embodiments, the one or more peptides having the same sequence of a PR3 polypeptide, or a fragment or a variant thereof are separated by one or more intervening sequences (e.g., spacers and/or linkers). In some embodiments, the one or more peptides having the same sequence of a PR3 polypeptide, or a fragment or a variant thereof are not separated by one or more intervening sequences (e.g., spacers and/or linkers).

In some embodiments, one or more peptides of a first moiety that specifically bind to an anti-PR3 autoantibody are different, e.g., the one or more peptides do not have the same sequence of a PR3 polypeptide, or a fragment or a variant thereof. In some embodiments, the one or more peptides having different sequences of a PR3 polypeptide, or a fragment or a variant thereof are separated by one or more intervening sequences (e.g., spacers and/or linkers). In some embodiments, the one or more peptides different sequences of a PR3 polypeptide, or a fragment or a variant thereof are not separated by one or more intervening sequences (e.g., spacers and/or linkers).

In some embodiments, each of the one or more peptides having different sequences of a PR3 polypeptide, or a fragment or a variant thereof specifically bind to an anti-PR3 autoantibody or a fragment thereof.

In some embodiments, a linker separating one or more peptides of a first moiety comprises a Gly-Ser linker, or an EAAAK linker. In some embodiments, a linker comprises a (Gly-Gly-Gly-Gly-Ser) n linker, wherein n is an integer between 0 to 20.

In some embodiments, one or more peptides that specifically bind to an anti-PR3 autoantibody are each conjugated to a second moiety.

In some embodiments, one or more peptides that specifically bind to an anti-PR3 autoantibody are not each conjugated to the second moiety.

In some embodiments, one or more peptides that specifically bind to an anti-PR3 autoantibody are conjugated to each other, e.g., are situated on one polypeptide.

In some embodiments, one or more peptides that specifically bind to an anti-PR3 autoantibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an anti-PR3 autoantibody are not separated by a protease cleavage site or an IRES, e.g., is expressed as a fusion protein.

In some embodiments, a first moiety comprises one or more peptides that specifically binds to an anti-PR3 antibody. In some embodiments, one or more peptides that specifically bind to an anti-PR3 antibody comprise a PR3 polypeptide, or a variant or a fragment thereof. In some embodiments, a first moiety further comprises: (a) a linker, (b) a spacer, (c) a cleavage peptide, e.g., an IRES or, a protease cleavage site, (d) a signal peptide, (e) a tag, e.g., a cleavable tag, (f) a half-life extender domain, e.g., an Fc domain or albumin, (g) any combination of (a)-(f).

In some embodiments, one or more peptides that specifically bind to an anti-MPO antibody comprise an antibody agent. In some embodiments, the antibody agent comprises an antigen binding fragment. In some embodiments, the antibody agent comprises a full antibody, a Fab fragment, an scFv, a nanobody, a duobody, a single domain antibody (e.g., a VHH). In some embodiments, the antibody agent comprises a VHH, e.g., a camelid VHH or a bivalent VHH.

### Myeloperoxidase (MPO) and First Moiety Comprising MPO Peptides

MPO is heme-containing peroxidase expressed mainly in neutrophils and monocytes. MPO catalyzes the formation of reactive oxygen intermediates in the presence of hydrogen peroxide and halides (Aratani Y., Arch Biochem Biophys. 2018 Feb. 15; 640:47-52.). Typically, transcription of MPO is turned off before neutrophils are released from the bone marrow, however in ANCA vasculitis increased and/or aberrant expression of MPO has been reported. Additionally, ANCA vasculitis has also been shown to be associated with increased levels of MPO on the surface of circulating neutrophils (Jennette C. J. et al., Curr Opin Nephrol Hypertens (2011) 20(3): 263-270).

A human MPO polypeptide sequence is provided herein as SEQ ID NO: 4, with the bolded sequence denoting the signal peptide (corresponding to UniProt Accession number P05164):

| MGVPFFSSLRCMVDLGPCWAGGLTAEMKLLLALAGLLAILATPQ | |
|---|---|
| PSEGAAPAVLGEVDTSLVLSSMEEAKQLVDKAYKERRESIKQRLRSGSASPMELLSYF | |
| KQPVAATRTAVRAADYLHVALDLLERKLRSLWRRPFNVTDVLTPAQLNVLSKSSGCA | |
| YQDVGVTCPEQDKYRTITGMCNNRRSPTLGASNRAFVRWLPAEYEDGFSLPYGWTPG | |
| VKRNGFPVALARAVSNEIVRFPTDQLTPDQERSLMFMQWGQLLDHDLDFTPEPAARAS | |
| FVTGVNCETSCVQQPPCFPLKIPPNDPRIKNQADCIPFFRSCPACPGSNITIRNQINALTSF | |
| VDASMVYGSEEPLARNLRNMSNQLGLLAVNQRFQDNGRALLPFDNLHDDPCLLTNRS | |
| ARIPCFLAGDTRSSEMPELTSMHTLLLREHNRLATELKSLNPRWDGERLYQEARKIVGA | |
| MVQIITYRDYLPLVLGPTAMRKYLPTYRSYNDSVDPRIANVFTNAFRYGHTLIQPFMFR | |
| LDNRYQPMEPNPRVPLSRVFFASWRVVLEGGIDPILRGLMATPAKLNRQNQIAVDEIRE | |
| RLFEQVMRIGLDLPALNMQRSRDHGLPGYNAWRRFCGLPQPETVGQLGTVLRNLKLA | |
| RKLMEQYGTPNNIDIWMGGVSEPLKRKGRVGPLLACIIGTQFRKLRDGDRFWWENEG | |
| VFSMQQRQALAQISLPRIICDNTGITTVSKNNIFMSNSYPRDFVNCSTLPALNLASWREA | |
| S | |
| Human MPO signal peptide: | |
| (SEQ ID NO: 8) | |
| MGVPFFSSLRCMVDLGPCWAGGLTAEMKLLLALAGLLAILATPQPSEG | |
| Human MPO can be encoded by the following nucleic acid sequence from the | |
| myeloperoxidase gene | |
| (SEQ ID NO: 9) | |
| 1 | gtccttggaa gctggatgac agcagctggc aaggggataa gagagcagtg agcccctccc |
| 61 | tcaaggaggt ctggctttat ccatagacag ggccctctga ggtggggctg aggtacaaag |
| 121 | ggggattgag cagcccagga gaagagagat gggggttccc ttcttctctt ctctcagatg |
| 181 | catggtggac ttaggacctt gctgggctgg gggtctcact gcagagatga agctgcttct |
| 241 | ggccctagca gggctcctgg ccattctggc cacgccccag ccctctgaag gtgctgctcc |
| 301 | agctgtcctg ggggaggtgg acacctcgtt ggtgctgagc tccatggagg aggccaagca |
| 361 | gctggtggac aaggcctaca aggagcggcg ggaaagcatc aagcagcggc ttcgcagcgg |
| 421 | ctcagccagc cccatggaac tcctatccta cttcaagcag ccggtggcag ccaccaggac |
| 481 | ggcggtgagg gccgctgact acctgcacgt ggctctagac ctgctggaga ggaagctgcg |
| 541 | gtccctgtgg cgaaggccat tcaatgtcac tgatgtgctg acgcccgccc agctgaatgt |
| 601 | gttgtccaag tcaagcggct gcgcctacca ggacgtgggg gtgacttgcc cggagcagga |
| 661 | caaataccgc accatcaccg ggatgtgcaa caacagacgc agccccacgc tgggggcctc |
| 721 | caaccgtgcc tttgtgcgct ggctgccggc ggagtatgag gacggcttct ctcttcccta |
| 781 | cggctggacg cccggggtca agcgcaacgg cttcccggtg gctctggctc gcgcggtctc |
| 841 | caacgagatc gtgcgcttcc ccactgatca gctgactccg gaccaggagc gctcactcat |
| 901 | gttcatgcaa tggggccagc tgttggacca cgacctcgac ttcacccctg agccggccgc |
| 961 | ccgggcctcc ttcgtcactg gcgtcaactg cgagaccagc tgcgttcagc agccgccctg |
| 1021 | cttcccgctc aagatcccgc ccaatgaccc ccgcatcaag aaccaagccg actgcatccc |
| 1081 | gttcttccgc tcctgcccgg cttgccccgg gagcaacatc accatccgca accagatcaa |
| 1141 | cgcgctcact tccttcgtgg acgccagcat ggtgtacggc agcgaggagc ccctggccag |
| 1201 | gaacctgcgc aacatgtcca accagctggg gctgctggcc gtcaaccagc gcttccaaga |
| 1261 | caacggccgg gccctgctgc cctttgacaa cctgcacgat gacccctgtc tcctcaccaa |
| 1321 | ccgctcagcg cgcatcccct gcttcctggc aggggacacc cgttccagtg agatgcccga |
| 1381 | gctcacctcc atgcacaccc tcttacttcg ggagcacaac cggctggcca cagagctcaa |
| 1441 | gagcctgaac cctaggtggg atggggagag gctctaccag gaagcccgga agatcgtggg |
| 1501 | ggccatggtc cagatcatca cttaccggga ctacctgccc ctggtgctgg ggccaacggc |
| 1561 | catgaggaag tacctgccca cgtaccgttc ctacaatgac tcagtggacc cacgcatcgc |
| 1621 | caacgtcttc accaatgcct tccgctacgg ccacaccctc atccaaccct tcatgttccg |
| 1681 | cctggacaat cggtaccagc ccatggaacc caacccccgt gtccccctca gcagggtctt |
| 1741 | ttttgcctcc tggagggtcg tgctggaagg tggcattgac cccatcctcc ggggcctcat |
| 1801 | ggccacccct gccaagctga atcgtcagaa ccaaattgca gtggatgaga tccgggagcg |
| 1861 | attgtttgag caggtcatga ggattgggct ggacctgcct gctctgaaca tgcagcgcag |
| 1921 | cagggaccac ggcctcccag gatacaatgc ctggaggcgc ttctgtgggc tcccgcagcc |
| 1981 | tgaaactgtg ggccagctgg gcacggtgct gaggaacctg aaattggcga ggaaactgat |
| 2041 | ggagcagtat ggcacgccca acaacatcga catctggatg ggcggcgtgt ccgagcctct |
| 2101 | gaagcgcaaa ggccgcgtgg gcccactcct cgcctgcatc atcggtaccc agttcaggaa |
| 2161 | gctccgggat ggtgatcggt tttggtggga gaacgagggt gtgttcagca tgcagcagcg |
| 2221 | acaggccctg gcccagatct cattgccccg gatcatctgc gacaacacag gcatcaccac |
| 2281 | cgtgtctaag aacaacatct tcatgtccaa ctcatatccc cgggactttg tcaactgcag |
| 2341 | tacacttcct gcattgaacc tggcttcctg gagggaagcc tcctagaggc caggtaaggg |
| 2401 | ggtgcagcag tgaggggtat atctgggctg gccagttgga accacggaga tctccttgcc |
| 2461 | ctagatgagc ccagccctgt tctgggtgca gctgagaaaa tgagtgacta gacgttcatt |
| 2521 | tgtgtgctca tgtatgtgcg aagtatataa attggctttt catgcgtg |

In some embodiments, an MPO polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 4. In some embodiments, an MPO polypeptide is or comprises SEQ ID NO: 4. In some embodiments, an MPO polypeptide is or comprises SEQ ID NO: 4 without the signal peptide of SEQ ID NO: 8.

In some embodiments, an MPO polypeptide comprising a sequence having at least 85% identity to SEQ ID NO: 4 without the signal peptide of SEQ ID NO: 8, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 4. In some embodiments, a first moiety comprises one or more peptides comprising the sequence of SEQ ID NO: 4. In some embodiments, a first moiety comprises one or more peptides comprising the sequence of SEQ ID NO: 4 without the signal peptide of SEQ ID NO: 8.

In some embodiments, a first moiety comprises one or more peptides comprising a sequence having at least 85% identity to SEQ ID NO: 4 without the signal peptide of SEQ ID NO: 8.

In some embodiments, a first moiety comprising one or more peptides comprising a sequence having at least 85% identity to SEQ ID NO: 4 without the signal peptide of SEQ ID NO: 8, further comprises a different signal peptide, e.g., as disclosed herein.

An exemplary engineered MPO polypeptide sequence is provided herein as SEQ ID NO: 42, with the bolded sequence denoting the signal peptide:

MASRLVRVLAAAMLVAAAVSAAPAVLGEVDTSLVLSSMEEAKQLV
DKAYKERRESIKQRLRSGSASPMELLSYFKQPVAATRTAVRAADYLHVALDLLERKLR
SLWRRPFNVTDVLTPAQLNVLSKSSGCAYQDVGVTCPEQDKYRTITGMCNNRRSPTLG
ASNRAFVRWLPAEYEDGFSLPYGWTPGVKRNGFPVALARAVSNEIVRFPTDQLTPDQE
RSLMFMQWGQLLDADLDFTPEPAARASFVTGVNCETSCVQQPPCFPLKIPPNDPRIKNQ
ADCIPFFRSAPACPGSNITIRNQINALTSFVDASMVYGSEEPLARNLRNMSNQLGLLAVN
QRFQDNGRALLPFDNLHDDPCLLTNRSARIPCFLAGDTRSSEMPELTSMHTLLLREHNR
LATELKSLNPRWDGERLYQEARKIVGAMVQIITYRDYLPLVLGPTAMRKYLPTYRSYN
DSVDPRIANVFTNAFRYGHTLIQPFMFRLDNRYQPMEPNPRVPLSRVFFASWRVVLEG
GIDPILRGLMATPAKLNRQNQIAVDEIRERLFEQVMRIGLDLPALNMQRSRDHGLPGYN
AWRRFCGLPQPETVGQLGTVLRNLKLARKLMEQYGTPNNIDIWMGGVSEPLKRKGRV
GPLLACIIGTQFRKLRDGDRFWWENEGVFSMQQRQALAQISLPRIICDNTGITTVSKNNI
FMSNSYPRDFVNCSTLPALNLASWREAS
Leishmania signal peptide:
(SEQ ID NO: 38)
MASRLVRVLAAAMLVAAAVS

In some embodiments, an MPO polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 42. In some embodiments, an MPO polypeptide is or comprises SEQ ID NO: 42. In some embodiments, an MPO polypeptide is or comprises SEQ ID NO: 42 without the signal peptide of SEQ ID NO: 38.

In some embodiments, an MPO polypeptide comprises a sequence having at least 85% identity to SEQ ID NO: 42 without the signal peptide of SEQ ID NO: 38.

In some embodiments, an MPO polypeptide comprising a sequence having at least 85% identity to SEQ ID NO: 42 without the signal peptide of SEQ ID NO: 38, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 42. In some embodiments, a first moiety comprises one or more peptides comprising the sequence of SEQ ID NO: 42. In some embodiments, a first moiety comprises one or more peptides comprising the sequence of SEQ ID NO: 42 without the signal peptide of SEQ ID NO: 38.

In some embodiments, a first moiety comprises one or more peptides comprising a sequence having at least 85% identity to SEQ ID NO: 42 without the signal peptide of SEQ ID NO: 38.

In some embodiments, a first moiety comprising one or more peptides comprising a sequence having at least 85% identity to SEQ ID NO: 42 without the signal peptide of SEQ ID NO: 38, further comprises a different signal peptide, e.g., as disclosed herein.

An exemplary MPO polypeptide without a signal peptide is provided herein as SEQ ID NO: 46:

AAPAVLGEVDTSLVLSSMEEAKQLVDKAYKERRESIKQRLRSGSASP
MELLSYFKQPVAATRTAVRAADYLHVALDLLERKLRSLWRRPFNVTDVLTPAQLNVL
SKSSGCAYQDVGVTCPEQDKYRTITGMCNNRRSPTLGASNRAFVRWLPAEYEDGFSLP
YGWTPGVKRNGFPVALARAVSNEIVRFPTDQLTPDQERSLMFMQWGQLLDADLDFTP
EPAARASFVTGVNCETSCVQQPPCFPLKIPPNDPRIKNQADCIPFFRSAPACPGSNITIRN
QINALTSFVDASMVYGSEEPLARNLRNMSNQLGLLAVNQRFQDNGRALLPFDNLHDD
PCLLTNRSARIPCFLAGDTRSSEMPELTSMHTLLLREHNRLATELKSLNPRWDGERLYQ
EARKIVGAMVQIITYRDYLPLVLGPTAMRKYLPTYRSYNDSVDPRIANVFTNAFRYGH
TLIQPFMFRLDNRYQPMEPNPRVPLSRVFFASWRVVLEGGIDPILRGLMATPAKLNRQN
QIAVDEIRERLFEQVMRIGLDLPALNMQRSRDHGLPGYNAWRRFCGLPQPETVGQLGT
VLRNLKLARKLMEQYGTPNNIDIWMGGVSEPLKRKGRVGPLLACIIGTQFRKLRDGDR
FWWENEGVFSMQQRQALAQISLPRIICDNTGITTVSKNNIFMSNSYPRDFVNCSTLPAL
NLASWREAS

In some embodiments, an MPO polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 46. In some embodiments, an MPO polypeptide is or comprises SEQ ID NO: 46.

In some embodiments, an MPO polypeptide comprising an amino acid having at least 85% identity to SEQ ID NO: 46 further comprises a signal peptide disclosed herein. In some embodiments, a signal peptide is a Leishmania-derived signal peptide.

In some embodiments, a signal peptide is chosen from: SEQ ID NO: 8 SEQ ID NO: 35, SEQ ID NO: 36, or SEQ ID NO: 38.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 46. In some embodiments, a first moiety comprises one or more peptides comprising the sequence of SEQ ID NO: 46.

In some embodiments, a first moiety comprises one or more peptides comprising a sequence having at least 85% identity to SEQ ID NO: 46 and a signal peptide, e.g., as disclosed herein.

In some embodiments, a neutrophil autoantigen is an MPO polypeptide or a variant or a fragment thereof. In some embodiments, an MPO polypeptide variant or fragment exhibits reduced or eliminated protease activity relative to a full-length, wild-type MPO polypeptide.

In some embodiments, a neutrophil autoantigen is an MPO polypeptide comprising a mutation at one or more of amino acids 261 (His), 316 (Cys), 405 (Arg) and 257 (Gln) of SEQ ID NO: 4.

In some embodiments, an anti-neutrophil autoantibody is an MPO autoantibody or a fragment thereof. In some embodiments, an anti-MPO autoantibody is characterized in that it binds to an MPO polypeptide or a variant or a fragment thereof.

In some embodiments, a first moiety of a glycoengineered polypeptide disclosed herein comprises one or more peptides that specifically bind to an anti-MPO autoantibody. In some embodiments, a one or more polypeptides that specifically bind to an anti-MPO autoantibody comprises a MPO polypeptide, or a fragment or a variant thereof.

In some embodiments, one or more polypeptides that specifically bind to an anti-MPO autoantibody comprises a soluble fragment of a MPO polypeptide.

In some embodiments, one or more polypeptides that specifically bind to an anti-MPO autoantibody comprises a fragment of a MPO polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length MPO polypeptide of SEQ ID NO: 4.

In some embodiments, one or more polypeptides that specifically bind to an anti-MPO autoantibody comprises a fragment of a MPO polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length MPO polypeptide of SEQ ID NO: 4 without the signal peptide.

In some embodiments, one or more polypeptides that specifically bind to an anti-MPO autoantibody comprises a fragment of a MPO polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length MPO polypeptide of SEQ ID NO: 4.

In some embodiments, one or more polypeptides that specifically bind to an anti-MPO autoantibody comprises a fragment of a MPO polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length MPO polypeptide of SEQ ID NO: 4 without the signal peptide.

In some embodiments, one or more polypeptides that specifically bind to an anti-MPO autoantibody comprises a fragment of a MPO polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length MPO polypeptide of SEQ ID NO: 42.

In some embodiments, one or more polypeptides that specifically bind to an anti-MPO autoantibody comprises a fragment of a MPO polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length MPO polypeptide of SEQ ID NO: 42 without the signal peptide.

In some embodiments, one or more polypeptides that specifically bind to an anti-MPO autoantibody comprises a fragment of a MPO polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length MPO polypeptide of SEQ ID NO: 42.

In some embodiments, one or more polypeptides that specifically bind to an anti-MPO autoantibody comprises a fragment of a MPO polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length MPO polypeptide of SEQ ID NO: 42 without the signal peptide.

In some embodiments, one or more polypeptides that specifically bind to an anti-MPO autoantibody comprises a fragment of a MPO polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length MPO polypeptide of SEQ ID NO: 46.

In some embodiments, one or more polypeptides that specifically bind to an anti-MPO autoantibody comprises a fragment of a MPO polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length MPO polypeptide of SEQ ID NO: 46.

In some embodiments, a fragment comprises an epitope that is recognized by a MPO autoantibody. In some embodiments, an epitope is a linear epitope. In some embodiments, an epitope is a conformational epitope. In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by an anti-neutrophil autoantibody, and X denotes intervening amino acid sequences with n being an integer from 0-20.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn . . . [An]-Xn, wherein A1 is a first portion of an epitope, A2 is a second portion of an epitope, and An is the n-th portion of an epitope which together form a spatial epitope that is recognized by an anti-neutrophil autoantibody, and X denotes intervening amino acid sequences with n being an integer from 0-20.

In some embodiments, a first moiety comprises a plurality of epitopes, e.g., the same or different epitopes. In some embodiments, a first moiety comprises a plurality of the same epitopes, e.g., epitopes recognized by an anti-MPO autoantibody. In some embodiments, a first moiety comprises a plurality of different epitopes, e.g., epitopes recognized by different anti-MPO autoantibodies. In some embodiments, the plurality of epitopes is separated by a linker, IRES or cleavage peptide.

MPO epitopes that can be recognized by anti-MPO autoantibodies are disclosed in Bruner B F et al., (2011) Clin Exp Immunol., volume 164, pp. 330-336, the entire contents of which are here by incorporated by reference. In some embodiments, one or more epitopes of a first moiety disclosed herein comprises an epitope disclosed in Bruner et al., 2011.

MPO epitopes that can be recognized by anti-MPO autoantibodies are disclosed in Van der Geld et al. (2004) Clin Exp Immunol 137:451-459, the entire contents of which are here by incorporated by reference. In some embodiments, one or more epitopes of a first moiety disclosed herein comprises an epitope disclosed in Van Der Geld et al., 2004.

MPO epitopes that can be recognized by anti-MPO autoantibodies are disclosed in Free et al. (2020) J. Immunol. 106:102306; and 447-461, the entire contents of which are here by incorporated by reference. In some embodiments, one or more epitopes of a first moiety disclosed herein comprises an epitope disclosed in Free et al., 2020.

In some embodiments, an epitope that is recognized by a MPO autoantibody comprises PRWDGERLYQEARKIVGAMV (SEQ ID NO: 5). In some embodiments, an epitope comprises one or more additional sequences 5′ and/or 3′ to the epitope sequence.

In some embodiments, an epitope that is recognized by a MPO autoantibody comprises RLYQEARKIVG (SEQ ID NO: 10). In some embodiments, an epitope comprises one or more additional sequences 5′ and/or 3′ to the epitope sequence.

In some embodiments, an epitope that is recognized by a MPO autoantibody comprises VNCETSCVQQPPCFPLKIPPNDPRIKNQADCIPFFRSCPACP GSNITIRNQI NALTSFVDASMVYGSEEPLARNLRNMSNQLGLLAVNQRFQDNGRALLPFD NLHDDPCLLTNRSARIPCFLAGDTRSSEMPELTSMHTLLLREHNRLATEL KSLNPRWDGERLYQEARKIVGAMVQIITYRDYLPLVLGPTAMRK (SEQ ID NO: 24). In some embodiments, an epitope comprises one or more additional sequences 5′ and/or 3′ to the epitope sequence.

In some embodiments, an epitope that is recognized by a MPO autoantibody comprises GYNAWRRFCGLPQPETVGQLGTVLRNLKLARKLMEQYGTPNNIDIWM GGVSEPLKRKGRVGPLLACIIGTQFRKLRDGDRFWWENEGVFSMQQRQALAQIS LPRIICDNTGITTVSKNNIFMSNSYPRDFVNCSTLPALNLASWREAS (SEQ ID NO. 25). In some embodiments, an epitope comprises one or more additional sequences 5′ and/or 3′ to one or more of any of the epitope sequence. In some embodiments, an epitope comprises one or more additional sequences 5′ and/or 3′ to the epitope sequence.

In some embodiments, an epitope that is recognized by a MPO autoantibody comprises RKIVGAMVQIITY (SEQ ID NO: 26). In some embodiments, an epitope comprises one or more additional sequences 5′ and/or 3′ to the epitope sequence.

In some embodiments, an epitope that is recognized by a MPO autoantibody comprises RKIVGAMVQIITYRD (SEQ ID NO. 27). In some embodiments, an epitope comprises one or more additional sequences 5′ and/or 3′ to the epitope sequence.

In some embodiments, an epitope that is recognized by a MPO autoantibody comprises WTPGVKRNGF (Amino Acids 213-222) (SEQ ID NO: 28). In some embodiments, an epitope comprises one or more additional sequences 5′ and/or 3′ to the epitope sequence.

In some embodiments, an epitope that is recognized by a MPO autoantibody comprises RLDNRYQPMEPN (AA 511-522) (SEQ ID NO: 29). In some embodiments, an epitope comprises one or more additional sequences 5′ and/or 3′ to the epitope sequence.

In some embodiments, an epitope that is recognized by a MPO autoantibody comprises SARIPCFLAG (aa 393-402) (SEQ ID NO: 30). In some embodiments, an epitope comprises one or more additional sequences 5′ and/or 3′ to the epitope sequence. In some embodiments, an epitope that is recognized by a MPO autoantibody comprises WDGERLYQEA (aa 437-446) (SEQ ID NO: 31). In some embodiments, an epitope comprises one or more additional sequences 5′ and/or 3′ to the epitope sequence.

In some embodiments, an epitope that is recognized by a MPO autoantibody comprises YRSYNDSVDP (aa 479-488) (SEQ ID NO: 32). In some embodiments, an epitope comprises one or more additional sequences 5′ and/or 3′ to the epitope sequence.

In some embodiments, an epitope that is recognized by a MPO autoantibody comprises FMSNSYPRD (aa 717-726) (SEQ ID NO: 33). In some embodiments, an epitope comprises one or more additional sequences 5′ and/or 3′ to the epitope sequence.

In some embodiments, an epitope that is recognized by a MPO autoantibody comprises GSASPMELLS (aa 91-100) (SEQ ID NO: 34). In some embodiments, an epitope comprises one or more additional sequences 5′ and/or 3′ to the epitope sequence.

In some embodiments, one or more polypeptides that specifically bind to an anti-MPO autoantibody comprises a variant of a MPO polypeptide. In some embodiments, a variant is an inactive variant as compared to a wild-type MPO polypeptide.

In some embodiments, a variant comprises a mutation at one or more or all of 261 (His), 316 (Cys), 405 (Arg) and 257 (Gln) of SEQ ID NO: 4.

In some embodiments, a variant comprises a mutation at 261 (His) of SEQ ID NO: 4. In some embodiments, a variant comprises a Histidine to Alanine mutation at position 261 of SEQ ID NO: 4. In some embodiments, a variant comprising a mutation at 261 (His) of SEQ ID NO: 4 further comprises one or more additional mutations at SEQ ID NO: 4, e.g., as described herein.

In some embodiments, a variant comprises a mutation at 405 (Arg) of SEQ ID NO: 4. In some embodiments, a variant comprising a mutation at 405 (Arg) of SEQ ID NO: 4 further comprises one or more additional mutations at SEQ ID NO: 4, e.g., as described herein.

In some embodiments, a variant comprises a mutation at 257 (Gln) of SEQ ID NO: 4. In some embodiments, a variant comprising a mutation at 257 (Gln) of SEQ ID NO: 4 further comprises one or more additional mutations at SEQ ID NO: 4, e.g., as described herein.

In some embodiments, a variant comprises a mutation at 316 (Cys) of SEQ ID NO: 4. In some embodiments, a variant comprises a Cysteine to Alanine mutation at position 316 of SEQ ID NO: 1. In some embodiments, a variant comprising a mutation at 316 (Cys) of SEQ ID NO: 4 further comprises one or more additional mutations at SEQ ID NO: 4, e.g., as described herein.

In some embodiments, a variant comprises a mutation at 261 (His), 405 (Arg), 316 (Cys) and 257 (Gln) of SEQ ID NO: 4. In some embodiments, a variant comprising a mutation at 261 (His) 405 (Arg), 316 (Cys) and 257 (Gln) of SEQ ID NO: 4 further comprises one or more additional mutations at SEQ ID NO: 4, e.g., as described herein.

In some embodiments, a variant comprises an MPO polypeptide or a fragment thereof having one or more mutations at a glycosylation site. In some embodiments, a mutation is at an Asparagine residue such that a glycosylation site is altered. In some embodiments, a mutation comprises a mutation at Asparagine 323, Asparagine 355, Asparagine 391, Asparagine 483, Asparagine 729, or combinations thereof.

In some embodiments, a glycoengineered polypeptide comprises an MPO polypeptide or a fragment thereof having one or more native glycosylation sites. In some embodiments, a glycoengineered polypeptide comprises an MPO polypeptide or a fragment thereof having 1, 2, 3, 4, 5, or 6 native glycosylation sites. In some embodiments, one or more native glycosylation sites comprise N139, N323, N355, N391, N483, and/or N729.

In some embodiments, a glycoengineered polypeptide comprises an MPO polypeptide or a fragment thereof having a native glycosylation site N139.

In some embodiments, a glycoengineered polypeptide comprises an MPO polypeptide or a fragment thereof having a native glycosylation site N323.

In some embodiments, a glycoengineered polypeptide comprises an MPO polypeptide or a fragment thereof having a native glycosylation site N355.

In some embodiments, a glycoengineered polypeptide comprises an MPO polypeptide or a fragment thereof having a native glycosylation site N391.

In some embodiments, a glycoengineered polypeptide comprises an MPO polypeptide or a fragment thereof having a native glycosylation site N483.

In some embodiments, a glycoengineered polypeptide comprises an MPO polypeptide or a fragment thereof having a native glycosylation site N729.

In some embodiments, a glycoengineered polypeptide comprises an MPO polypeptide or a fragment thereof having an engineered glycosylation site comprising the sequence GGGGANSTAPAPAPA (SEQ ID NO: 37).

In some embodiments, a glycoengineered polypeptide comprises a MPO polypeptide or a fragment thereof having one or more native glycosylation sites and one engineered glycosylation site, e.g., having the sequence GGGGANSTAPAPAPA (SEQ ID NO: 37).

In some embodiments, a glycoengineered polypeptide comprises an MPO polypeptide or a fragment thereof having six native glycosylation sites: N139, N323, N355, N391, N483, and N729 and one engineered glycosylation site with the sequence GGGGANSTAPAPAPA (SEQ ID NO: 37).

In some embodiments, a glycoengineered polypeptide further comprises one or more additional elements. In some embodiments, one or more additional elements comprise: (a) a linker, (b) a spacer, (c) a cleavage peptide, e.g., an IRES or a protease cleavage site, (d) a signal peptide, (e) a tag, e.g., a cleavable tag, (f) a half-life extender domain, e.g., an Fc domain or albumin, or (g) any combination of (a)-(f).

In some embodiments, a glycoengineered polypeptide comprises a tag, e.g., a His tag.

In some embodiments, a glycoengineered polypeptide comprises the following sequence (SEQ ID NO: 43):

MASRLVRVLAAAMLVAAAVSAAPAVLGEVDTSLVLSSMEEAKQLVD
KAYKERRESIKQRLRSGSASPMELLSYFKQPVAATRTAVRAADYLHVALDLLERKLRS
LWRRPFNVTDVLTPAQLNVLSKSSGCAYQDVGVTCPEQDKYRTITGMCNNRRSPTLG
ASNRAFVRWLPAEYEDGFSLPYGWTPGVKRNGFPVALARAVSNEIVRFPTDQLTPDQE
RSLMFMQWGQLLDADLDFTPEPAARASFVTGVNCETSCVQQPPCFPLKIPPNDPRIKNQ
ADCIPFFRSAPACPGSNITIRNQINALTSFVDASMVYGSEEPLARNLRNMSNQLGLLAVN
QRFQDNGRALLPFDNLHDDPCLLTNRSARIPCFLAGDTRSSEMPELTSMHTLLLREHNR
LATELKSLNPRWDGERLYQEARKIVGAMVQIITYRDYLPLVLGPTAMRKYLPTYRSYN
DSVDPRIANVFTNAFRYGHTLIQPFMFRLDNRYQPMEPNPRVPLSRVFFASWRVVLEG
GIDPILRGLMATPAKLNRQNQIAVDEIRERLFEQVMRIGLDLPALNMQRSRDHGLPGYN
AWRRFCGLPQPETVGQLGTVLRNLKLARKLMEQYGTPNNIDIWMGGVSEPLKRKGRV
GPLLACIIGTQFRKLRDGDRFWWENEGVFSMQQRQALAQISLPRIICDNTGITTVSKNNI
FMSNSYPRDFVNCSTLPALNLASWREASHHHHHHHHHH

In some embodiments, a glycoengineered polypeptide comprises a sequence with at least 85%, at least 90%, at least 95%, at least 99% identity to SEQ ID NO: 43. In some embodiments, a glycoengineered polypeptide comprises the sequence of SEQ ID NO: 43.

In some embodiments, one or more polypeptides that specifically bind to an anti-MPO autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 4.

In some embodiments, one or more polypeptides that specifically bind to an anti-MPO autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 43.

In some embodiments, one or more polypeptides that specifically bind to an anti-MPO autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 42.

In some embodiments, one or more polypeptides that specifically bind to an anti-MPO autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 46.

In some embodiments, one or more polypeptides that specifically bind to an anti-MPO autoantibody comprises a full length MPO protein, e.g., as provided in SEQ ID NO: 4 with or without the signal peptide.

In some embodiments, one or more polypeptides that specifically bind to an anti-MPO autoantibody comprise an MPO protein, e.g., as provided in SEQ ID NO: 46.

In some embodiments, a first moiety comprises 1, 2, 3, 4, 5, or more peptides that specifically bind to an anti-MPO autoantibody.

In some embodiments, one or more peptides of a first moiety that specifically bind to an anti-MPO autoantibody are the same, e.g., the one or more peptides have the same sequence of a MPO polypeptide, or a fragment or a variant thereof. In some embodiments, the one or more peptides having the same sequence of a MPO polypeptide, or a fragment or a variant thereof are separated by one or more intervening sequences (e.g., spacers and/or linkers). In some embodiments, the one or more peptides having the same sequence of a MPO polypeptide, or a fragment or a variant thereof are not separated by one or more intervening sequences (e.g., spacers and/or linkers).

In some embodiments, one or more peptides of a first moiety that specifically bind to an anti-MPO autoantibody are different, e.g., the one or more peptides do not have the same sequence of a MPO polypeptide, or a fragment or a variant thereof. In some embodiments, the one or more peptides having different sequences of a MPO polypeptide, or a fragment or a variant thereof are separated by one or more intervening sequences (e.g., spacers and/or linkers). In some embodiments, the one or more peptides different sequences of a MPO polypeptide, or a fragment or a variant thereof are not separated by one or more intervening sequences (e.g., spacers and/or linkers).

In some embodiments, each of the one or more peptides having different sequences of a MPO polypeptide, or a fragment or a variant thereof specifically bind to an anti-MPO autoantibody or a fragment thereof.

In some embodiments, a linker separating one or more peptides of a first moiety comprises a Gly-Ser linker, or an a EAAAK linker. In some embodiments, a linker comprises a (Gly-Gly-Gly-Gly-Ser) n linker, wherein n is an integer between 0 to 20.

In some embodiments, one or more peptides that specifically bind to an anti-MPO autoantibody are each conjugated to a second moiety.

In some embodiments, one or more peptides that specifically bind to an anti-MPO autoantibody are not each conjugated to the second moiety.

In some embodiments, one or more peptides that specifically bind to an anti-MPO autoantibody are conjugated to each other, e.g., are situated on one polypeptide.

In some embodiments, one or more peptides that specifically bind to an anti-MPO autoantibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an anti-MPO autoantibody are not separated by a protease cleavage site or an IRES, e.g., is expressed as a fusion protein.

In some embodiments, a first moiety comprises one or more peptides that specifically binds to an anti-MPO antibody. In some embodiments, one or more peptides that specifically bind to an anti-MPO antibody comprise an MPO polypeptide, or a variant or a fragment thereof. In some embodiments, a first moiety further comprises: (a) a linker, (b) a spacer, (c) a cleavage peptide, e.g., an IRES or, a protease cleavage site, (d) a signal peptide, (e) a tag, e.g., a cleavable tag, (f) a half-life extender domain, e.g., an Fc domain or albumin, (g) any combination of (a)-(f).

In some embodiments, one or more peptides that specifically bind to an anti-MPO antibody comprise an antibody agent. In some embodiments, the antibody agent comprises an antigen binding fragment. In some embodiments, the antibody agent comprises a full antibody, a Fab fragment, an scFv, a nanobody, a duobody, a single domain antibody (e.g., a VHH). In some embodiments, the antibody agent comprises a VHH, e.g., a camelid VHH or a bivalent VHH.

### Combination of PR3 and MPO Peptides

In some embodiments of a glycoengineered polypeptide disclosed herein, when a first moiety comprises one or more anti-PR3 autoantibody binding polypeptides, the first moiety further comprises one or more anti-MPO autoantibody binding polypeptides.

In some embodiments of a glycoengineered polypeptide disclosed herein, when a first moiety comprises one or more anti-MPO autoantibody binding polypeptides, the first moiety further comprises one or more anti-PR3 autoantibody binding polypeptides.

In some embodiments, a glycoengineered polypeptide disclosed herein comprises a first moiety comprising (i) one or more anti-PR3 autoantibody binding polypeptides and (ii) one or more anti-MPO autoantibody binding polypeptides.

In some embodiments, the one or more anti-PR3 autoantibody binding polypeptides and the one or more anti-MPO autoantibody binding polypeptides are situated on the same polypeptide.

In some embodiments, the one or more anti-PR3 autoantibody binding polypeptides and the one or more anti-MPO autoantibody binding polypeptides are situated on different polypeptides.

In some embodiments, the one or more anti-PR3 autoantibody binding polypeptides are situated on a first glycoengineered polypeptide and the one or more anti-MPO autoantibody binding polypeptides are situated on a second glycoengineered polypeptide.

In some embodiments, each of the anti-PR3 autoantibody binding polypeptides and each of the anti-MPO autoantibody binding polypeptides are conjugated to a second moiety.

### Second Moiety

A glycoengineered polypeptide disclosed herein comprises a first moiety that specifically binds to an anti-neutrophil autoantibody or a fragment or a complex thereof; and a second moiety comprising one or more glycans conjugated to the first moiety at one or more glycosylation sites.

Without being bound by any particular theory, glycan engagement with endocytic carbohydrate binding proteins and receptors enables different biological pathways. These essential biological pathways are involved in modulating immune responses, mediating protein clearance, protein turnover, and controlling trafficking of soluble glycoproteins, glycolipids and any natural molecule containing a glycan moiety. The glycan-receptor interaction is determined by the glycan structure. Glycan binding receptors are highly diverse and can be exploited by glycoengineering to develop novel therapeutics based on the concept of glycan-mediated protein degradation to treat different diseases, which include but are not limited to autoimmune disorders as disclosed herein.

Further without being bound by any particular theory a glycoengineered polypeptide comprising a second moiety having one or more glycans, as described herein, is expected to activate natural degradation pathways.

In some embodiments, a second moiety of a glycoengineered polypeptide disclosed herein comprises one or more glycans and specifically binds to one or more endocytic receptors. Endocytic receptors as described herein capture glycoproteins via specific glycan structures to mediate degradation, e.g., lysosomal degradation. Endocytic receptors are ubiquitous in human and can be found on different cells.

In some embodiments, an endocytic receptor is or comprises an endocytic lectin. In some embodiments, the endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, DNGR1, CLEC12B, DEC-205, CLEC10, and mannose 6 phosphate receptor (M6PR), or a combination thereof.

In some embodiments, provided herein is a glycoengineered polypeptide comprising a first moiety that specifically binds to a target protein (e.g., an anti-neutrophil autoantibody) and a second moiety comprising a glycan comprising terminal GlcNAc.

In some embodiments, provided herein is a glycoengineered polypeptide comprising a first moiety that specifically binds to a target protein (e.g., an anti-neutrophil autoantibody) and a second moiety comprising a glycan comprising terminal GalNAc.

In some embodiments, provided herein is a glycoengineered polypeptide comprising a first moiety that specifically binds to a target protein (e.g., an anti-neutrophil autoantibody) and a second moiety comprising a glycan comprising terminal Gal.

In some embodiments, a glycoengineered polypeptide provided herein can comprise (i) a binding specificity to one or more target protein(s) (e.g., one or more an anti-neutrophil autoantibodies) and (ii) one or more N-glycan(s) with binding specificities to one or more endocytic receptor(s).

In some embodiments, a glycoengineered polypeptide comprises one type of N-glycan with binding specificity to one type of endocytic receptor.

In some embodiments, a glycoengineered polypeptide comprises one or more N-glycosylation sites in a first moiety. In some embodiments, one or more N-glycosylation sites in a first moiety are native N-glycosylation sites. In some embodiments, one or more N-glycosylation sites in a first moiety are engineered N-glycosylation sites. In some embodiments, a glycoengineered polypeptide comprises one or more native N-glycosylation sites and one or more engineered N-glycosylation sites.

In some embodiments, a second moiety comprising one or more glycans is conjugated, e.g., linked, to a first moiety at one or more N-glycosylation sites.

In some embodiments, a glycoengineered polypeptide comprises 1, 2, 3, 4, 5, 6, 7, 8, 9, 10 or more N-glycosylation sites (or glycosites; such as an N-glycosylation consensus sequence). These N-glycosylation sites can be glycosylated by an N-glycan such that the resulting glycoengineered bifunctional binding protein can engage with or bind to 1, 2, 3, 4, 5, 6, 7, 8, 9, 10 or more endocytic receptor molecules.

In some embodiments, a glycoengineered polypeptide comprises two types of N-glycans with binding specificities to two different endocytic receptors. In certain embodiments, a glycoengineered polypeptide provided herein can comprise 1, 2, 3, 4, 5, 6, 7, 8, 9, 10 or more polypeptide chains. Each chain can be produced in a different cell line. In certain embodiments, the glycoengineered polypeptide can be an antibody and one type of N-glycan is on the Fc domain and another type of N-glycan is on the Fab domain (eg, the variable regions) of the antibody.

In some embodiments, a glycoengineered polypeptide comprises: (i) a first type of N-glycan with binding specificity to a first endocytic receptor wherein the first type of N-glycan is present at 1, 2, 3, 4, 5, 6, 7, 8, 9, 10 or more glycosites thus engaging with or binding to 1, 2, 3, 4, 5, 6, 7, 8, 9, 10 or more molecular of the first endocytic carbohydrate-binding protein or receptor; and (ii) a second type of N-glycan with binding specificity to a second endocytic receptor wherein the second N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the second endocytic receptor(s).

In some embodiments, a glycoengineered polypeptide comprises: (i) a first type of N-glycan with binding specificity to a first endocytic receptor wherein the first type of N-glycan is present at 1, 2, 3, 4, 5, 6, 7, 8, 9, 10 or more glycosites thus engaging with or binding to 1, 2, 3, 4, 5, 6, 7, 8, 9, 10 or more molecular of the first endocytic receptor; (ii) a second type of N-glycan with binding specificity to a second endocytic receptor wherein the second N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the second endocytic receptor(s); and (iii) a third type of N-glycan with binding specificity to a third endocytic receptor wherein the third N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the third endocytic receptor(s).

In some embodiments, a glycoengineered polypeptide provided herein has 1, 2, 3, 4, 5, 6, 7, 8, 9, 10 or more glycosites. In some embodiments, in a population of glycoengineered polypeptides, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycosites in the population at one specific position are glycosylated. In certain embodiments, in a population of glycoengineered polypeptides, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycosites in the population are glycosylated. N-glycans that can be present at the glycosites of the glycoengineered polypeptide provided herein are described herein.

In some embodiments, a glycosite is an N-glycosylation consensus sequence. The consensus sequence can be N—X—S/T, or N—X—C, wherein X is any amino acid except proline.

In some embodiments, a glycosite is or comprises the sequence of GGGGANSTAPAPAPA (SEQ ID NO: 37).

In some embodiments, an N-glycan is conjugated to the glycoengineered polypeptide at at least one, two, three, or four N-glycosylation sites.

In some embodiments, an N-glycan is conjugated to the glycoengineered polypeptide at one, two, three, or four N-glycosylation sites.

In some embodiments, an N-glycosylation site is naturally occurring.

In some embodiments, an N-glycosylation site is engineered into the amino acid sequence of the first moiety.

In certain embodiments, one or more of the N-glycosylation sites are engineered into the amino acid sequence of the first moiety of the glycoengineered polypeptide (i.e. one or more of the N-glycosylation sites are not present in a wild-type, or naturally occurring form of the first moiety). In certain embodiments, at least one of the N-glycosylation sites is engineered into the amino acid sequence of the first moiety of the glycoengineered polypeptide. In certain embodiments, at least two of the N-glycosylation sites are engineered into the amino acid sequence of the first moiety of the glycoengineered polypeptide. In certain embodiments, at least three of the N-glycosylation sites are engineered into amino acid sequence of the first moiety of the glycoengineered polypeptide. In certain embodiments, at least four of the N-glycosylation sites are engineered into the amino acid sequence of the first moiety of the glycoengineered polypeptide. In certain embodiments, one or more of the engineered N-glycosylation sites are glycotags fused to the N- and/or C-terminus of the amino acid sequence of the first moiety of the glycoengineered polypeptide via a peptide linker. In certain embodiments, a glycotag is fused to the N-terminus of first moiety of the glycoengineered polypeptide. In certain embodiments, a glycotag is fused to the C-terminus of first moiety of the glycoengineered polypeptide. In certain embodiments, a glycotag is fused to the N- and the C-terminus of first moiety of the glycoengineered polypeptide. In certain embodiments, one or more of the N-glycosylation sites are natural N-glycosylation sites (i.e. one or more of the N-glycosylation sites are present in a wild-type, or naturally occurring form of the first moiety). In certain embodiments, at least one of the N-glycosylation sites is a natural N-glycosylation site. In certain embodiments, at least two of the N-glycosylation sites are natural N-glycosylation sites.

In some embodiments, provided herein is a glycoengineered polypeptide that specifically binds to a target protein associated with a disease (e.g., an anti-neutrophil autoantibody), comprising a first moiety and a second moiety. In some embodiments, provided herein is a glycoengineered polypeptide comprising a first moiety that specifically binds to a target protein associated with a disease (e.g., an anti-neutrophil autoantibody), and a second moiety that binds specifically to an endocytic receptor, wherein the second moiety comprises a glycan structure.

In some embodiments, provided herein is a glycoengineered polypeptide comprising a first moiety that specifically binds to a target protein and a second moiety comprising an N-glycan selected from the group consisting of GlcNAc2Man3GlcNAc2, GalNAc2GlcNAc2Man3 GlcNAc2, Gal2GlcNAc2Man3GlcNAc2, Man3 GlcNAc, GlcNAc 1Man3 GlcNAc2, Gal2GlcNAc2Man3 GlcNAc2, Gal 1 GlcNAc2Man3 GlcNAc2, GalNAc 1 GlcNAc2Man3 GlcNAc2, GlcNAc3Man3 GlcNAc2, GlcNAc4Man3 GlcNAc2, Gal3GlcNAc3Man3 GlcNAc2, GalNAc3 GlcNAc3Man3 GlcNAc2, GalNAc4GlcNAc4Man3GlcNAc2, Gal4GlcNAc4Man3GlcNAc2, or Man-6-P—N-glycan.

In some embodiments, increasing the number of glycan structures on a glycoengineered polypeptide increases the rate of lysosomal degradation as compared to an otherwise similar glycoengineered polypeptide with fewer glycan structures.

In some embodiments, the number of glycan structures on a glycoengineered polypeptide disclosed herein is 1 or more, 2 or more, 3 or more, 4 or more, 5 or more, 6 or more, 7 or more, 8 or more, 9 or more or 10 or more glycan structures.

In some embodiments, a glycoengineered polypeptide disclosed herein comprises a glycan structure having a monoantennary structure.

In some embodiments, a glycoengineered polypeptide disclosed herein comprises a glycan structure having a biantennary structure.

In some embodiments, a glycoengineered polypeptide disclosed herein comprises a glycan structure having a triantennary structure.

In some embodiments, a glycoengineered polypeptide disclosed herein comprises a glycan structure having a tetraantennary structure.

In some embodiments, the glycan structure comprises a biantennary structure. In some embodiments, the glycan structure comprises a biantennary GalNAc. In some embodiments, the biantennary GalNac binds to an asialoglycoprotein receptor (ASGPR) or a fragment or variant thereof, or a complex comprising ASGPR.

In some embodiments, the N-glycan has a structure of:

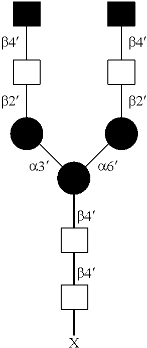

wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.

In some embodiments, the N-glycan specifically binds to one or more endocytic receptors, e.g., that mediate lysosomal degradation. In some embodiments, the N-glycan specifically binds to ASGPR.

In some embodiments, the endocytic receptor is or comprises ASGPR or a fragment or variant thereof, or a complex comprising ASGPR. In some embodiments, when the endocytic receptor is ASGPR, the glycan structure of the second moiety comprises a terminal GalNac.

ASGPR-mediated degradation in the hepatocyte has many applications. ASPGR binding to the N-glycan structure disclosed herein can result in the selective degradation of one or more target proteins (e.g., an anti-neutrophil autoantibody). By way of example, ASGPR-mediated degradation can lead to removal of cytokines, chemokines and hormones. Additionally, ASGPR-mediated degradation can be used for the delivery of the target molecules to the hepatocyte endosome. Thus, ASGPR-mediated degradation is applicable for various diseases, while limiting systemic toxicity.

In certain embodiments, the 1, 2, 3, 4, 5, 6, 7, 8, 9, 10 or more N-glycosylation sites can be glycosylated by the N-glycan such that the resulting glycoengineered polypeptide can engage with or bind to 1, 2, 3, 4, 5, 6, 7, 8, 9, 10 or more endocytic receptor molecules. In certain embodiments, the glycoengineered polypeptide is glycosylated by the N-glycan at at least 2, at least 3, at least 4, at least 5, at least 6, at least 7, at least 8, at least 9, or at least 10 of the N-glycosylation sites. In certain embodiments, the glycoengineered polypeptide is glycosylated by the N-glycan at at least 2 N-glycosylation sites. In certain embodiments, the glycoengineered polypeptide is glycosylated by the N-glycan at at least 3 N-glycosylation sites. In certain embodiments, the glycoengineered polypeptide is glycosylated by the N-glycan at at least 4 N-glycosylation sites. In certain embodiments, the glycoengineered polypeptide is glycosylated by the N-glycan at at least 5 N-glycosylation sites. In certain embodiments, the glycoengineered polypeptide is glycosylated by the N-glycan at at least 6 N-glycosylation sites. In certain embodiments, the glycoengineered polypeptide is glycosylated by the N-glycan at at least 7 N-glycosylation sites. In certain embodiments, the glycoengineered polypeptide is glycosylated by the N-glycan at at least 8 N-glycosylation sites. In certain embodiments, the glycoengineered polypeptide is glycosylated by the N-glycan at at least 9 N-glycosylation sites. In certain embodiments, the glycoengineered polypeptide is glycosylated by the N-glycan at at least 10 N-glycosylation sites.

In certain embodiments, the glycoengineered polypeptide is glycosylated by the N-glycan at 2 N-glycosylation sites. In certain embodiments, the glycoengineered polypeptide is glycosylated by the N-glycan at 3 N-glycosylation sites. In certain embodiments, the glycoengineered polypeptide is glycosylated by the N-glycan at 4 N-glycosylation sites. In certain embodiments, the glycoengineered polypeptide is glycosylated by the N-glycan at 5 N-glycosylation sites. In certain embodiments, the glycoengineered polypeptide is glycosylated by the N-glycan at 6 N-glycosylation sites. In certain embodiments, the glycoengineered polypeptide is glycosylated by the N-glycan at 7 N-glycosylation sites. In certain embodiments, the glycoengineered polypeptide is glycosylated by the N-glycan at 8 N-glycosylation sites. In certain embodiments, the glycoengineered polypeptide is glycosylated by the N-glycan at 9 N-glycosylation sites. In certain embodiments, the glycoengineered polypeptide is glycosylated by the N-glycan at 10 N-glycosylation sites. In certain embodiments, the glycoengineered polypeptide is glycosylated at an Asn amino acid residue of the glycoengineered polypeptide. In certain embodiments, the N-glycosylation site is an N-glycosylation consensus sequence. In certain embodiments, the N-glycosylation site comprises a consensus sequence of N—X—S/T or N—X—C, wherein X is any amino acid except proline.

In certain embodiments, at least 10%, at least 20%, at least 30%, at least 40%, at least 50%, at least 60%, at least 70%, at least 80%, at least 90%, at least 95, or at least 98% of the N-glycosylation sites are occupied by an N-glycan. In certain embodiments, at least 10%, at least 20%, at least 30%, at least 40%, at least 50%, at least 60%, at least 70%, at least 80%, at least 90%, at least 95, or at least 98% of the N-glycosylation sites are occupied by an N-glycan of the structure:

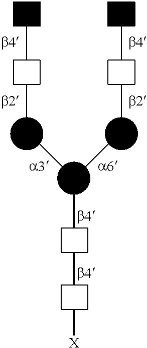

linked to the glycoengineered polypeptide at one or more N-glycosylation sites, wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide. In certain embodiments, at least 10% of the N-glycosylation sites are occupied by the N-glycan. In certain embodiments, at least 20% of the N-glycosylation sites are occupied by the N-glycan. In certain embodiments, at least 30% of the N-glycosylation sites are occupied by the N-glycan. In certain embodiments, at least 40% of the N-glycosylation sites are occupied by the N-glycan. In certain embodiments, at least 50% of the N-glycosylation sites are occupied by the N-glycan. In certain embodiments, at least 60% of the N-glycosylation sites are occupied by the N-glycan. In certain embodiments, at least 70% of the N-glycosylation sites are occupied by the N-glycan. In certain embodiments, at least 80% of the N-glycosylation sites are occupied by the N-glycan. In certain embodiments, at least 90% of the N-glycosylation sites are occupied by the N-glycan. In certain embodiments, at least 95% of the N-glycosylation sites are occupied by the N-glycan. In certain embodiments, at least 98% of the N-glycosylation sites are occupied by the N-glycan.

In certain embodiments, the N-glycan is linked to the glycoengineered polypeptide at at least one N-glycosylation site. In certain embodiments, the N-glycan is linked to the glycoengineered polypeptide at at least two N-glycosylation sites. In certain embodiments, the N-glycan is linked to the glycoengineered polypeptide at one, two, three, or four N-glycosylation sites. In certain embodiments, the N-glycan is linked to the glycoengineered polypeptide at one N-glycosylation site. In certain embodiments, the N-glycan is linked to the glycoengineered polypeptide at two N-glycosylation sites. In certain embodiments, the N-glycan is linked to the glycoengineered polypeptide at three N-glycosylation sites. In certain embodiments, the N-glycan is linked to the glycoengineered polypeptide at four N-glycosylation sites. In certain embodiments, the N-glycan is linked to the glycoengineered polypeptide at an Asn amino acid residue of the glycoengineered polypeptide. In certain embodiments, the N-glycan is linked to the glycoengineered polypeptide at an N-glycosylation consensus sequence. In certain embodiments, the N-glycan is linked to the glycoengineered polypeptide at a consensus sequence of N—X—S/T or N—X—C, wherein X is any amino acid except proline.

In certain embodiments, the glycoengineered polypeptide comprises two different N-glycans (i.e. a first and a second N-glycan), wherein each N-glycan is independently linked to the glycoengineered polypeptide at 1, 2, 3, 4, 5, 6, 7, 8, 9, 10 or more N-glycosylation sites, and wherein one of the N-glycans (i.e. the first N-glycan) has the structure:

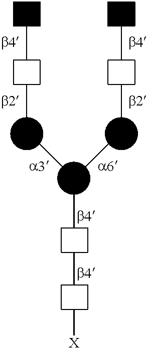

wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide. In certain embodiments, the different N-glycans specifically bind to different endocytic receptors. In certain embodiments, the first N-glycan specifically binds to ASGPR. In certain embodiments, the other N-glycan is an N-glycan described in PCT/EP2022/057556, which is incorporated herein by reference in its entirety. In certain embodiments, the first N-glycan is larger than the second N-glycan. In other embodiments, the first N-glycan is smaller than the second N-glycan. In certain embodiments, the N-glycosylation sites predominantly or exclusively occupied by the larger N-glycan are more sterically accessible than the N-glycosylation sites predominantly or exclusively occupied by the smaller N-glycan. In certain embodiments, the other N-glycan is A2. In certain embodiments, the other N-glycan is A1GalNAc1 or A2GalNAc1. In certain embodiments, the N-glycans are linked to the glycoengineered polypeptide at an Asn amino acid residue of the glycoengineered polypeptide. In certain embodiments, the N-glycans are linked to the glycoengineered polypeptide at an N-glycosylation consensus sequence. In certain embodiments, the N-glycans are linked to the glycoengineered polypeptide at a consensus sequence of N—X—S/T or N—X—C, wherein X is any amino acid except proline. In certain embodiments, the first N-glycan is linked to the glycoengineered polypeptide at 1, 2, 3, 4, 5, 6, 7, 8, 9, 10 or more N-glycosylation sites, and the second N-glycan is linked to the glycoengineered polypeptide at 1, 2, 3, 4, 5, 6, 7, 8, 9, 10 or more N-glycosylation sites.

In certain embodiments, the glycoengineered polypeptide further comprises a third N-glycan, wherein the third N-glycan is linked to the glycoengineered polypeptide at 1, 2, 3, 4, 5, 6, 7, 8, 9, 10 or more N-glycosylation sites. In certain embodiments, the third N-glycan specifically binds to a different endocytic receptor than the first and/or second N-glycan. In certain embodiments, the third N-glycan is an N-glycan described in PCT/EP2022/057556, which is incorporated herein by reference in its entirety. In certain embodiments, the third N-glycan is A2. In certain embodiments, the third N-glycan is A1GalNAc1 or A2GalNAc1. In certain embodiments, the third N-glycan is linked to the glycoengineered polypeptide at an Asn amino acid residue of the glycoengineered polypeptide. In certain embodiments, the third N-glycan is linked to the glycoengineered polypeptide at an N-glycosylation consensus sequence. In certain embodiments, the third N-glycan is linked to the glycoengineered polypeptide at a consensus sequence of N—X—S/T or N—X—C, wherein X is any amino acid except proline.

In certain embodiments, the second and/or third N-glycan specifically bind to an endocytic lectin. In some embodiments, the endocytic lectin is a mannose binding receptor. In some embodiments, the endocytic lectin is a Cluster of Differentiation 206 (CD206) receptor. In some embodiments, the endocytic lectin is a DC-SIGN (Cluster of Differentiation 209 or CD209) receptor. In some embodiments, the endocytic lectin is a C-Type Lectin Domain Family 4 Member G (LSECTin) receptor. In some embodiments, the endocytic lectin is a macrophage inducible Ca2+-dependent lectin receptor (Mincle). In some embodiments, the endocytic receptor is L-SIGN CD209L. In some embodiments, the endocytic receptor is asialoglycoprotein (ASGPR). In some embodiments, the endocytic receptor is dectin-1. In some embodiments, the endocytic receptor is dectin-2. In some embodiments, the endocytic receptor is langerin. In some embodiments, the second and/or third N-glycan specifically bind to a receptor selected from the group consisting of macrophage mannose 2 receptor, BDCA-2, DCIR, MBL, MDL, MICL, CLEC2, CLEC10, DNGR1, CLEC12B, DEC-205, and mannose 6 phosphate receptor (M6PR).

CD206 is a C-type lectin and phagocytic/endocytic recycling and signaling receptor. CD206 is expressed primarily by M2 anti-inflammatory macrophages, dendritic cells, and live sinusoidal endothelial cells. DC-SIGN is a non-recycling, signaling receptor that targets both the ligand and receptor to the lysosome for degradation. LSECTin is expressed on liver sinusoidal endothelial cells.

In certain embodiments, the glycoengineered polypeptide is glycosylated at two or more N-glycosylation sites by an N-glycan of the structure:

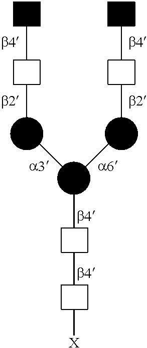

wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide, and wherein two of the N-glycosylation sites are separated by at least 5, at least 10, at least 20, at least 30, at least 40, at least 50, at least 60, at least 70, at least 80, at least 90, or at least 100 amino acids. In certain embodiments, the N-glycan is linked to the glycoengineered polypeptide at two N-glycosylation sites separated by a distance of about 5-10, about 10-20, about 20-30, about 30-40, about 40-50, about 50-60, about 60-70, about 70-80, about 80-90, about 90-100, about 100-150, about 150-200, or about 200-300 amino acids. In certain embodiments, the amino acid separation between the N-glycosylation sites is the number of amino acids between the terminal amino acids of the N-glycosylation consensus sequence. Without being bound by theory, the glycoengineered polypeptide folds in space and, thus, has a three-dimensional geometry in addition to its primary amino acid structure. Also without being bound by theory, this three-dimensional geometry, including the position of the N-glycan is not static but dynamic (see, for example, Re, S., et al Biophysical Reviews, 4, 179-187 (2012)). Notwithstanding, in certain embodiments, the distance between N-glycosylation sites and/or N-glycans on a glycoengineered polypeptide may be from an equilibrium geometry of the glycoengineered polypeptide, as determined by any standard means known in the art, including for example computational modelling studies. In certain embodiments, the N-glycan is linked to the glycoengineered polypeptide at two N-glycosylation sites separated by a distance of at least 1.0 nm. In certain embodiments, the N-glycan is linked to the glycoengineered polypeptide at two N-glycosylation sites separated by a distance of about 1.0-5.0 nm. In certain embodiments, the N-glycan is linked to the glycoengineered polypeptide at two N-glycosylation sites separated by a distance of about 1.5-3.0 nm. In certain embodiments, the N-glycan is linked to the glycoengineered polypeptide at two N-glycosylation sites separated by a distance of about 1.5-2.5 nm. In certain embodiments, the N-glycan is linked to the glycoengineered polypeptide at three N-glycosylation sites each separated by a distance of about 1.0-5.0 nm. In certain embodiments, the N-glycan is linked to the glycoengineered polypeptide at three N-glycosylation sites each separated by a distance of about 1.5-3.0 nm. In certain embodiments, the N-glycan is linked to the glycoengineered polypeptide at three N-glycosylation sites each separated by a distance of about 1.5-2.5 nm. In certain embodiments, the N-glycans are separated by a distance of at least 1.0 nm. In certain embodiments, the N-glycans are separated by a distance of about 1.0 to about 5.0 nm. In certain embodiments, the N-glycans are separated by a distance of about 1.5 to about 2.5 nm. In certain embodiments, the distance between the N-glycosylation sites and/or N-glycans is chosen to minimize steric hindrance, for example between the glycoengineered polypeptide(s), the target protein(s), and/or the ASGPR receptor(s). In certain embodiments, the distance between the N-glycosylation sites and/or N-glycans is chosen based on the separation of ASGPR receptors on a cell surface. In certain embodiments, the distance between the N-glycosylation sites and/or N-glycans is chosen to be similar (e.g. no more than twice, or no less than half) to the separation of ASGPR receptors on a cell surface. In certain embodiments, the N-glycan is linked to the glycoengineered polypeptide at an Asn amino acid residue of the glycoengineered polypeptide. In certain embodiments, the N-glycan is linked to the glycoengineered polypeptide at an N-glycosylation consensus sequence. In certain embodiments, the N-glycan is linked to the glycoengineered polypeptide at a consensus sequence of N—X—S/T or N—X—C, wherein X is any amino acid except proline.

### Nucleic Acid Sequences Encoding Glycoengineered Polypeptides

The present disclosure, among other things, provides nucleic acid sequences encoding glycoengineered polypeptides as described herein.

In some embodiments, a nucleic acid sequence is or comprises single stranded DNA (e.g., as in certain viral vectors). In some embodiments, a nucleic acid is or comprises double stranded DNA (e.g., as in certain viral vectors and/or certain plasmids). In some embodiments, a nucleic acid is or comprises RNA (e.g., as in certain viral vectors and/or as in mRNA therapeutics), etc.

Nucleic acids encoding glycoengineered polypeptides may be modified to include codons that are optimized for expression in a particular cell type (e.g., a Leishmania cell) or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acids encoding glycoengineered polypeptides described herein, in whole or in part, may include an altered sequence to optimize codon usage for a particular cell type (e.g., a eukaryotic or prokaryotic cell). For example, a coding sequence for an antibody agent (e.g., antigen binding fragment) as described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host (e.g., Leishmania host cell). Selection of vectors and methods to construct them are commonly known to persons of ordinary skill in the art.

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding a glycoengineered polypeptide described herein under control of transcriptional and/or translational control signals. These methods may include in vitro recombinant DNA and synthetic techniques and in vivo recombination (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

### Compositions and Pharmaceutical Compositions

A composition disclosed herein may comprise and/or deliver one or more glycoengineered polypeptides disclosed herein or nucleic acids encoding one or more glycoengineered polypeptides disclosed herein.

In some embodiments, a composition disclosed herein comprises a glycoengineered polypeptide comprising a first moiety and a second moiety. In some embodiments, a composition disclosed herein comprises a plurality of glycoengineered polypeptides comprising a first moiety and a second moiety.

In some embodiments, a composition comprising a plurality of glycoengineered polypeptides comprises 1, 2, 3, 4, 5, or more glycoengineered polypeptides comprising a first moiety that binds to an anti-neutrophil autoantibody (e.g., a first moiety that binds to the same anti-neutrophil autoantibody).

In some embodiments, a composition comprising a plurality of glycoengineered polypeptides comprises glycoengineered polypeptides having a first moiety that binds to an anti-PR3 autoantibody or a fragment thereof. In some embodiments, the glycoengineered polypeptides in the plurality each comprise the same first moiety. In some embodiments, the glycoengineered polypeptides in the plurality each comprise a different first moiety (e.g., a first glycoengineered polypeptide comprises a first moiety that binds to an anti-PR3 autoantibody or a fragment thereof, a second glycoengineered polypeptide comprises a different first moiety that binds to an anti-PR3 autoantibody or a fragment thereof, etc.).

In some embodiments, a composition comprising a plurality of glycoengineered polypeptides comprises glycoengineered polypeptides having a first moiety that binds to an anti-MPO autoantibody or a fragment thereof. In some embodiments, the glycoengineered polypeptides in the plurality each comprise the same first moiety. In some embodiments, the glycoengineered polypeptides in the plurality each comprise a different first moiety (e.g., a first glycoengineered polypeptide comprises a first moiety that binds to an anti-MPO autoantibody or a fragment thereof, a second glycoengineered polypeptide comprises a different first moiety that binds to an anti-MPO autoantibody or a fragment thereof, etc.).

In some embodiments, a composition comprising a plurality of glycoengineered polypeptides comprises 1, 2, 3, 4, 5, or more glycoengineered polypeptides each comprising a first moiety that binds to a different anti-neutrophil autoantibody (e.g., an anti-PR3 autoantibody or a fragment thereof, or an anti-MPO autoantibody or a fragment thereof. In some embodiments, a composition comprising a plurality of glycoengineered polypeptides comprises a first glycoengineered polypeptide comprising a first moiety that binds to an anti-neutrophil autoantibody (e.g., an anti-PR3 autoantibody or a fragment thereof) and a second glycoengineered polypeptide comprising a first moiety that binds to a different anti-neutrophil autoantibody (e.g., an anti-MPO autoantibody or a fragment thereof).

In some embodiments, a glycoengineered polypeptide disclosed herein (i) a first glycoengineered polypeptide comprising a first moiety that specifically binds to an anti-PR3 autoantibody or a fragment or a complex thereof, and (ii) a second glycoengineered polypeptide comprising a first moiety that specifically binds to an anti-MPO autoantibody or a fragment or a complex thereof.

In some embodiments, a glycoengineered polypeptide disclosed herein (i) a first glycoengineered polypeptide comprising a first moiety that specifically binds to an anti-PR3 autoantibody or a fragment or a complex thereof, and (ii) a second glycoengineered polypeptide comprising a first moiety that specifically binds to an anti-neutrophil antibody other than an anti-PR3 autoantibody, or a fragment or a complex thereof.

In some embodiments, a glycoengineered polypeptide disclosed herein (i) a first glycoengineered polypeptide comprising a first moiety that specifically binds to an anti-MPO autoantibody or a fragment or a complex thereof, and (ii) a second glycoengineered polypeptide comprising a first moiety that specifically binds to an anti-neutrophil antibody other than an anti-MPO autoantibody, or a fragment or a complex thereof.

In some embodiments, ratio of the first glycoengineered polypeptide to the second glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

In some embodiments, the ratio of the first glycoengineered polypeptide to the second glycoengineered polypeptide is about 1:5 to about 5:1; about 1:2.5 to about 2.5:1.

In some embodiments, the ratio of the first glycoengineered polypeptide to the second glycoengineered polypeptide is about 1:1.5 to about 1.5:1.

In some embodiments, the first glycoengineered polypeptide is present at an amount of about 10-90% and the second glycoengineered polypeptide is present at an amount of about 90-10%.

In some embodiments, the first glycoengineered polypeptide is present at an amount of about 20-80% and the second glycoengineered polypeptide is present at an amount of about 80-20%.

In some embodiments, the first glycoengineered polypeptide is present at an amount of about 30-70% and the second glycoengineered polypeptide is present at an amount of about 70-30%.

In some embodiments, the first glycoengineered polypeptide is present at an amount of about 40-60% and the second glycoengineered polypeptide is present at an amount of about 60-40%.

In some embodiments, the first glycoengineered polypeptide is present at an amount of about 10% and the second glycoengineered polypeptide is present at an amount of about 90%.

In some embodiments, the first glycoengineered polypeptide is present at an amount of about 20% and the second glycoengineered polypeptide is present at an amount of about 80%.

In some embodiments, the first glycoengineered polypeptide is present at an amount of about 30% and the second glycoengineered polypeptide is present at an amount of about 70%.

In some embodiments, the first glycoengineered polypeptide is present at an amount of about 40% and the second glycoengineered polypeptide is present at an amount of about 60%.

In some embodiments, the first glycoengineered polypeptide is present at an amount of about 50% and the second glycoengineered polypeptide is present at an amount of about 50%.

In some embodiments, the first glycoengineered polypeptide is present at an amount of about 60% and the second glycoengineered polypeptide is present at an amount of about 40%.

In some embodiments, the first glycoengineered polypeptide is present at an amount of about 70% and the second glycoengineered polypeptide is present at an amount of about 30%.

In some embodiments, the first glycoengineered polypeptide is present at an amount of about 80% and the second glycoengineered polypeptide is present at an amount of about 20%.

In some embodiments, the first glycoengineered polypeptide is present at an amount of about 90% and the second glycoengineered polypeptide is present at an amount of about 10%.

In some embodiments, disclosed herein is a composition comprising a population of glycoengineered polypeptides, wherein the population of glycoengineered polypeptides has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s).

In some embodiments, the homogeneity of the N-glycan profile at one or more of the N-glycosylation sites is determined by N-glycan analysis, glycopeptide analysis or intact protein analysis.

In some embodiments, the N-glycan profile comprises about 30% to 40%, about 40% to about 50%, about 50% to about 60%, about 60% to about 70%, about 70% to about 80%, about 80% to about 90%, or about 90% to about 100% of the N-glycan of the structure provided herein.

In some embodiments, the population of glycoengineered polypeptides has an N-glycan profile comprising about 30% to about 40%, about 40% to about 50%, about 50% to about 60%, about 60% to about 70%, about 70% to about 80%, about 80% to about 90%, or about 90% to about 100% of the N-glycan of the structure provided herein among all glycans in the N-glycan profile.

In some embodiments, a glycoengineered polypeptide disclosed herein or a composition comprising the same may be useful to treat and/or prevent a disease described herein (e.g., a disease associated anti-neutrophil autoantibodies), or to ameliorate a symptom associated with a disease, disorder or condition described herein.

The present disclosure also provides pharmaceutical compositions that, when administered to a subject (e.g., a human subject), e.g., when administered to a subject suffering from a disease associated with anti-neutrophil autoantibodies, deliver a glycoengineered polypeptide as described herein to such subject. Thus, in some embodiments, the present disclosure provides pharmaceutical compositions that comprise or deliver one or more glycoengineered polypeptides or one or more polynucleotides encoding such as described herein.

In some embodiments, a pharmaceutical composition is or comprises a composition according to the present disclosure.

Typically, a pharmaceutical composition includes a glycoengineered polypeptide as described herein, or a nucleic acid that encodes it in combination with one or more pharmaceutically acceptable carriers or excipients such as, for example one or more buffers, diluents, fillers, salts, solubilizers, stabilizers, and/or other materials as is known in the art. Those skilled in the art will be aware of a variety of carrier components appropriate to a particular active type (e.g., polypeptide versus nucleic acid, viral vector vs plasmid versus RNA, etc.) and/or route of administration (e.g., parenteral, enteral, etc.).

In some embodiments, a pharmaceutical composition, may comprise or deliver two or more different glycoengineered polypeptide, so that such agents may be administered in combination (e.g., substantially simultaneously or sequentially) to subject(s).

In some embodiments, a pharmaceutical composition may contain one or more agents that, for example, may improve stability of the composition and/or its active agent (e.g., to particular storage conditions and/or period(s) of time), facilitate delivery of the composition and/or its active agent, and/or otherwise enhance effectiveness (and/or reduce one or more undesirable side effects) of the active agent or composition once administered.

Alternatively or additionally, in some embodiments, a provided pharmaceutical composition may comprise or deliver another active agent in addition to an glycoengineered polypeptide as described herein.

Methods of Treatment and/or Prevention

The present disclosure, among other things, provides methods of treating and/or preventing an anti-neutrophil autoantibody associated disease in a subject comprising administering a composition as described herein, thereby improving at least one sign or symptom of the anti-neutrophil autoantibody associated disease in the subject after administration.

In some embodiments, provided herein are methods of treating and/or preventing a disease associated with anti-neutrophil autoantibodies, comprising administering a composition as described herein.

In some embodiments, a disease associated with anti-neutrophil autoantibodies is an autoimmune disease and/or an inflammatory disorder. In some embodiments, an autoimmune disease is ANCA vasculitis.

Among other things, disclosed herein is the identification of glycoengineered polypeptides that specially bind to anti-neutrophil autoantibodies or fragments or complexes thereof (e.g., anti-PR3 autoantibodies and/or anti-MPO autoantibodies). In some embodiments, glycoengineered polypeptides disclosed herein have therapeutic value, e.g., in the treatment of a disease associated with anti-neutrophil autoantibodies or fragments or complexes thereof (e.g., ANCA).

A glycoengineered polypeptide that specifically binds to an anti-neutrophil autoantibody (e.g., anti-PR3 autoantibody and/or anti-MPO autoantibody) of the present disclosure can be used, inter alia, to treat, prevent, and/or improve anti-neutrophil autoantibody associated diseases, including but not limited to any number of diseases in which the anti-neutrophil autoantibody levels are aberrantly high and/or in which a reduction of anti-neutrophil autoantibody levels is sought.

A subject to be treated with methods described herein can be e.g., a patient having, or at risk of having, or is diagnosed as having a disease associated with anti-neutrophil autoantibodies (e.g., ANCA vasculitis).

Those skilled in the art, reading the present disclosure, will appreciate that provided compositions, may be useful for treating a disease associated with anti-neutrophil autoantibodies. In some embodiments, a disease associated with anti-neutrophil autoantibodies has or is characterized as having increased levels of anti-neutrophil autoantibodies. In some embodiments, a disease associated with anti-neutrophil autoantibodies has or is characterized as having aberrant anti-neutrophil autoantibodies.

In some embodiments, a method of treating and/or preventing Granulomatosis with Polyangiitis (GPA)/cytoplasmic ANCA (Formerly called Wegener's Granulomatosis) in a subject comprises administering to a subject a composition according to the present disclosure. In some embodiment, a subject has an anti-PR3 autoantibody. In some embodiments, administration of a composition reduces a level of PR3 autoantibody as compared to a subject who has not been administered the composition or as compared to the same subject prior to administration of the composition. In some embodiments, a reduction in the level of the anti-PR3 autoantibody prevents neutrophil activation.

In some embodiments, a method of treating and/or preventing Microscopic Polyangiitis (MPA)/perinuclear ANCA in a subject comprises administering to a subject a composition according to the present disclosure. In some embodiment, a subject has a MPO autoantibody. In some embodiments, administration of a composition reduces a level of MPO autoantibody as compared to a subject who has not been administered the composition or as compared to the same subject prior to administration of the composition. In some embodiments, a reduction in the level of the MPO autoantibody prevents neutrophil activation.

In some embodiments, a composition according to the present invention is used to reduce and/or degrade anti-neutrophil autoantibodies (e.g., anti-PR3 autoantibodies and/or anti-MPO autoantibodies).

In some embodiments, a composition according to the present invention is used to reduce the risk of a disease associated with anti-neutrophil autoantibodies.

In some embodiments, a composition according to the present invention is used to ameliorate one or more symptoms of a disease associated with anti-neutrophil autoantibodies.

Also disclosed herein is a method comprising, assessing a level of an anti-neutrophil autoantibody in a sample from a subject, and administering a pharmaceutical composition disclosed herein, if the level of the anti-neutrophil autoantibody is higher than a comparator. In some embodiments, a comparator comprises a predetermined reference sample such as a sample obtained from an otherwise similar subject who does not have a disease or disorder, or a symptom of a disease or disorder.

In some embodiments, an anti-neutrophil autoantibody comprises: an anti-PR3 autoantibody or a fragment or a complex thereof; or an anti-MPO autoantibody or a fragment or a complex thereof, or a combination thereof.

### Administration

In some embodiments, of the disclosure, provided are methods comprising administering to a subject a composition according to the present disclosure. In some embodiments, a method comprises administering to a subject a pharmaceutical composition comprising a glycoengineered polypeptide according to the present disclosure.

Delivery of a glycoengineered polypeptide can be achieved e.g., by administration of a pharmaceutical composition as described herein, such as a pharmaceutical composition that comprises a glycoengineered polypeptide or a nucleic acid that encodes it, for example via oral ingestion, inhalation, topical application or parenteral administration (e.g., cutaneous, subcutaneous, intraperitoneal, intramuscular or intravenous injection). In some embodiments, administration is by intravenous or intramuscular injection. In some embodiments, local administration may be or comprise topical administration (e.g., to the skin) or parenteral administration (e.g., by injection to a site of deposition such as to the kidney).

In some embodiments, delivery of a glycoengineered polypeptide can be achieved e.g., by administration of a pharmaceutical composition as described herein, such as a pharmaceutical composition that comprises a glycoengineered polypeptide or a nucleic acid that encodes it, may be oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural). Those skilled in the art will be aware of typical guiding principles for formulation of pharmaceutical compositions for administration by such routes. For example, such techniques may include the step of bringing into association a glycoengineered polypeptide or a nucleic acid that encodes it and the pharmaceutical carrier(s) or excipient(s). In some embodiments, compositions are prepared by uniformly and intimately bringing into association the active ingredient with liquid carriers or finely divided solid carriers or both, and then, if necessary, shaping the product.

In some embodiments, the administration step comprises intravenous injection, intraperitoneal injection, subcutaneous injection, transdermal injection, or intramuscular injection.

In some embodiments, a composition according to the present disclosure are delivered to a subject suffering from or susceptible to a disease associated with anti-neutrophil antibodies as described herein.

In some embodiments, the subject has or is diagnosed as having ANCA vasculitis. In some embodiments, the individual is a human.

In some embodiments, a subject has or is diagnosed as having anti-PR3 autoantibodies and/or anti-MPO autoantibodies. In some embodiments, a subject having anti-PR3 autoantibodies is treated with a glycoengineered polypeptide or a nucleic acid that encodes it that specifically binds anti-PR3 autoantibodies. In some embodiments, a subject having anti-MPO autoantibodies is treated with a glycoengineered polypeptide or a nucleic acid that encodes it that specifically binds anti-MPO autoantibodies.

In some embodiments, administration of a composition according to the present disclosure alleviates one or more symptoms of ANCA-vasculitis.

In some embodiments, administration of a glycoengineered polypeptide that binds to an anti-PR3 autoantibody treats and/or prevents Granulomatosis with Polyangiitis (GPA)/cytoplasmic ANCA.

In some embodiments, administration of a glycoengineered polypeptide that binds to an anti-MPO autoantibody treats and/or prevents Microscopic Polyangiitis (MPA)/perinuclear ANCA.

### Dosing Regimens

In some embodiments, a method comprises administering a composition once. In some embodiments, a method comprises administering a composition repeatedly (e.g., two or more times over a period of time).

In some embodiments, administration of a composition is continued to maintain remission (e.g., keep anti-neutrophil autoantibodies low and/or undetectable) and/or avoid relapse.

Amounts of glycoengineered polypeptide administered in a single dose may depend on the nature and/or severity of the condition being treated and/or on the nature of prior treatments that the patient has undergone. In some embodiments, the attending physician decides the amount of glycoengineered polypeptide with which to treat each individual patient. In some embodiments, the attending physician initially administers low doses of glycoengineered polypeptides of the present invention and observe the patient's response. In some embodiments, larger doses are administered until an optimal therapeutic effect is obtained for the patient, after which dosage is not increased further.

In some embodiments, a glycoengineered polypeptide according to the present disclosure is delivered in an amount effective to reduce levels of anti-neutrophil autoantibody or a fragment or a complex thereof.

In some embodiments, a glycoengineered polypeptide according to the present disclosure is delivered in an amount effective to reduce levels of anti-PR3 autoantibody or a fragment or a complex thereof.

In some embodiments, a glycoengineered polypeptide according to the present disclosure is delivered in an amount effective to reduce levels of anti-MPO autoantibody or a fragment or a complex thereof.

### Combination Therapies

According to the present disclosure, glycoengineered polypeptides may be administered in combination with one or more therapies. In some embodiments, glycoengineered polypeptides may be administered in combination with one or more pharmaceutical agents. For example, a glycoengineered polypeptide may be administered in combination with one or more therapeutic agents for anti-neutrophil cytoplasmic antibody associated diseases (such as agents that ameliorate symptoms of anti-neutrophil cytoplasmic antibody associated diseases), and/or in combination with one or more other pharmaceutical agents. In some embodiments, glycol-engineered polypeptides may be administered in combination with one or more therapies and/or agents that are prescribed by a clinician for treatment of anti-neutrophil cytoplasmic antibody associated diseases.

In some embodiments, a pharmaceutical composition according to the present disclosure is administered in combination with one or more additional therapies. In some embodiments, a pharmaceutical composition according to the present disclosure is administered in combination with an anti-CD20 antibody, C5a receptor antagonist, an anti-CTLA4-antibody, an anti-CD52 antibody, a BAFF antagonist, glucocorticoids, or combinations thereof. Exemplary therapies that can be used to treat ANCA vasculitis are disclosed in Hillhorst J. et al., (2015) J Am Soc Nephrol 26:2314-2327, the entire contents of which are hereby incorporated by reference. In some embodiments, a pharmaceutical composition according to the present disclosure is administered in combination with one or more therapies and/or agents that are prescribed by a clinician for treatment of anti-neutrophil cytoplasmic antibody associated diseases.

In some embodiments, a pharmaceutical composition according to the present disclosure is administered in combination with an anti-CD20 antibody. In some embodiments, an anti-CD20 antibody is Rituximab.

In some embodiments, a pharmaceutical composition according to the present disclosure is administered in combination with a C5a receptor antagonist. In some embodiments, a C5a receptor antagonist is CCX168.

In some embodiments, a pharmaceutical composition according to the present disclosure is administered in combination with an anti-CTLA4-antibody.

In some embodiments, a pharmaceutical composition according to the present disclosure is administered in combination with an anti-CD52 antibody.

In some embodiments, a pharmaceutical composition according to the present disclosure is administered in combination with a BAFF antagonist.

In some embodiments, a pharmaceutical composition according to the present disclosure is administered in combination with glucocorticoids.

### Characterization of Glycoengineered Polypeptides

The present disclosure provides, among other things, glycoengineered polypeptides that specifically bind to anti-neutrophil autoantibodies (e.g., anti-PR3 autoantibodies and/or anti-MPO autoantibodies) and thereby causing degradation of anti-neutrophil autoantibodies. In some embodiments, anti-neutrophil autoantibodies degradation comprises internalized into a cell for degradation (e.g., by transporting the anti-neutrophil autoantibody to a lysosome). In some embodiments, glycoengineered polypeptides specifically binding anti-neutrophil autoantibodies according to the present disclosure are characterized in that they inhibit the biological function of anti-neutrophil autoantibodies (e.g., binding to neutrophil antigens) including their ability to activate neutrophils.

In some embodiments, a glycoengineered polypeptide according to the present disclosure is used to degrade and/or reduce the levels of anti-neutrophil autoantibodies. In some embodiments, a glycoengineered polypeptide is used to lower anti-neutrophil autoantibody levels, such as lowering elevated plasma anti-neutrophil autoantibody levels in a subject with a disease associated with anti-neutrophil autoantibodies as described herein.

In some embodiments, the present disclosure provides glycoengineered polypeptides characterized in that when administered to a cell, tissue, or subject, the glycoengineered polypeptide which is bound to a target via the first moiety and to an endocytic receptor via a second moiety results in degradation of the target.

In some embodiments, degradation comprises internalization into a cell. In some embodiments, degradation comprises lysosomal degradation. In some embodiments, degradation occurs in a liver cell.

In some embodiments, the present disclosure provides glycoengineered polypeptides characterized in that when administered to a cell, tissue, or subject, the glycoengineered polypeptide which is bound to a target via the first moiety prevents binding of the target to an immune cell. In some embodiments, the immune cell is a neutrophil. In some embodiments, administration of a glycoengineered polypeptide described herein prevents neutrophil activation.

In some embodiments, a target is an anti-PR3 autoantibody or a fragment thereof, and/or an anti-MPO autoantibody or a fragment thereof

In some embodiments, a target comprises an anti-MPO autoantibody or a fragment thereof.

In some embodiments, a target comprises an anti-PR3 autoantibody or a fragment thereof and an anti-MPO autoantibody or a fragment thereof.

In some embodiments, glycoengineered polypeptides disclosed herein are characterized in that when administered to a cell, tissue or subject, glycoengineered polypeptides or compositions comprising the same reduce the level and/or activity of an autoantibody or a fragment thereof (e.g., an anti-MPO autoantibody or a fragment thereof, and/or an anti-PR3 autoantibody or a fragment thereof). In some embodiments, the reduction is as compared to a comparable cell, tissue or subject administered a different therapeutic agent or not administered any therapeutic agent. In some embodiments, the reduction is as compared to the same cell, tissue or subject prior to administration of a glycoengineered polypeptides or compositions comprising the same.

In some embodiments, a level and/or activity of an autoantibody or a fragment thereof is reduced by at least 5%, at least 10%, at least 20%, at least 30%, at least 40%, at least 50%, at least 60%, at least 70%, at least 80%, at least 90%.

In some embodiments, a level and/or activity of an autoantibody or a fragment thereof is reduced by about 5%, about 10%, about 20%, about 30%, about 40%, about 50%, about 60%, about 70%, about 80%, about 90%,

### Method of Making Glycoengineered Polypeptides

The present disclosure, among other things, provides methods of making a glycoengineered polypeptide comprising a first moiety comprising one or more peptides that specifically binds to a target (e.g., an anti-neutrophil autoantibody or a fragment or a complex thereof) and a second moiety comprising one or more glycans conjugated to the first moiety.

A glycoengineered polypeptide disclosed herein can be made using methods disclosed in U.S. Provisional Patent Application No. 63/410,955 filed on Sep. 28, 2022, U.S. Provisional Patent Application No. 63/410,936, filed on Sep. 28, 2022, and International Patent Application PCT/EP2023/076767 filed on Sep. 27, 2023, the entire contents of each of which are hereby incorporated by reference.

For example, in U.S. 63/410,955, Section 5.3 discloses Leishmania host cells; Section 5.4 discloses exemplary methods of genetically engineering a Leishmania cell for expressing glycoengineered polypeptides, Section 5.5 discloses exemplary methods of culturing Leishmania host cells; and Section 5.6 discloses exemplary uses of Leishmania host cells as an expression system.

As another example, in International Patent Application PCT/EP2023/076767, Section 7.1 discloses Leishmania host cells including modifications that can be made to a Leishmania host cell for producing glycoengineered polypeptides, Section 7.2 disclose methods of genetically engineering Leishmania host cells for producing glycoengineered polypeptides, and Section 7.3 discloses methods of culturing Leishmania host cells. An exemplary method of making glycoengineered polypeptides using Leishmania host cells is provided in Example 1 herein.

In particular, as disclosed in PCT/EP2023/076767, exemplary Leishmania strains that can be used to make glycoengineered polypeptides disclosed herein include: StCGP3558, StCGP4564, StCGP5359, or StCGP5942.

As would be understood by persons with ordinary skill in the art, such methods and host cells can also be used for making glycoengineered polypeptides disclosed herein.

In some embodiments, the one or more glycans of the second moiety are conjugated to the first moiety at one or more glycosylation sites with in vivo glycosylation, e.g., in a cell. In some embodiments, a cell is a Leishmania host cell. In some embodiments, a cell is a glycoengineered yeast host cell, e.g., glycoengineered Pichia pastoris host cell.

In some embodiments, the one or more glycans of the second moiety are conjugated to the first moiety at one or more glycosylation sites with chemical conjugation, e.g., using Click chemistry.

Also disclosed herein are methods for making a glycoengineered polypeptide. In one embodiment, provided herein is a method of producing a glycoengineered polypeptide in vivo, using a Leishmania host cell described herein. In some embodiments, provided herein is a method for producing a glycoengineered polypeptide, said method comprising (i) culturing a Leishmania host cell under conditions suitable for polypeptide production and (ii) isolating said glycoengineered polypeptide. In a specific embodiment, the Leishmania host cell comprises: (a) a recombinant nucleic acid encoding a glycoengineered polypeptide; and (b) a recombinant nucleic acid encoding one or more recombinant N-acetylgalactosamine (GalNAc) transferases. In certain embodiments, the Leishmania host cell is capable of producing glycoengineered polypeptide comprising a biantennary, GalNAc-terminated N-glycan. In particular, the Leishmania host cells provided herein is capable of producing glycoengineered polypeptide comprising an N-glycan of the following structure:

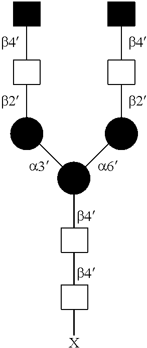

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

In certain embodiments, the glycoengineered polypeptide produced by the Leishmania host cell is a therapeutic polypeptide, i.e., a polypeptide used in the treatment of a disease or disorder. For example, the glycoengineered polypeptide produced by the Leishmania host cell can be peptide or an antibody.

### Leishmania Host Cells

Provided herein are Leishmania host cells for the production of glycoengineered polypeptides disclosed herein, or a population of glycoengineered polypeptides, wherein the Leishmania host cells comprise: (a) a recombinant nucleic acid encoding a glycoengineered polypeptide disclosed herein; and (b) a recombinant nucleic acid encoding one or more recombinant N-acetylgalactosamine (GalNAc) transferases. In certain embodiments, the Leishmania host cells provided herein are capable of producing glycoengineered polypeptides comprising a biantennary, GalNAc-terminated N-glycan. In particular, the Leishmania host cells provided herein are capable of producing glycoengineered polypeptides comprising an N-glycan of the following structure:

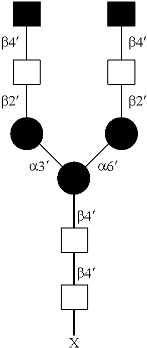

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

In certain embodiments, the Leishmania host cells provided herein comprise a recombinant nucleic acid encoding one or more recombinant N-acetylgalactosamine (GalNAc) transferases disclosed herein. In certain embodiments, the Leishmania host cells provided herein comprise a recombinant nucleic acid encoding one or more additional recombinant glycosyltransferases disclosed herein. In certain embodiments, one or more endogenous enzymes disclosed herein from the glycan biosynthesis pathway of the the Leishmania host cells provided herein have been deleted, mutated and/or functionally inactivated. In certain embodiments, the Leishmania host cells provided herein further comprise a recombinant nucleic acid encoding heterologous UDP-GalNAc biosynthetic pathway proteins capable of generating UDP-GalNAc. In certain embodiments, the Leishmania host cells provided herein comprise a recombinant nucleic acid encoding a heterologous UDP-GalNAc transporter protein capable of transporting UDP-GalNAc to the secretory pathway.

In certain embodiments, the Leishmania host cells provided herein have been genetically engineered such that the formation of an O-linked GlcNAc on a polypeptide produced in the Leishmania host cell is reduced or eliminated. Leishmania host cells that have been genetically engineered to reduce or eliminate the formation of an O-linked GlcNAc are described, for example, in WO 2021/140143, which is incorporated herein by reference in its entirety.x

In certain embodiments, the Leishmania host cells provided herein below are genetically engineered using the methods described herein. In certain embodiments, the Leishmania host cells provided herein below are cultured according to the methods described herein.

Other suitable host cells comprise liver cells, myeloid cells, immune cells, endothelial cells, parenchymal cells or epithelial cells. In some embodiments, the immune cell is a dendritic cell, a macrophage, a monocyte, a microglia cell, a granulocyte or a B lymphocyte.

### Methods of Culturing Leishmania Host Cells

Provided herein are methods for culturing Leishmania host cells. In one embodiment, the Leishmania host cells are cultured using any of the standard culturing techniques known in the art. For example, cells are routinely grown in rich media like Brain Heart Infusion, Trypticase Soy Broth or Yeast Extract, all containing 5 μg/ml Hemin. Additionally, incubation is done at 26° C. in the dark as static or shaking cultures for 2-3 days. In some embodiments, cultures of recombinant cell lines contain the appropriate selective agents. Non-limiting exemplary selective agents are provided in Table 2.

TABLE 1
Selective agents used during transfection (50% concentration for
preselection and 100% concentration for main selection) and standard
culturing of L. tarentolae. Double amounts of the selective agents
could be used if higher selection pressure was intended.

| | | Concentration | | | |
|---|---|---|---|---|---|
| | Resistance | (100%) | | Concentration | |
| Selective | conferring | main selection/ | | (50%) | |
| agent | gene | standard culturing | | preselection | |
| Nourseothricin | sat | 50 | μg/ml | 25 | μg/ml |
| Geneticin | neo | 50 | μg/ml | 25 | μg/ml |
| Paromomycin | neo | 300 | μg/ml | 150 | μg/ml |
| Zeocin | ble | 150 | μg/ml | 75 | μg/ml |
| Hygromycin | hyg | 50 | μg/ml | 25 | μg/ml |
| Blasticidin | bsd | 5 | μg/ml | 2.5 | μg/ml |
| Puromycin | pac | 5 | μg/ml | 2.5 | μg/ml |

In certain embodiments, the Leishmania host cells are cultured in a growth medium comprising GalNAc. In certain embodiments, the growth medium comprises at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, or at least 20 mM GalNAc. In certain embodiments, the growth medium comprises about 1 mM to about 5 mM, about 5 mM to about 10 mM, about 10 mM to about 15 mM, or about 15 mM to about 20 mM GalNAc. In certain embodiments, the growth medium comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM GalNAc. In certain embodiments, the growth medium comprises about about 10 mM GalNAc.

In certain embodiments, the Leishmania host cells are cultured in a growth medium comprising GlcNAc. In certain embodiments, the growth medium comprises at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, or at least 20 mM GlcNAc. In certain embodiments, the growth medium comprises about 1 mM to about 5 mM, about 5 mM to about 10 mM, about 10 mM to about 15 mM, or about 15 mM to about 20 mM GlcNAc. In certain embodiments, the growth medium comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM GlcNAc.

In certain embodiments, a Leishmania host cell may be used as an expression system for making a glycoengineered polypeptide disclosed herein or a population of glycoengineered polypeptides. In certain embodiments, the glycoengineered polypeptide degrader may be a heterologous, non-Leishmania protein, such as a therapeutic protein (e.g., an antibody). Other methods of producing Leishmania host cells for use as expression systems are known and may also be used, for example, see WO 2019/002512, WO 2021/140144 and WO 2021/140143, each of which are incorporated herein by reference in their entirety. Use of Leishmania host cells to make monoclonal antibodies are also known. Exemplary methods are described in WO 2022/053673, which is incorporated herein by reference in its entirety.

The compositions comprising the Leishmania host cells can comprise additional components suitable for maintenance and survival of the Leishmania host cells, and can additionally comprise additional components required or beneficial to the production of glycoengineered bifunctional degraders by the Leishmania host cells, e.g., inducers for inducible promoters, such as arabinose, IPTG.

### Yeast or Filamentous Fungal Host Cells

Provided herein are yeast or filamentous fungal host cells for the production of glycoengineered polypeptides disclosed herein, or a population of glycoengineered polypeptides. In some embodiments, a yeast or filamentous fungal host cell is a K. lactis host cells. In some embodiments, a yeast or filamentous fungal host cell is a Pichia pastoris host cell. In some embodiments, a yeast or filamentous fungal host cell is a Pichia methanolica host cell. In some embodiments, a yeast or filamentous fungal host cell is a Hansenula host cell.

Exemplary yeast or filamentous fungal host cells that can be used to produce glycoengineered polypeptides disclosed herein are disclosed in U.S. Pat. No. 8,206,949, the entire contents of which are hereby incorporated by reference.

Exemplary yeast or filamentous fungal host cells that can be used to produce glycoengineered polypeptides disclosed herein are disclosed in U.S. Pat. No. 7,981,660, the entire contents of which are hereby incorporated by reference.

Exemplary yeast or filamentous fungal host cells that can be used to produce glycoengineered polypeptides disclosed herein are disclosed in U.S. Pat. No. 8,883,483, the entire contents of which are hereby incorporated by reference.

In some embodiments, a yeast or filamentous fungal host cell has been genetically modified to produce glycoproteins with a predominant N-glycan glycoform.

In certain embodiments, the yeast or filamentous fungal host cells provided herein are capable of producing glycoengineered polypeptides comprising a biantennary, GalNAc-terminated N-glycan. In particular, the yeast or filamentous fungal host cells provided herein are capable of producing glycoengineered polypeptides comprising an N-glycan of the following structure:

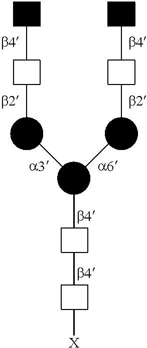

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

### EXAMPLES

### Example 1: Construction of a Glycoengineered Polypeptide of PR3

This example describes the construction and properties of an exemplary glycoengineered polypeptide of PR3 for use in binding to anti-PR3 autoantibodies.

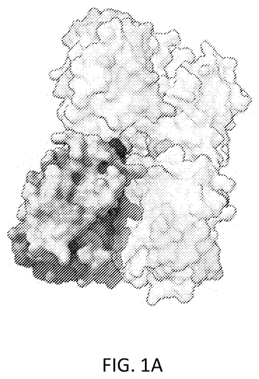

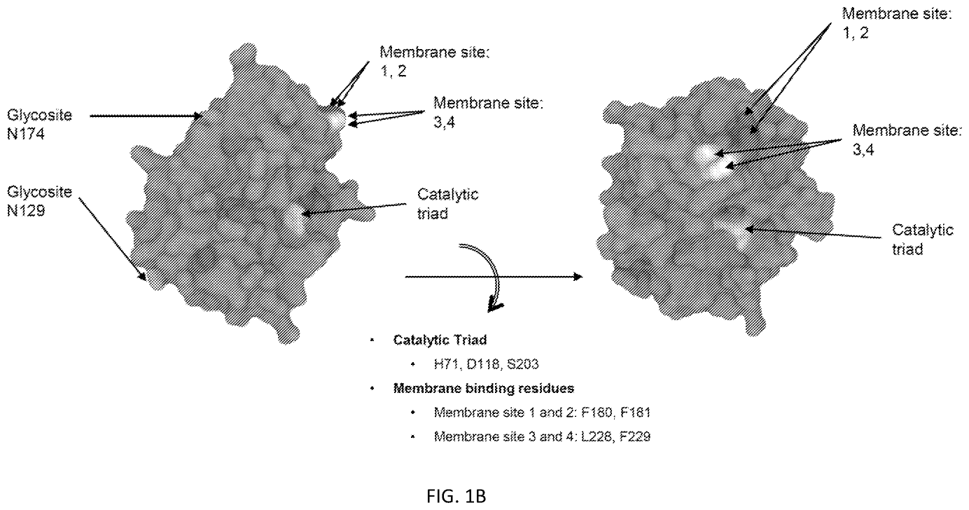

The serine protease enzyme PR3 (FIG. 1A) is one antigen of a subset of anti-neutrophil cytoplasmic (ANCA)-associated Vasculitis, granulomatosis with polyangiitis (Wegener's granulomatosis). PR3 is expressed by neutrophil granulocytes, usually stored in cytoplasm, and externalized during neutrophil apoptosis. The function of PR3 is unknown, but it is implicated in proteolytic generation of antimicrobial peptides during bacterial infections. PR3 can interact with the membrane through the hydrophobic and charged clusters, and these interactions are understood to be important for its proinflammatory functions.

Methods: The construction of the glycoengineered polypeptide of PR3 comprised four major steps. First, due to its protease function, the glycoengineered PR3 was catalytically inactivated. The putative catalytic triad of PR3 is H71, D118, S203 (FIG. 1B; numbering based on Uniprot sequence P24158), and the catalytic inactivation was performed by mutating the S203 reside to Alanine. Previous work has observed that S203A catalytic site inactivation does not impact ANCA binding (Pang, Y.-P. et al. Remote Activation of a Latent Epitope in an Autoantigen Decoded With Simulated B-Factors. Front Immunol 10, 2467 (2019)). Second, a minimum truncation of the native signal peptide was incorporated via the deletion of the N-terminal propeptide of two amino acids (AE). Epitopes of PR3 ANCA are heterogeneous and distributed to multiple sites of the PR3 protein, and therefore the truncated native signal peptide was used at the N-terminus of the glycoengineered PR3. Third, the glycoengineered polypeptide was modified by adding a peptide sequence containing an N-glycosylation consensus site (‘glycotag’) and a C-terminal His tag (FLGT13) for purification. Fourth, due to hydrophobic areas which could potentially result in membrane association, additional sites F180, F181, L228, and F229 were mutated to A, to solubly express recombinant PR3. These construction steps resulted in the PR3 glycoengineered polypeptide protein shown in FIG. 1B (provided herein as SEQ ID NO: 41) having three N-glycosylation sites (two native sites N129 and N174 and a third engineered site within a C-terminal glycotag sequence).

The corresponding nucleotide sequence was transfected as an expression cassette into a glycoengineered Leishmania tarentolae host cell line, as disclosed herein. The resulting cell line stCGP05880 (based on parental strain StCGP4564 disclosed herein) was grown in a bioreactor for PR3 expression and secretion. PR3 glycoengineered polypeptide containing A2GalNAc2 glycans was then purified from the cell supernatant via an IMAC purification and analyzed. Size exclusion chromatography (SEC) was used to measure aggregation and degradation of the protein.

Results: The glycoengineering process resulted in the PR3 glycoengineered polypeptide, having 81.5% A2GalNAc2 (FIG. 1B and Table 2). The purification analysis further demonstrated 80-90% of N-glycosylation sites were fully occupied.

TABLE 2

| | N-glycan | Aggregation/ | Endotoxin | N-glycosylation |
|---|---|---|---|---|
| Protein | (Total) | Degradation (SEC) | (LAL) | site occupancy |
| PR3 | 81.5% | 35.5% monomer | 1.66 EU/mg | 80-90% fully |
| glycoengineered | A2GalNAc2 | 46.9% degradation | | occupied; |
| polypeptide | | 17.7% aggregation | | 10-20% 1 site |
| | | | | unoccupied; <LOD |
| | | | | 2 sites unoccupied |

LOD: limit of detection;
EU: endotoxin units

### Example 2: Glycoengineered PR3 Polypeptides Bind to Anti-PR3 Antibody and Human ASGPR1

This example demonstrates binding of an exemplary glycoengineered polypeptide comprising a PR3 polypeptide to an anti-PR3 antibody and to human ASGPR1.

Methods: Surface plasmon resonance (SPR) was used to measure binding kinetics (i.e., ka, kd, KD, equilibrium) of the PR3 glycoengineered polypeptide analyte to a recombinant CLB12.8 ligand (anti-PR3 antibody) or a biotinylated recombinant human ASGPR1 ligand (Acro Biosystems). Data was collected with a Biocore 8K. Either recombinant CLB12.8 or ASGPR1 was immobilized to the surface of a sensor chip and sequential injections of increasing concentrations of the PR3 glycoengineered polypeptide were performed in a single cycle kinetics experiment. ASGPR1 assays included 2 mM CaCl2 in the running buffer. Data were fit with a 1:1 binding model for CLB12.8 and with a heterogenous binding model for ASGPR1 using the fitting software provided by Cytiva.

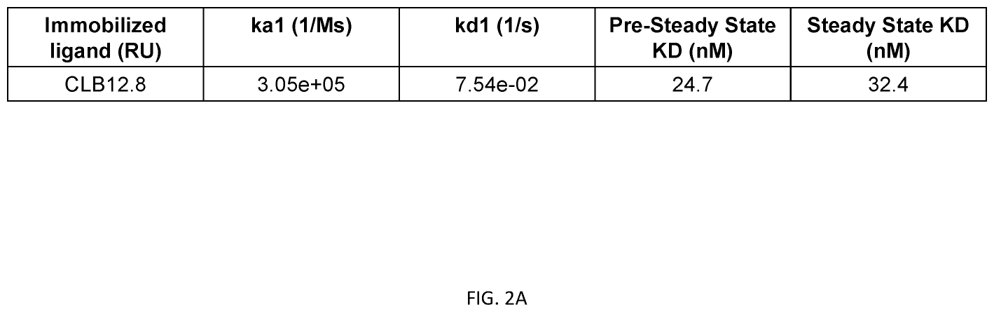

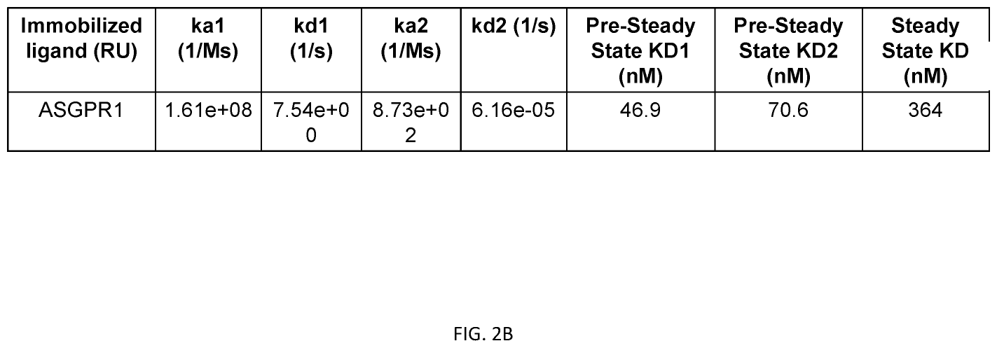

Results: SPR was used to measure the affinity of the PR3 glycoengineered polypeptide to ASGPR1 and CLB12.8 using a single cycle kinetics approach. The PR3 glycoengineered polypeptide displayed an on-rate association with CLB12.8 (ka1) of 3.05×10⁵ 1/Ms, an off-rate, disassociation with CLB12.8 (kd1) of 7.54×10⁻² 1/s, a pre-steady state KD of 24.7 nM, and a steady state KD of 32.4 nM (FIG. 2A). The PR3 glycoengineered polypeptide displayed an on-rate association with ASGPR1 (ka1) of 1.61×10⁸ 1/Ms, an off-rate, disassociation with ASGPR1 (kd1) of 7.54 1/s, a pre-steady state KD1 of 46.9 nM, a pre-steady state KD2 of 70.6 nM, and a steady state KD of 364 nM (FIG. 2B).

This data shows that a PR3 glycoengineered polypeptide binds to a target anti-PR3 antibody and to human ASGPR1. Accordingly, this data supports the development of a glycoengineered polypeptide comprising a PR3 polypeptide and a glycan moiety for binding to anti-PR3 autoantibodies and to an endocytic receptor, e.g., ASGPR1, for targeting anti-PR3 autoantibodies for degradation.

### Example 3: Glycoengineered PR3 Polypeptides Bind to and Block ANCA Autoantibodies

This example demonstrates that an exemplary glycoengineered polypeptide comprising a PR3 polypeptides blocks autoantibodies in PR3+ANCA patient serum.

Methods: Serum from PR3+ANCA patients was acquired and diluted 1:100. Diluted serum was left untreated or incubated with varying concentrations of the PR3 glycoengineered polypeptide or human neutrophil-derived PR3 (HN PR3) for 30 minutes at room temperature. The PR3 glycoengineered polypeptide or HN PR3 concentrations ranged from 0 to 100 μg/ml in the first experiment and 0 to 600 μg/ml in the second experiment. The level of anti-PR3 autoantibodies in the samples were quantified using a c-ANCA ELISA kit following manufacturer's instructions (Catalog #30112111, Tecan/IBL). ELISA data were quantitatively evaluated with a standard curve using a 4-parameter function. Results are expressed as a residual binding measure, calculated by taking the percentage of residual autoantibodies left in the treated sample at a given concentration compared to untreated samples.

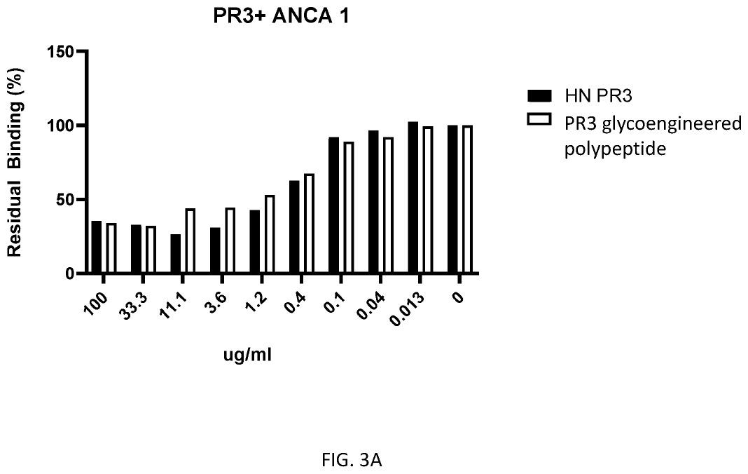

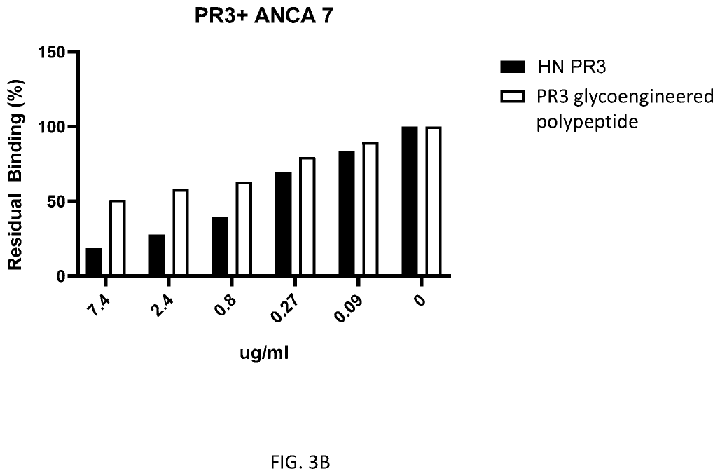

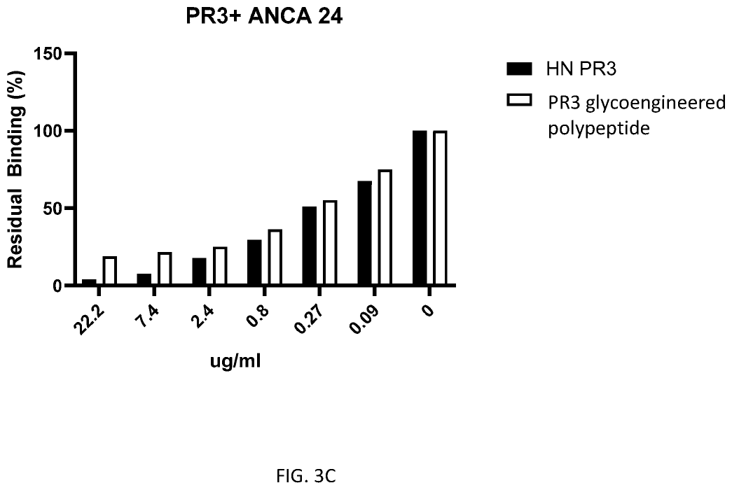

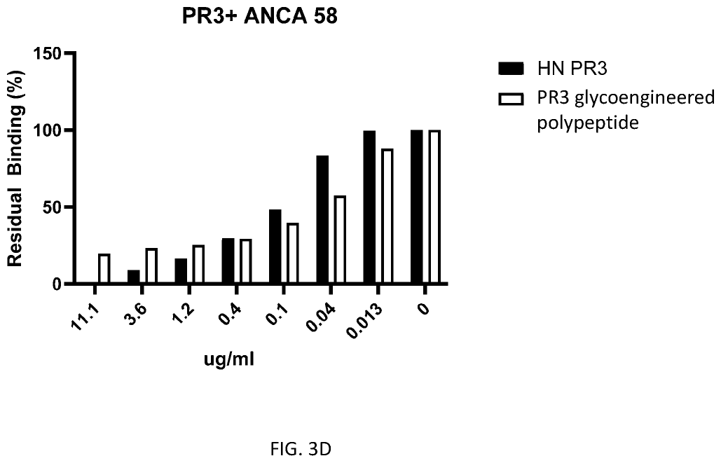

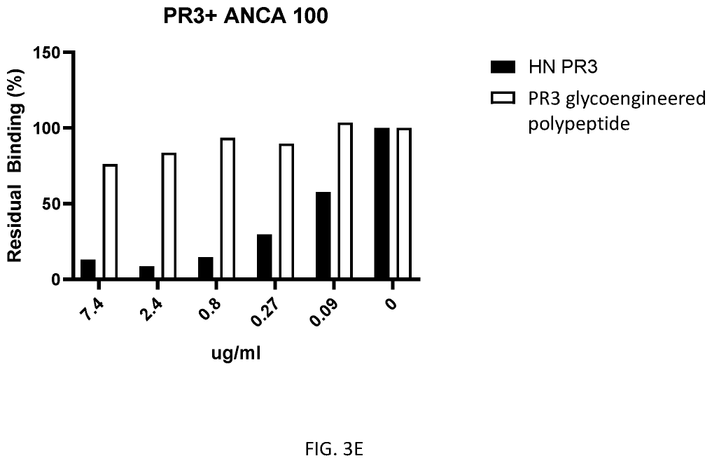

Results: In the first experiment, the PR3 glycoengineered polypeptide reduced the level of anti-PR3 autoantibodies in four out of five PR3+ patient serum samples to approximately 50% or less of the untreated level at one or more concentrations tested (FIGS. 3A-3E; corresponding to patient samples 1, 7, 24, and 58). Furthermore, patient samples 1, 7, 24, and 58 showed comparable residual binding percentage for HN PR3 and the PR3 glycoengineered polypeptide at the majority of concentrations tested (FIGS. 3A-3E; filled versus unfilled bars). In these patient samples, residual binding percentage was dose-dependent, (increasing residual binding with decreasing concentration of the PR3 glycoengineered polypeptide) as was observed with HN PR3. These data indicate that the PR3 glycoengineered polypeptide blocks autoantibodies in PR3+ANCA patient serum.

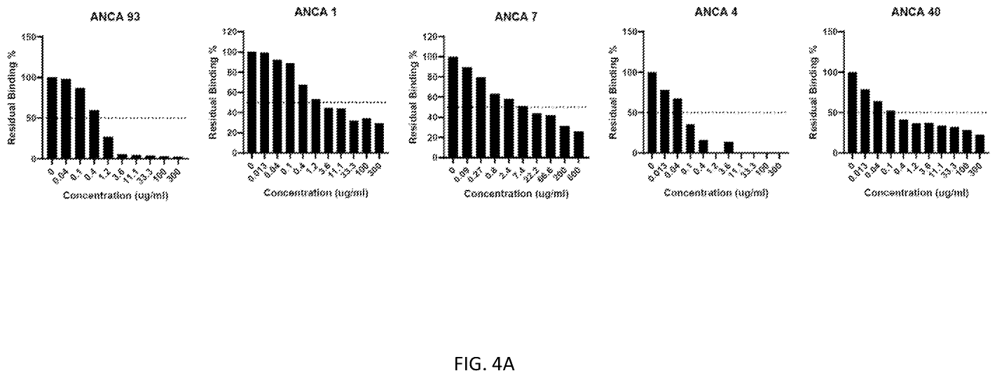

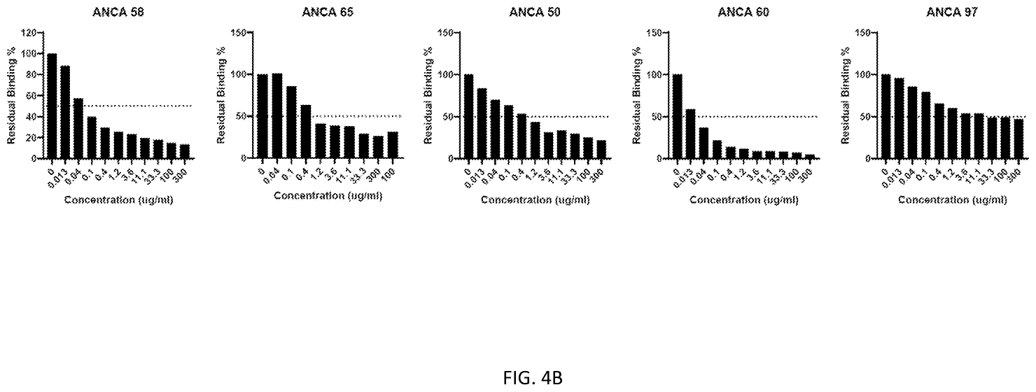

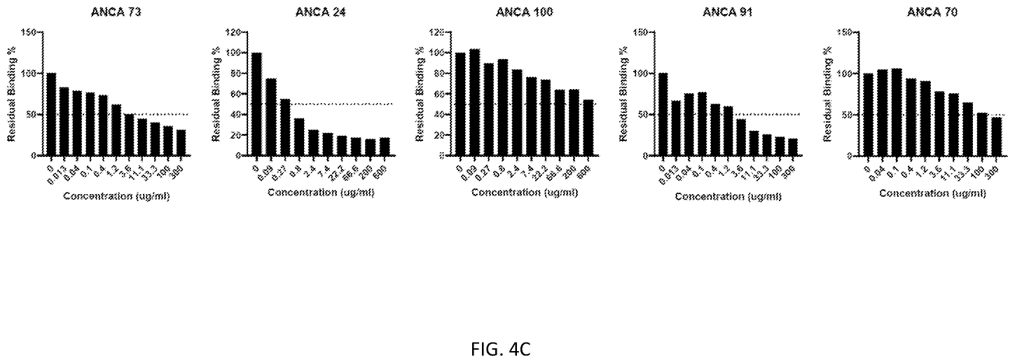

In addition, the PR3 glycoengineered polypeptide reduced the level of anti-PR3 autoantibodies at a high rate in 14 out of 15 PR3+ANCA patient sera. For all ANCA patient samples except the sample from patient 100, a residual binding percentage of 50% or less was achieved at one or more of the concentrations tested (FIG. 4A-C). Residual binding decreased in a concentration-dependent manner in all patient samples. Furthermore, residual binding was not correlated with patient grouping based on initial antibody titer, as reduced residual binding was observed with increasing concentration of the PR3 glycoengineered polypeptide in samples with low autoantibody titer (FIG. 4A), medium autoantibody titer (FIG. 4B) and high autoantibody titer (FIG. 4C). These data are consistent with those of the first experiment and further indicate that the blockade of autoantibodies in ANCA patient samples is independent of the patient serum antibody titer.

This data further supports the development of glycoengineered polypeptides comprising a PR3 polypeptide for use as therapeutic agents in treating and/or preventing ANCA (e.g., in a subject having one or more anti-PR3 autoantibodies). This data also supports the use of glycoengineered polypeptides comprising a PR3 polypeptide for reducing the level of anti-PR3 autoantibodies in a subject.

### Example 4: Depletion of ANCA Autoantibodies with Glycoengineered PR3 Polypeptides

This example demonstrates depletion of anti-PR3 autoantibodies in PR3+ANCA patient serum with an exemplary glycoengineered polypeptide comprising a PR3 polypeptide.

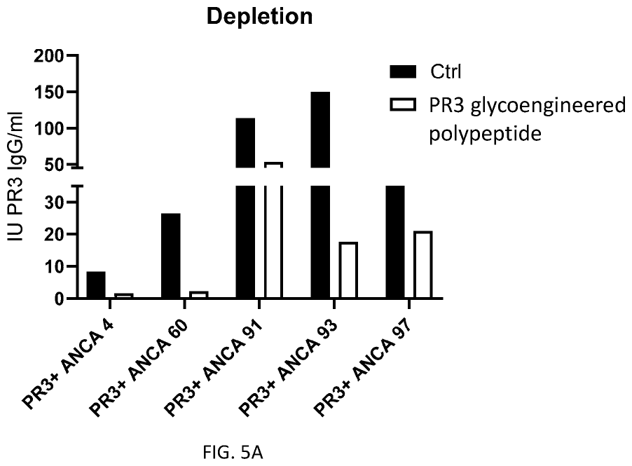

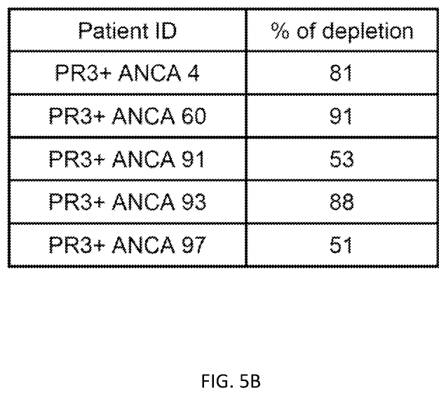

Methods: The PR3 glycoengineered polypeptide or a control protein was coupled to Dynabeads (Catalog #10103D, Invitrogen) following the manufacturer's instructions. Sera was diluted 50 times in 500 ul of Binding/Wash Buffer as per manufacturer protocol, and 1 mg of the PR3 glycoengineered polypeptide or control protein conjugated beads was added to the samples and incubated for 10 minutes at room temperature. Next, tubes were placed on a magnet and supernatant collected (depleted sera). Autoantibody levels were evaluated using c-ANCA ELISA kit following the manufacturer's instructions (Catalog #30112111, Tecan/IBL). ELISA data were then quantitatively evaluated with the standard curve using a 4-parameter function. Results were expressed as international units (IU) of anti-PR3 IgG/ml remaining in the serum (FIG. 5A) or as a percentage of depletion by the PR3 glycoengineered polypeptide (FIG. 5B), calculated as 100−(autoantibodies level in treated sample/autoantibodies level in control sample*100).

Results: Serum from all five patients showed a depletion of anti-PR3 autoantibodies after blocking with the PR3 glycoengineered polypeptide compared to treatment with a control protein (FIG. 5A). For all patients tested, anti-PR3 autoantibodies were depleted by the PR3 glycoengineered polypeptide at a level of 50% or more relative to the control protein (FIG. 5B). This data shows that the PR3 glycoengineered polypeptide but not a control protein depletes anti-PR3 autoantibodies from the serum of PR3+ANCA patients. The percentage of depletion was independent of the starting autoantibodies titers (FIG. 5B,). These data suggest that the PR3 glycoengineered polypeptide can bind to a majority of autoantibodies in ANCA patient serum in solution.

This data supports the development of glycoengineered polypeptides for use as therapeutic agent in treating and/or preventing ANCA (e.g., in a subject having one or more anti-PR3 antibodies). This data further supports the use of glycoengineered polypeptides for reducing the level of anti-PR3 autoantibodies in a subject.

### Example 5: Internalization and Degradation of Glycoengineered PR3 Polypeptides in Complex with Anti-PR3 Antibody in Hepatocytes

This example demonstrates that complexes comprising an exemplary glycoengineered polypeptide and anti-PR3 antibody can be internalized and degraded in hepatocytes.

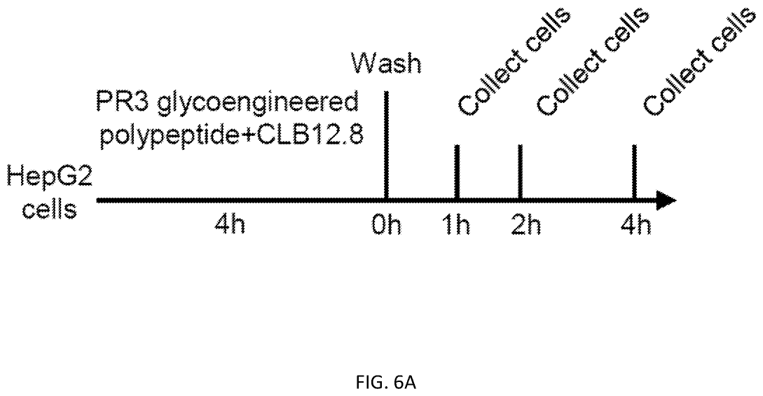

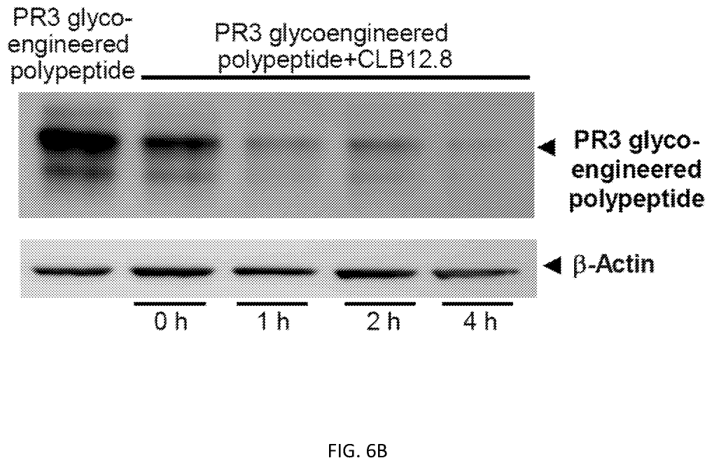

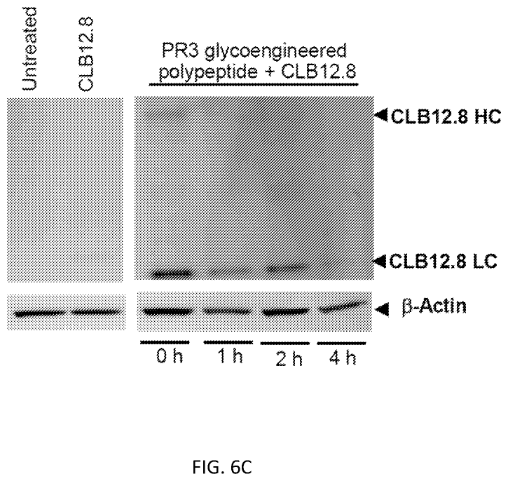

Methods: The experimental set-up is shown in FIG. 6A. The PR3 glycoengineered polypeptide was pre-incubated with an anti-PR3 antibody (clone CLB12.8) and allowed to form complexes. The complexes were added to HepG2 cells for 4 hours to allow internalization. Cells were then washed and collected at 0, 1, 2 and 4 hours post wash. Cell extracts were prepared using RIPA lysis and extraction buffer following the manufacturer instructions (Catalog #89900, ThermoFisherScientific). For internalization analysis, cell extracts were analyzed by immunoblotting using anti-His or anti-mouse IgG antibody. In both analyses, B-actin was used to re-probe the blots as a loading control.

Results: As shown in FIG. 6B, Western blot analysis indicated that the PR3 glycoengineered polypeptide was present in hepatocytes (i.e., internalized) both when added alone and after being allowed to form complexes with the anti-PR3 antibody clone CLB12.8. No change in internalization was observed when the PR3 glycoengineered polypeptide was added in complex to the surrogate autoantibody anti-PR3 clone CLB12.8. After the complexes were washed out from the media, the internalized the PR3 glycoengineered polypeptide was quickly degraded and almost no the PR3 glycoengineered polypeptide was detected in HepG2 cells 4 hours post-wash. (FIG. 6B). These results indicate that the PR3 glycoengineered polypeptide and CLB12.8 complexes were internalized and subsequently degraded in hepatocytes.

Furthermore, the surrogate autoantibody anti-PR3 clone CLB12.8 was only internalized when in complex with the PR3 glycoengineered polypeptide. As shown in FIG. 6C the presence of the anti-PR3 antibody clone CLB12.8 was only detected in blot (i.e., internalized) when CLB12.8 was allowed to form complexes with the PR3 glycoengineered polypeptide (FIG. 6C, compare left panel with right panel). After the complexes were washed out from the media, anti-PR3 clone CLB12.8 was also quickly degraded and almost no remaining antibody was detected 4 hours post wash. These results indicate that the anti-PR3 antibody was internalized only when in complex with the PR3 glycoengineered polypeptide and further show that the internalized complexes were degraded in hepatocytes.

Taken together, the results of this example suggest that the exemplary PR3 glycoengineered polypeptide can promote internalization and degradation of anti-PR3 autoantibodies in hepatocytes. This data further supports the development of glycoengineered polypeptides for use as a therapeutic agent in treating and/or preventing ANCA (e.g., in a subject having one or more anti-PR3 antibodies). This data also supports the development of glycoengineered polypeptides for reducing the level of anti-PR3 autoantibodies in a subject.

### Example 6: Internalization of Glycoengineered PR3 Polypeptides in Complex with Anti-PR3 Antibody is ASGPR-Dependent

This example demonstrates that internalization of an exemplary glycoengineered polypeptide comprising a PR3 polypeptide in complex with an anti-PR3 antibody is dependent on ASGPR.

Methods: The PR3 glycoengineered polypeptide or control material produced in Chinese hamster ovary (CHO) cells was pre-incubated with an anti-PR3 antibody (clone CLB12.8) and allowed to form complexes. The complexes were added to wild-type (WT) HepG2 cells or ASGPR1 knock-out (KO) HepG2 cells for 4 hours to allow internalization. Cells were then washed, and extracts were prepared using RIPA lysis and extraction buffer following the manufacturer instructions (Catalog #89900, ThermoFisherScientific). For internalization analysis, cell extracts were analyzed by immunoblotting using anti-mouse IgG antibody. B-actin was used to re-probe the blots as a loading control.

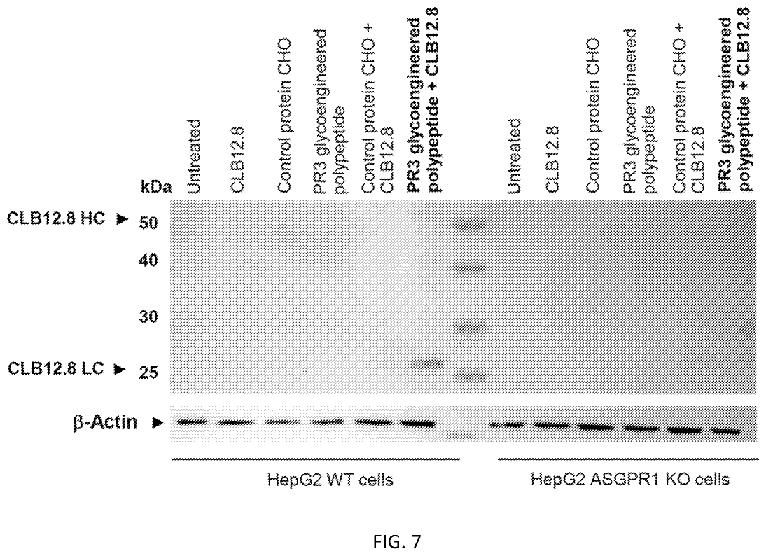

Results: The data shows that only complexes formed with the PR3 glycoengineered polypeptide but not a control protein produced in CHO are internalized in HepG2 cells. Western blot analysis (FIG. 7) detected the presence of CLB12.8 in HepG2 cells only when CLB12.8 was allowed to form complex with the PR3 glycoengineered polypeptide and not when incubated with a control protein produced in CHO cells (FIG. 7, “WT”, left side of blot, sixth column). No internalization was observed in HepG2 cells knock-out for ASGPR, confirming that internalization of the complexes is ASGPR dependent. As shown in FIG. 7, CLB12.8 was not detected when CLB12.8-PR3 glycoengineered polypeptide complexes were added to HepG2 cells where ASGPR was knocked out (FIG. 7, “KO”, right side, last column).

This data indicates that complexes formed with the PR3 glycoengineered polypeptide and an anti-PR3 antibody are preferentially internalized in HepG2 cells compared to complexes of the same antibody and a control protein. Furthermore, this data demonstrates that internalization of the complexes is dependent on ASGPR1.

### Example 7: Construction of a Glycoengineered Polypeptide of MPO

This example describes the construction and properties of an exemplary glycoengineered polypeptide of MPO for use in binding to anti-MPO autoantibodies.

MPO is lysosomal heme enzyme present in the azurophilic granules of human neutrophils and monocytes with microbicidal activity against a wide range of organisms. MPO catalyzes the production of hypohalous acids, primarily hypochlorous acid in physiological conditions, as well as other toxic intermediates that enhance PMN microbicide. MPO undergoes processing and activation resulting in a disulfide-linked homodimer, wherein each monomer consists of a heavy and light chain.

Methods: The construction of the glycoengineered polypeptide of MPO comprised four major steps. First, to maximally cover possible epitopes, the full length MPO apopro variant was used. Second, the putative Cysteine sulfenic acid PTM at C316 (numbering according to Uniprot P05164) was mutated to Alanine. Third, a Leishmania major-derived signal peptide was used at the N-terminus of MPO in place of the native signal peptide, and a His tag was attached to the C-terminus. The native N-glycosylation sites N139, N323, N355, N391, N483, N729 were maintained. Fourth, catalytic inactivation was performed by mutating the H261 proton acceptor active site to Alanine. These construction steps resulted in the MPO glycoengineered polypeptide (SEQ ID NO: 43).

The corresponding nucleotide sequence was transfected as an expression cassette into glycoengineered Leishmania tarentolae host cell line, as disclosed herein. The resulting cell line StCGP 4940 (based on parental strain StCGP4564 disclosed herein) was grown in a bioreactor for MPO expression and secretion. The MPO glycoengineered polypeptide containing A2GalNAc2 glycans was then purified from the cell supernatant via an IMAC purification and analyzed.

Results: The glycoengineering process resulted in the MPO glycoengineered polypeptide with 69.1% A2GalNAc2 (Table 3). The purification analysis further demonstrated more than 90% N-glycosylation site occupancy with no unoccupied sites observed above the limit of detection.

TABLE 3

| | | Aggregation/ | | N-glycosylation |
|---|---|---|---|---|
| Protein | N-glycan | Degradation | Endotoxin | site occupancy |
| MPO | 69.1% | ND | ND | >90% fully occupied |
| glycoengineered | A2GalNAc2 | | | <LOD unoccupied |
| polypeptide | | | | |

ND: not determined;
LOD: limit of detection

### Example 8: Glycoengineered MPO Polypeptides Block ANCA Patient Anti-MPO Autoantibodies

This example demonstrates that an exemplary glycoengineered polypeptide comprising an MPO polypeptide can block autoantibodies in MPO+ANCA patient serum.

Methods: Serum samples from one MPO positive x-ANCA (atypical ANCA) patient were diluted 1:100 and incubated with concentrations of the MPO glycoengineered polypeptide ranging from 0.0001 ug/ml to 10 μg/ml for 30 minutes. Anti-MPO autoantibody levels were measured using an MPO ELISA kit (Catalog #75601, Tecan/IBL) following the manufacturer's instructions. ELISA data were quantitatively evaluated with the standard curve using a 4-parameter function and expressed in international units of anti-MPO IgG/ml.

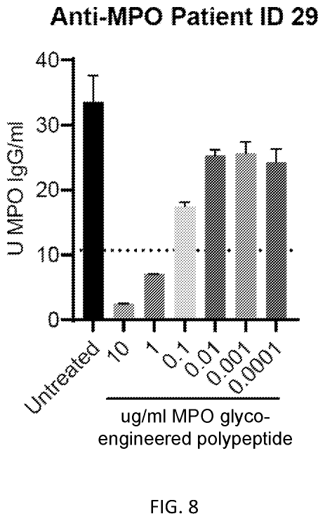

Results: The data shows that the MPO glycoengineered polypeptide blocks the autoantibodies present in MPO+ANCA patient serum. The level of anti-MPO autoantibodies in MPO+ANCA patient serum was reduced compared to untreated samples at all concentrations of the MPO glycoengineered polypeptide tested (FIG. 8). At concentrations of both 1 μg/ml and 10 μg/ml, the MPO glycoengineered polypeptide reduced the level of anti-MPO autoantibodies below the ELISA lower limit of detection (FIG. 8, second and third bars from left).

This data shows that the MPO glycoengineered polypeptide binds and blocks the activity of anti-MPO autoantibodies in MPO+ANCA patient serum.

This data supports the development of glycoengineered polypeptides comprising an MPO polypeptide for use as therapeutic agent in treating and/or preventing ANCA (e.g., in a subject having one or more anti-MPO antibodies). This data further supports the development of glycoengineered polypeptides for reducing the level of anti-MPO autoantibodies in a subject.

### ENUMERATED EMBODIMENTS

Embodiment 1. A glycoengineered polypeptide comprising:
(a) a first moiety comprising one or more peptides that specifically binds to an anti neutrophil autoantibody or a fragment or a complex thereof; and
(b) a second moiety comprising one or more glycans conjugated to the first moiety at one or more glycosylation sites.

Embodiment 2. The glycoengineered polypeptide of embodiment 1, wherein the anti-neutrophil autoantibody is an anti-Proteinase 3 (PR3) autoantibody, or a fragment or a complex thereof.

Embodiment 3. The glycoengineered polypeptide of embodiment 2, wherein the anti-PR3 autoantibody binds to PR3, or a variant or fragment thereof.

Embodiment 4. The glycoengineered polypeptide of embodiment 2 or 3, wherein the anti-PR3 autoantibody binds to PR3 in complex with one or more proteins.

Embodiment 5. The glycoengineered polypeptide of embodiment 4, wherein the one or more proteins in complex with PR3 comprises CD177.

Embodiment 6. The glycoengineered polypeptide of embodiment 1, wherein the anti-neutrophil autoantibody is an anti-Myeloperoxidase (MPO) autoantibody or a fragment or a complex thereof.

Embodiment 7. The glycoengineered polypeptide of embodiment 6, wherein the anti-MPO autoantibody binds to MPO, or a variant or fragment thereof.

Embodiment 8. The glycoengineered polypeptide of embodiment 1, wherein the glycoengineered polypeptide is capable of binding to:
(a) an anti-PR3 autoantibody or a fragment or a complex thereof, and
(b) an anti-MPO autoantibody or a fragment or a complex thereof.

Embodiment 9. The glycoengineered polypeptide of any one of the preceding embodiments, wherein the second moiety specifically binds to one or more endocytic receptors.

Embodiment 10. The glycoengineered polypeptide of embodiment 9, wherein the endocytic receptor is or comprises an endocytic lectin.

Embodiment 11. The glycoengineered polypeptide of embodiment 9 or 10, wherein the endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

Embodiment 12. The glycoengineered polypeptide of embodiment 9 or 10, wherein the endocytic receptor is ASGPR or a fragment or variant thereof, or a complex comprising ASGPR.

Embodiment 13. The glycoengineered polypeptide of any one of the preceding embodiments, wherein the glycan comprises a terminal GlcNac.

Embodiment 14. The glycoengineered polypeptide of any one of embodiments 1-12, wherein the glycan comprises a terminal GalNac.

Embodiment 15. The glycoengineered polypeptide of any one of embodiments 1-12, wherein the glycan comprises a terminal Gal.

Embodiment 16. The glycoengineered polypeptide of any one of the preceding embodiments, wherein the glycan is an N-glycan.

Embodiment 17. The glycoengineered polypeptide of embodiment 16, wherein the N-glycan is linked to the glycoengineered polypeptide at 1, 2, 3, 4 or 5 N-glycosylation sites.

Embodiment 18. The glycoengineered polypeptide of any one of the preceding embodiments, wherein the glycan structure comprises GlcNAc2-Man3-GlcNAc2, GalNAc2-GlcNAc2-Man3-GlcNAc2, Gal2-GlcNAc2-Man3-GlcNAc2, GlcNAc1-Man3-GlcNAc2, Gal2-GlcNAc2-Man3-GlcNAc2, Gal1-GlcNAc2-Man3-GlcNAc2, GalNAc1-GlcNAc2-Man3-GlcNAc2, GlcNAc3-Man3-GlcNAc2, GlcNAc4-Man3-GlcNAc2, Gal3-GlcNAc3-Man3-GlcNAc2, GalNAc3-GlcNAc3-Man3-GlcNAc2, GalNAc4-GlcNAc4-Man3-GlcNAc2, Gal4-GlcNAc4-Man3-GlcNAc2, or Man-6-P—N-glycan.

Embodiment 19. The glycoengineered polypeptide of any one of the preceding embodiments, wherein increasing a number of glycan structures on the glycoengineered polypeptide increases the rate of lysosomal degradation as compared to an otherwise similar glycoengineered polypeptide with fewer glycan structures.

Embodiment 20. The glycoengineered polypeptide of any one of the preceding embodiments, wherein a number of glycan structures comprise 1 or more, 2 or more, 3 or more, 4 or more, 5 or more, 6 or more, 7 or more, 8 or more, 9 or more or 10 or more.

Embodiment 21. The glycoengineered polypeptide of any one of the preceding embodiments, wherein a glycan structure comprises a monoantennary structure, biantennary structure, a triantennary structure, or a tetraantennary structure.

Embodiment 22. The glycoengineered polypeptide of any one of the preceding embodiments, wherein a glycan structure comprises a biantennary structure.

Embodiment 23. The glycoengineered polypeptide of embodiment 22, wherein the glycan structure comprises a biantennary GalNAc.

Embodiment 24. The glycoengineered polypeptide of embodiment 22 or 23, wherein the biantennacy GalNac binds to an asialoglycoprotein receptor (ASGPR) or a fragment or variant thereof, or a complex comprising ASGPR.

Embodiment 25. The glycoengineered polypeptide of any one of embodiments 16-24, wherein the N-glycan has a structure of:

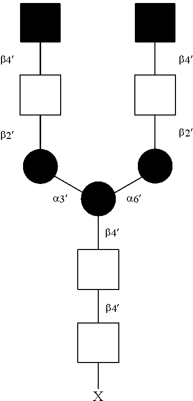

wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.

Embodiment 26. The glycoengineered polypeptide of any one of embodiments 16-25, wherein the N-glycan is conjugated to the glycoengineered polypeptide at at least one, two, three, or four N-glycosylation sites.

Embodiment 27. The glycoengineered polypeptide of any one of embodiments 16-25, wherein the N-glycan is conjugated to the glycoengineered polypeptide at one, two, three, or four N-glycosylation sites.

Embodiment 28. The glycoengineered polypeptide of any one of the preceding embodiments, wherein the N-glycosylation site comprises a consensus sequence of N—X—S/T or N—X—C, wherein X is any amino acid except proline.

Embodiment 29. The glycoengineered polypeptide of any one of the preceding embodiments, wherein the N-glycosylation site is naturally occurring.

Embodiment 30. The glycoengineered polypeptide of any one of embodiments 1-28, wherein the N-glycosylation site is engineered into the amino acid sequence of the first moiety, optionally wherein the engineered N-glycosylation site comprises the sequence of GGGGANSTAPAPAPA (SEQ ID NO: 37).

Embodiment 31. The glycoengineered polypeptide of any one of the preceding embodiments, wherein the endocytic receptor is or comprises ASGPR or a fragment or variant thereof.

Embodiment 32. The glycoengineered polypeptide of embodiment 31, wherein when the endocytic receptor is ASGPR, the glycan structure of the second moiety comprises a terminal GalNac.

Embodiment 33. The glycoengineered polypeptide of any one of the preceding embodiments, wherein the glycoengineered polypeptide comprises a first moiety comprising one or more peptides that specifically binds to an anti-PR3 autoantibody or a fragment thereof.

Embodiment 34. The glycoengineered polypeptide of embodiment 33, wherein the first moiety comprises 1, 2, 3, 4, 5, or more peptides that specifically bind to an anti-PR3 autoantibody.

Embodiment 35. The glycoengineered polypeptide of embodiment 33 or 34, wherein the one or more peptides that specifically bind to an anti-PR3 autoantibody are the same.

Embodiment 36. The glycoengineered polypeptide of embodiment 35, wherein the one or more peptides that specifically bind to an anti-PR3 autoantibody are separated by an intervening sequence.

Embodiment 37. The glycoengineered polypeptide of embodiment 36, wherein the intervening sequence is an IRES, a protease cleavage site, a linker or a spacer or a combination thereof.

Embodiment 38. The glycoengineered polypeptide of embodiment 33 or 34, wherein the one or more polypeptides that specifically bind to an anti-PR3 autoantibody are different.

Embodiment 39. The glycoengineered polypeptide of embodiment 38, wherein the one or more peptides that specifically bind to an anti-PR3 autoantibody are separated by an intervening sequence.

Embodiment 40. The glycoengineered polypeptide of embodiment 39, wherein the intervening sequence is an IRES, a protease cleavage site, a linker or a spacer or a combination thereof.

Embodiment 41. The glycoengineered polypeptide of any one of embodiments 38-40, wherein the different peptides form a spatial epitope.

Embodiment 42. The glycoengineered polypeptide of any one of embodiments 33-41, wherein the one or more peptides that specifically bind to an anti-PR3 autoantibody are each conjugated to a second moiety.

Embodiment 43. The glycoengineered polypeptide of any one of embodiments 33-41, wherein the one or more peptides that specifically bind to an anti-PR3 autoantibody are not each conjugated to the second moiety.

Embodiment 44. The glycoengineered polypeptide of any one of embodiments 33-43, wherein the one or more peptides that specifically bind to an anti-PR3 autoantibody are conjugated to each other.

Embodiment 45. The glycoengineered polypeptide of embodiment 44, wherein the one or more peptides are situated on one polypeptide.

Embodiment 46. The glycoengineered polypeptide of embodiment 44 or 45, wherein the one or more peptides are separated by an intervening amino acid sequence.

Embodiment 47. The glycoengineered polypeptide of embodiment of embodiment 46, wherein the intervening amino acid sequence is an IRES, a protease cleavage site, a linker or a spacer or a combination thereof.

Embodiment 48. The glycoengineered polypeptide of any one of embodiments 33-47, wherein the glycoengineered polypeptide comprises a first moiety comprising one peptide that specifically binds to an anti-PR3 autoantibody or a fragment thereof.

Embodiment 49. The glycoengineered polypeptide of any one of embodiments 33-48, wherein the one or more peptides that specifically bind to an anti-PR3 autoantibody is a soluble polypeptide.

Embodiment 50. The glycoengineered polypeptide of any one of embodiments 33-49, wherein the one or more peptides that specifically bind to an anti-PR3 autoantibody comprises a PR3 protein, or a fragment or a variant thereof.

Embodiment 51. The glycoengineered polypeptide of embodiment 50, wherein the PR3 protein is provided as SEQ ID NO: 1 with or without the signal peptide, SEQ ID NO: 40 with or without the signal peptide, or SEQ ID NO: 45.

Embodiment 52. The glycoengineered polypeptide of embodiment 50 or 51, wherein the one or more polypeptides that specifically bind to an anti-PR3 autoantibody comprises a fragment of a PR3 protein.

Embodiment 53. The glycoengineered polypeptide of embodiment 52, wherein the fragment comprises at least 5% of a full length PR3 protein with or without the signal peptide.

Embodiment 54. The glycoengineered polypeptide of embodiment 52, wherein the fragment comprises no more than 99% of a full length PR3 protein with or without the signal peptide.

Embodiment 55. The glycoengineered polypeptide of any one of embodiments 52-54, wherein the fragment comprises an epitope that is recognized by a PR3 autoantibody.

Embodiment 56. The glycoengineered polypeptide of embodiment 55, wherein the epitope is a linear epitope.

Embodiment 57. The glycoengineered polypeptide of embodiment 55, wherein the epitope is a conformational epitope.

Embodiment 58. The glycoengineered polypeptide of embodiment 57, wherein the conformational epitope comprises one or more peptides that form the epitope spatially.

Embodiment 59. The glycoengineered polypeptide of any one of embodiments 55-58, wherein the epitope comprises SEQ ID NO: 2 or SEQ ID NO: 3, or a portion of SEQ ID NO:2 or SEQ ID NO: 3.

Embodiment 60. The glycoengineered polypeptide of any one of embodiments 55-58, wherein the epitope comprises any one of SEQ ID NOs: 11, 12, 13, 14, 15, 16, 17, 18, 19, 20, 21, 22, 23, or a portion of any of the aforementioned SEQ ID NOS.

Embodiment 61. The glycoengineered polypeptide of any one of embodiments 55-60, wherein the epitope comprises one or more additional amino acid residues on the 5′ and/or 3′ end of the sequence.

Embodiment 62. The glycoengineered polypeptide of any one of embodiments 33-60, wherein the one or more polypeptides that specifically bind to an anti-PR3 autoantibody is a variant of a PR3 protein.

Embodiment 63. The glycoengineered polypeptide of embodiment 62, wherein the variant is an inactive variant as compared to a wild-type PR3 protein.

Embodiment 64. The glycoengineered polypeptide of embodiment 62 or 63, wherein the variant comprises a mutation at the Valine residue at position 119, the Alanine residue at position 135, the Threonine residue at position 136, or a combination thereof.

Embodiment 65. The glycoengineered polypeptide of embodiment 64, wherein the mutation is:
(a) a Valine to Isoleucine mutation;
(b) an Alanine to Threonine mutation; and/or
© is a Serine to Threonine mutation.

Embodiment 66. The glycoengineered polypeptide of any one of embodiments 62-65, wherein the variant comprises a mutation at one or more or all of amino acids 71 (His), 118 (Asp) and 203 (Ser) of SEQ ID NO: 1.

Embodiment 67. The glycoengineered polypeptide of any one of embodiments 62-66, wherein the variant comprises a mutation at one or more or all of amino acids 180 (Phe), 181 (Phe), 228 (Leu), or 229 (Phe) of SEQ ID NO: 1.

Embodiment 68. The glycoengineered polypeptide of any one of embodiments 33-67, wherein the one or more polypeptides that specifically bind to an anti-PR3 autoantibody comprises a contiguous chain of amino acids comprising at least 5% of the amino acid of SEQ ID NO: 1, SEQ ID NO: 40 or SEQ ID NO: 45.

Embodiment 68.1 The glycoengineered polypeptide of any one of the preceding embodiments, wherein the one or more peptides that specifically bind to an anti-PL3 autoantibody comprises a sequence having at least 85% identity to:
(i) SEQ ID NO: 1; or
(ii) SEQ ID NO: 1 without the signal peptide, optionally wherein the sequence further comprises a different signal peptide, e.g., as disclosed herein.

Embodiment 68.2. The glycoengineered polypeptide of any one of embodiments 1-68, wherein the one or more peptides that specifically bind to an anti-PL3 autoantibody comprises a sequence having at least 85% identity to:
(i) SEQ ID NO: 40; or
(ii) SEQ ID NO: 40 without the signal peptide, optionally wherein the sequence further comprises a different signal peptide, e.g., as disclosed herein.

Embodiment 68.3 The glycoengineered polypeptide of any one of embodiments 1-68, wherein the one or more peptides that specifically bind to an anti-PL3 autoantibody comprises a sequence having at least 85% identity to SEQ ID NO: 45, optionally wherein the sequence further comprises a signal peptide, e.g., as disclosed herein.

Embodiment 68.4 The glycoengineered polypeptide of any one of embodiments 1-68, wherein the one or more peptides that specifically bind to an anti-PL3 autoantibody comprises a sequence having at least 85% identity to SEQ ID NO: 41.

Embodiment 69. The glycoengineered polypeptide of any one of embodiments 33-68, wherein the one or more polypeptides that specifically bind to an anti-PR3 autoantibody comprises a full length PR3 protein.

Embodiment 70. The glycoengineered polypeptide of any one of embodiments 33-69, wherein the one or more polypeptides that specifically bind to an anti-PR3 autoantibody comprises an antibody agent.

Embodiment 71. The glycoengineered polypeptide of embodiment 70, wherein the antibody agent comprises an antigen binding fragment.

Embodiment 72. The glycoengineered polypeptide of embodiment 71 wherein the antibody agent comprises a full antibody, a Fab fragment, an scFv, a nanobody, duobody or a single domain antibody (e.g., a VHH).

Embodiment 73. The glycoengineered polypeptide of any one of embodiments 1-32, wherein the glycoengineered polypeptide comprises a first moiety comprising one or more peptides that specifically binds to an anti-MPO autoantibody or a fragment thereof.

Embodiment 74. The glycoengineered polypeptide of embodiment 73, wherein the first moiety comprises 1, 2, 3, 4, 5, or more peptides that specifically bind to an anti-MPO autoantibody.

Embodiment 75. The glycoengineered polypeptide of embodiment 72 or 73, wherein the one or more peptides that specifically bind to an anti-MPO autoantibody are the same.

Embodiment 76. The glycoengineered polypeptide of embodiment 75, wherein the one or more peptides that specifically bind to an anti-MPO autoantibody are separated by an intervening sequence.

Embodiment 77. The glycoengineered polypeptide of embodiment 76, wherein the intervening sequence is an IRES, a protease cleavage site, a linker or a spacer or a combination thereof.

Embodiment 78. The glycoengineered polypeptide of embodiment 72 or 73, wherein the one or more peptides that specifically bind to an anti-MPO autoantibody are different.

Embodiment 79. The glycoengineered polypeptide of embodiment 78, wherein the one or more peptides that specifically bind to an anti-MPO autoantibody are separated by an intervening sequence.

Embodiment 80. The glycoengineered polypeptide of embodiment 79, wherein the intervening sequence is an IRES, a protease cleavage site, a linker or a spacer or a combination thereof.

Embodiment 81. The glycoengineered polypeptide of any one of embodiments 78-80, wherein the different peptides form a spatial epitope.

Embodiment 82. The glycoengineered polypeptide of embodiment 73-81, wherein the one or more peptides that specifically bind to an anti-MPO autoantibody are each conjugated to a second moiety.

Embodiment 83. The glycoengineered polypeptide of embodiment 73-81, wherein the one or more peptides that specifically bind to an anti-MPO autoantibody are not each conjugated to the second moiety.

Embodiment 84. The glycoengineered polypeptide of embodiment 83, wherein the one or more peptides that specifically bind to an anti-MPO autoantibody are conjugated to each other.

Embodiment 85. The glycoengineered polypeptide of embodiment 83 or 84, wherein the one or more peptides are separated by an intervening amino acid sequence.

Embodiment 86. The glycoengineered polypeptide of embodiment of embodiment 85, wherein the intervening amino acid sequence is an IRES, a protease cleavage site, a linker or a spacer or a combination thereof.

Embodiment 87. The glycoengineered polypeptide of any one of embodiments 73-86, wherein the glycoengineered polypeptide comprises a first moiety comprising one peptide that specifically binds to an anti-MPO autoantibody or a fragment thereof.

Embodiment 88. The glycoengineered polypeptide of any one of embodiments 73-87, wherein the one or more peptides that specifically bind to an anti-MPO autoantibody is a soluble polypeptide.

Embodiment 89. The glycoengineered polypeptide of any one of embodiments 73-88, wherein the one or more peptides that specifically bind to an anti-MPO autoantibody comprise a MPO polypeptide, or a fragment or a variant thereof.

Embodiment 90. The glycoengineered polypeptide of embodiment 89, wherein the MPO polypeptide is provided as SEQ ID NO: 4 with or without the signal peptide, SEQ ID NO: 42 with or without the signal peptide, or SEQ ID NO: 46.

Embodiment 91. The glycoengineered polypeptide of embodiment 88 or 89, wherein the one or more peptides that specifically bind to an anti-MPO autoantibody comprise a fragment of an MPO polypeptide.

Embodiment 92. The glycoengineered polypeptide of embodiment 91, wherein the fragment comprises at least 5% of a full length MPO polypeptide with or without the signal peptide.

Embodiment 93. The glycoengineered polypeptide of embodiment 91, wherein the fragment comprises no more than 99% of a full length MPO polypeptide with or without the signal peptide.

Embodiment 94. The glycoengineered polypeptide of any one of embodiments 91-93, wherein the fragment comprises an epitope that is recognized by a MPO autoantibody.

Embodiment 95 . . . . The glycoengineered polypeptide of embodiment 94, wherein the epitope is a linear epitope.

Embodiment 96. The glycoengineered polypeptide of embodiment 94, wherein the epitope is a conformational epitope.

Embodiment 97. The glycoengineered polypeptide of embodiment 95, wherein the conformational epitope comprises one or more peptides that form the epitope spatially.

Embodiment 98. The glycoengineered polypeptide of any one of embodiments 94-97, wherein the epitope comprises SEQ ID NO: 5 or SEQ ID NO: 10, or a portion of SEQ ID NO:5 or SEQ ID NO: 10.

Embodiment 99. The glycoengineered polypeptide of any one of embodiments 94-97, wherein the epitope comprises any one of SEQ ID NOs: 24, 25, 26, 27, 28, 29, 30, 31, 32, 33, 34, or a portion of any of the aforesaid SEQ ID NOs.

Embodiment 100. The glycoengineered polypeptide of any one of embodiments 94-99, wherein the epitope comprises one or more additional amino acid residues on the 5′ and/or 3′ end of the sequence.

Embodiment 101. The glycoengineered polypeptide of any one of embodiments 73-100, wherein the one or more peptides that specifically bind to an anti-MPO autoantibody is a variant of an MPO polypeptide, optionally wherein the variant is an inactive variant as compared to a wild-type MPO protein.

Embodiment 102. The glycoengineered polypeptide of embodiment 101, wherein the variant comprises a mutation at one or more or all of 261 (His), 316 (Cys), 405 (Arg) and 257 (Gln) of SEQ ID NO: 4.

Embodiment 103. The glycoengineered polypeptide of any one of embodiments 73-102, wherein the one or more peptides that specifically bind to an anti-MPO autoantibody comprises a contiguous chain of amino acids comprising at least 5% of the amino acid of SEQ ID NO: 4, SEQ ID NO: 42 or SEQ ID NO: 46.

Embodiment 103.1 The glycoengineered polypeptide of any one of embodiments 73-103, wherein the one or more peptides that specifically bind to an anti-MPO autoantibody comprises a sequence having at least 85% identity to:
(i) SEQ ID NO: 4; or
(ii) SEQ ID NO: 4 without the signal peptide, optionally wherein the sequence further comprises a different signal peptide, e.g., as disclosed herein.

Embodiment 103.2 The glycoengineered polypeptide of any one of embodiments 73-103, wherein the one or more peptides that specifically bind to an anti-MPO autoantibody comprises a sequence having at least 85% identity to:
(i) SEQ ID NO: 42; or
(ii) SEQ ID NO: 42 without the signal peptide, optionally wherein the sequence further comprises a different signal peptide, e.g., as disclosed herein.

Embodiment 103.3. The glycoengineered polypeptide of any one of embodiments 73-103, wherein the one or more peptides that specifically bind to an anti-MPO autoantibody comprises a sequence having at least 85% identity to SEQ ID NO: 46, optionally wherein the sequence further comprises a signal peptide, e.g., as disclosed herein.

Embodiment 103.4. The glycoengineered polypeptide of any one of embodiments 73-103, wherein the one or more peptides that specifically bind to an anti-MPO autoantibody comprises a sequence having at least 85% identity to SEQ ID NO: 43.

Embodiment 104. The glycoengineered polypeptide of any one of embodiments 73-103, wherein the one or more peptides that specifically bind to an anti-MPO autoantibody comprise a full length MPO protein.

Embodiment 10⁵. The glycoengineered polypeptide of any one of embodiments 73-104, wherein the one or more peptides that specifically bind to an anti-MPO autoantibody comprise an antibody agent.

Embodiment 106. The glycoengineered polypeptide of embodiment 10⁵, wherein the antibody agent comprises an antigen binding fragment.

Embodiment 107. The glycoengineered polypeptide of any one of embodiments 10⁵-106, wherein the antibody agent comprises a full antibody, a Fab fragment, an scFv, a nanobody, a duobody, or a single domain antibody (e.g., a VHH).

Embodiment 108. The glycoengineered polypeptide of any one of embodiments 73-107, wherein the one or more peptides of the first moiety binds to the E3 anti-MPO autoantibody.

Embodiment 109. The glycoengineered polypeptide of any one of the preceding embodiments, wherein the first moiety comprises (i) one or more anti-PR3 autoantibody binding polypeptides and (ii) one or more anti-MPO autoantibody binding polypeptides.

Embodiment 110. The glycoengineered polypeptide of embodiment 109, wherein the one or more anti-PR3 autoantibody binding polypeptides and the one or more anti-MPO autoantibody binding polypeptides are situated on the same polypeptide.

Embodiment 111. The glycoengineered polypeptide of 108 or 109, wherein the one or more anti-PR3 autoantibody binding polypeptides and the one or more anti-MPO autoantibody binding polypeptides are situated on different polypeptides.

Embodiment 112. The glycoengineered polypeptide of any one of embodiments 108-111, wherein the one or more anti-PR3 autoantibody binding polypeptides are situated on a first glycoengineered polypeptide and the one or more anti-MPO autoantibody binding polypeptides are situated on a second glycoengineered polypeptide.

Embodiment 113. The glycoengineered polypeptide of any one of embodiments 108-112, wherein each of the anti-PR3 autoantibody binding polypeptides and each of the anti-MPO autoantibody binding polypeptides are conjugated to a second moiety.

Embodiment 114. The glycoengineered polypeptide of any one of the preceding embodiments, wherein the polypeptide comprises one or more additional elements chosen from:
(a) a linker,
(b) a spacer,
(c) a cleavage peptide, e.g., an IRES or a protease cleavage site,
(d) a signal peptide,
(e) a tag, e.g., a cleavable tag,
(f) a half-life extender domain, e.g., an Fc domain or albumin,
(g) any combination of (a)-(f).

Embodiment 115. The glycoengineered polypeptide of any one of the preceding embodiments, wherein the second moiety is conjugated to the first moiety at least one, two, three, or four N-glycosylation sites.

Embodiment 116. The glycoengineered polypeptide of any one of embodiments 1-114, wherein the second moiety is conjugated to the first moiety at one, two, three, or four N-glycosylation sites.

Embodiment 117. The glycoengineered polypeptide of any one of the preceding embodiments, wherein the N-glycosylation site comprises a consensus sequence of N—X—S/T or N—X—C, wherein X is any amino acid except proline.

Embodiment 118. The glycoengineered polypeptide of any one of the preceding embodiments, wherein the second moiety is conjugated to the first moiety in vivo.

Embodiment 119. The glycoengineered polypeptide of embodiment 118, wherein the conjugation occurs in a cell.

Embodiment 120. The glycoengineered polypeptide of embodiment 119, wherein the cell is a Leishmania cell.

Embodiment 121. The glycoengineered polypeptide of any one of embodiments 1-117, wherein the second moiety is conjugated to the first moiety by chemical conjugation.

Embodiment 122. The glycoengineered polypeptide of embodiment 121, wherein chemical conjugation comprises click chemistry.

Embodiment 123. The glycoengineered polypeptide of any one of the preceding embodiments, wherein the first moiety is an antibody and the antibody is a monoclonal or polyclonal antibody.

Embodiment 124. The glycoengineered polypeptide of embodiment 123, wherein the antibody is a recombinant antibody.

Embodiment 125. The glycoengineered polypeptide of embodiment 124, wherein the antibody is humanized, chimeric or fully human.

Embodiment 126. The glycoengineered polypeptide of embodiment 125, wherein the antibody has a glycan to protein ratio of 2 to 1, 4 to 1, 6 to 1, 8 to 1, or 10 to 1.

Embodiment 127. The glycoengineered polypeptide of any one of embodiments 123-126, wherein the antibody is glycosylated at a predetermined and specific residue.

Embodiment 128. The glycoengineered polypeptide of any one of the preceding embodiments, characterized in that when administered to a cell, tissue, or subject, the glycoengineered polypeptide which is bound to a target via the first moiety and to an endocytic receptor via a second moiety results in degradation of the target.

Embodiment 129. The glycoengineered polypeptide of embodiment 128, wherein the target is an anti-PR3 autoantibody or a fragment thereof, and/or an anti-MPO autoantibody or a fragment thereof.

Embodiment 130. The glycoengineered polypeptide of embodiment 129, wherein degradation comprises internalization into a cell.

Embodiment 131. The glycoengineered polypeptide of embodiment 129 or 130, wherein the target and the glycoengineered polypeptide are internalized.

Embodiment 132. The glycoengineered polypeptide of any one of embodiments 128-131, wherein degradation comprises lysosomal degradation.

Embodiment 133. The glycoengineered polypeptide of embodiment 132, wherein degradation occurs in a liver cell.

Embodiment 134. The glycoengineered polypeptide of any one of embodiments of the preceding embodiments, characterized in that when administered to a cell, tissue, or subject the glycoengineered polypeptide which is bound to a target via the first moiety prevents binding of the target to an immune cell.

Embodiment 135. The glycoengineered polypeptide of embodiment 134, wherein prevention of binding of the target to an immune cell prevents activation of the immune cell.

Embodiment 136. The glycoengineered polypeptide of embodiment 135, wherein the immune cell is a neutrophil.

Embodiment 137. A polynucleotide encoding the glycoengineered polypeptide of any one of the preceding embodiments.

Embodiment 138. The polynucleotide of embodiment 137, wherein the polynucleotide comprises a portion of the nucleotide sequence of SEQ ID NO: 7 or a codon-optimized version thereof.

Embodiment 139. The polynucleotide of embodiment 138, wherein the polynucleotide comprises at least 10% of SEQ ID NO: 7 or a codon-optimized version thereof.

Embodiment 140. The polynucleotide of embodiment 137, wherein the polynucleotide comprises a portion of the nucleotide sequence of SEQ ID NO: 9 or a codon-optimized version thereof.

Embodiment 141. The polynucleotide of embodiment 140, wherein the polynucleotide comprises at least 10% of SEQ ID NO: 9, or a codon-optimized version thereof.

Embodiment 142. A composition comprising a glycoengineered polypeptide of any one of the preceding embodiments.

Embodiment 143. The composition of embodiment 142, wherein the composition comprises a glycoengineered polypeptide comprising a first moiety that specifically binds to an anti-PR3 autoantibody or a fragment or a complex thereof.

Embodiment 144. The composition of embodiment 142 wherein the composition comprises a glycoengineered polypeptide comprising a first moiety that specifically binds to an anti-MPO autoantibody or a fragment or a complex thereof.

Embodiment 145. The composition of embodiment 142, wherein the composition comprises:
(i) a first glycoengineered polypeptide comprising a first moiety that specifically binds to an anti-PR3 autoantibody or a fragment or a complex thereof, and
(ii) a second glycoengineered polypeptide comprising a first moiety that specifically binds to an anti-MPO autoantibody or a fragment or a complex thereof.

Embodiment 146. The composition of embodiment 145, wherein the ratio of the first glycoengineered polypeptide to the second glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

Embodiment 147. The composition of embodiment 145, wherein the ratio of the first glycoengineered polypeptide to the second glycoengineered polypeptide is about 1:5 to about 5:1; about 1:2.5 to about 2.5:1.

Embodiment 148. The composition of embodiment 145, wherein the ratio of the first glycoengineered polypeptide to the second glycoengineered polypeptide is about 1:1.5 to about 1.5:1.

Embodiment 149. The composition of any one of embodiments 145-148, wherein the first glycoengineered polypeptide is present at an amount of about 10-90% and the second glycoengineered polypeptide is present at an amount of about 90-10%.

Embodiment 150. A composition comprising a population of glycoengineered polypeptides of any one of embodiments 1-136, wherein the population of glycoengineered polypeptides has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s).

Embodiment 151. The composition of embodiment 150, wherein the homogeneity of the N-glycan profile at one or more of the N-glycosylation sites is determined by N-glycan analysis, glycopeptide analysis or intact protein analysis.

Embodiment 152. The composition of embodiment 151, wherein the N-glycan profile comprises about 30% to 40%, about 40% to about 50%, about 50% to about 60%, about 60% to about 70%, about 70% to about 80%, about 80% to about 90%, or about 90% to about 100% of the N-glycan of the structure provided in embodiment 25.

Embodiment 153. The composition of embodiment 150, wherein the population of glycoengineered polypeptides has an N-glycan profile comprising about 30% to about 40%, about 40% to about 50%, about 50% to about 60%, about 60% to about 70%, about 70% to about 80%, about 80% to about 90%, or about 90% to about 100% of the N-glycan of the structure provided in embodiment 25 among all glycans in the N-glycan profile.

Embodiment 154. The composition of any one of embodiments 142-153, wherein the composition is a pharmaceutical composition.

Embodiment 155. The composition of embodiment 154, wherein the pharmaceutical composition comprises one or more excipients.

Embodiment 156. A Leishmania host cell expressing a glycoengineered polypeptide of any one of embodiments 1-136.

Embodiment 157. The host cell of embodiment 156, wherein the cell comprises a polynucleotide sequence encoding a glycoengineered polypeptide.

Embodiment 158. A method comprising: administering to a subject a pharmaceutical composition of embodiment 154 or 155.

Embodiment 159. The method of embodiment 158, wherein the subject has or is diagnosed as having anti-neutrophil cytoplasmic antibody (ANCA) vasculitis.

Embodiment 160. The method of embodiment 158 or 159, wherein the method is a treatment method.

Embodiment 161. The method of embodiment 158 or 159, wherein the method is a prevention method.

Embodiment 162. The method of any one of embodiments 159-161, wherein the ANCA vasculitis is Granulomatosis with Polyangiitis (GPA)/cytoplasmic ANCA (Formerly called Wegener's Granulomatosis).

Embodiment 163. The method of any one of embodiments 159-161, wherein the ANCA vasculitis is Microscopic Polyangiitis (MPA)/perinuclear ANCA.

Embodiment 164. The method of any one of embodiments 158-163, wherein the glycoengineered polypeptide is capable of simultaneously binding to the target with the first moiety and binding to an endocytic receptor-expressing cell with the second moiety, thereby causing the target to be internalized into the cell.

Embodiment 165. The method of embodiment 164, wherein internalization comprises transporting to a lysosome and/or degradation.

Embodiment 166. The method of any one of embodiments 158-165, wherein the endocytic receptor is ASGPR or a variant or fragment thereof.

Embodiment 167. The method of any one of embodiments 158-166, wherein administration of the pharmaceutical composition reduces a level of anti-neutrophil antibody as compared to a subject who has not been administered the pharmaceutical composition or as compared to the same subject prior to administration of the pharmaceutical composition.

Embodiment 168. The method of embodiment 167, wherein a reduction in the level of the anti-neutrophil autoantibody prevents neutrophil activation.

Embodiment 169. The method of any one of embodiments 158-168, wherein administration of the pharmaceutical compositions alleviates one or more symptoms of ANCA-vasculitis.

Embodiment 170. A method of treating and/or preventing Granulomatosis with Polyangiitis (GPA)/cytoplasmic ANCA (Formerly called Wegener's Granulomatosis) in a subject, the method comprising, administering to a subject a pharmaceutical composition of embodiment 154 or 155.

Embodiment 171. The method of embodiment 170, wherein the subject has an anti-PR3 autoantibody.

Embodiment 172. The method of embodiment 170 or 171, wherein administration of the pharmaceutical composition reduces a level of PR3 autoantibody as compared to a subject who has not been administered the pharmaceutical composition or as compared to the same subject prior to administration of the pharmaceutical composition.

Embodiment 173. The method of embodiment 172, wherein a reduction in the level of the anti-PR3 autoantibody prevents neutrophil activation.

Embodiment 174. A method of treating and/or preventing Microscopic Polyangiitis (MPA)/perinuclear ANCA in a subject, the method comprising, administering to a subject a pharmaceutical composition of embodiment 153 or 154.

Embodiment 175. The method of embodiment 174, wherein the subject has a MPO autoantibody.

Embodiment 176. The method of any one of embodiments 170-175, wherein administration of the pharmaceutical composition reduces a level of MPO autoantibody as compared to a subject who has not been administered the pharmaceutical composition or as compared to the same subject prior to administration of the pharmaceutical composition.

Embodiment 177. The method of embodiment 176, wherein a reduction in the level of the MPO autoantibody prevents neutrophil activation.

Embodiment 178. A method comprising,
assessing a level of an anti-neutrophil autoantibody in a sample from a subject, and
administering a pharmaceutical composition of embodiment 154 or 155;
if the level of the anti-neutrophil autoantibody is higher than a comparator.

Embodiment 179. The method of embodiment 178, wherein the comparator comprises a predetermined reference sample such as a sample obtained from an otherwise similar subject who does not have a disease or disorder, or a symptom of a disease or disorder.

Embodiment 180. The method of embodiment 178 or 179, wherein the anti-neutrophil autoantibody comprises: an anti-PR3 autoantibody or a fragment or a complex thereof; or an anti-MPO autoantibody or a fragment or a complex thereof, or a combination thereof.

Embodiment 181. The method of any one of embodiments 178-181, wherein the level of anti-neutrophil autoantibody is assessed using an ELISA.

Embodiment 182. The method of any one of embodiments 178-181, wherein the subject has or is at risk of having ANCA vasculitis.

Embodiment 183. The method of any one of embodiments 178-182, wherein the method is a treatment method.

Embodiment 184. The method of any one of embodiments 178-182, wherein the method is a prevention method.

Embodiment 185. A method treating and/or preventing ANCA vasculitis, comprising:
(a) determining a level of an anti-neutrophil autoantibody in a sample from a subject, wherein a higher level of an anti-neutrophil autoantibody in the sample as compared to a comparator indicates that the subject has or is at risk of developing ANCA vasculitis; and
(b) responsive to said determination, administering a pharmaceutical composition of embodiment 154 or 155, to the subject.

Embodiment 186. The method of embodiment 185, wherein the comparator comprises a predetermined reference sample such as a sample obtained from an otherwise similar subject who does not have ANCA vasculitis.

Embodiment 187. The method of embodiment 186, wherein the subject having a higher level of the target autoantibody is administered the pharmaceutical composition.

Embodiment 188. The method of embodiment any one of embodiments 185-187, wherein the anti-neutrophil autoantibody comprises: an anti-PR3 autoantibody or a fragment or a complex thereof; or an anti-MPO autoantibody or a fragment or a complex thereof, or a combination thereof.

Embodiment 189. The method of any one of embodiments 185-188, wherein the level of the anti-neutrophil autoantibody is assessed using an ELISA.

Embodiment 190. The method of any one of embodiments 185-189, wherein the method is a treatment method.

Embodiment 191. The method of any one of embodiments 185-189, wherein the method is a prevention method.

Embodiment 192. The method of any one of embodiments 158-191, wherein the method comprises administering the pharmaceutical composition once.

Embodiment 193. The method of any one of embodiments 158-191, wherein the method comprises administering the pharmaceutical composition repeatedly.

Embodiment 194. The method of any one of embodiments 158-193, wherein the method comprises administering the pharmaceutical composition in combination with one or more additional therapies.

Embodiment 195. The method of any one of embodiments 158-194, wherein the administration step comprises intravenous injection, intraperitoneal injection, subcutaneous injection, transdermal injection, or intramuscular injection.

Embodiment 196. The method of any one of embodiments 158-195, wherein the subject is a mammal.

Embodiment 197. The method of embodiment 196, wherein the subject is a human.

### INCORPORATION BY REFERENCE

Each publication, including scientific references to the scientific literature, patent references, and electronic databases, websites and other references accessible through the internet, are hereby incorporated by reference herein, in their entirety, for their disclosure relevant to this specification and as cited herein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A glycoengineered polypeptide comprising:
(a) a first moiety comprising one or more peptides that specifically binds to an anti-neutrophil autoantibody or a fragment or a complex thereof; and
(b) a second moiety comprising one or more glycans conjugated to the first moiety at one or more glycosylation sites wherein the anti-neutrophil autoantibody is:
(a) an anti-Proteinase 3 (PR3) autoantibody, or a fragment or a complex thereof; or
(b) an anti-Myeloperoxidase (MPO) autoantibody or a fragment or a complex thereof.

2. The glycoengineered polypeptide of claim 1, wherein the anti-PR3 autoantibody binds to PR3, or a variant or fragment thereof, optionally wherein the anti-PR3 autoantibody binds to PR3 in complex with one or more proteins.

3. The glycoengineered polypeptide of claim 1, wherein the anti-MPO autoantibody binds to MPO, or a variant or fragment thereof.

4. The glycoengineered polypeptide of any one of the preceding claims, wherein the glycoengineered polypeptide is capable of binding to:
(a) an anti-PR3 autoantibody or a fragment or a complex thereof, and
(b) an anti-MPO autoantibody or a fragment or a complex thereof.

5. The glycoengineered polypeptide of any one of the preceding claims, wherein the second moiety specifically binds to one or more endocytic receptors.

6. The glycoengineered polypeptide of claim 5, wherein the endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, DNGR1, CLEC12B, DEC-205, CLEC10, mannose 6 phosphate receptor (M6PR), or any combination thereof.

7. The glycoengineered polypeptide of any one of the preceding claims, wherein the one or more glycans comprises a terminal GlcNac, a terminal GalNac, or a terminal Gal.

8. The glycoengineered polypeptide of any one of the preceding claims, wherein the one or more glycans is an N-glycan, optionally wherein the N-glycan is linked to the first moiety of the glycoengineered polypeptide at 1, 2, 3, 4 or 5 N-glycosylation sites.

9. The glycoengineered polypeptide of any one of the preceding claims, wherein the one or more glycans comprises a glycan structure comprising GlcNAc2-Man3-GlcNAc2, GalNAc2-GlcNAc2-Man3-GlcNAc2, Gal2-GlcNAc2-Man3-GlcNAc2, GlcNAc1-Man3-GlcNAc2, Gal2-GlcNAc2-Man3-GlcNAc2, Gal1-GlcNAc2-Man3-GlcNAc2, GalNAc1-GlcNAc2-Man3-GlcNAc2, GlcNAc3-Man3-GlcNAc2, GlcNAc4-Man3-GlcNAc2, Gal3-GlcNAc3-Man3-GlcNAc2, GalNAc3-GlcNAc3-Man3-GlcNAc2, GalNAc4-GlcNAc4-Man3-GlcNAc2, Gal4-GlcNAc4-Man3-GlcNAc2, or Man-6-P—N-glycan.

10. The glycoengineered polypeptide of claim 9, wherein the glycan structure comprises a monoantennary structure, biantennary structure, a triantennary structure, or a tetraantennary structure.

11. The glycoengineered polypeptide of claim 9 or 10, wherein the glycan structure comprises a biantennary structure, optionally, wherein the glycan structure comprises a biantennary GalNAc.

12. The glycoengineered polypeptide of claim 11, wherein the biantennary GalNac binds to an asialoglycoprotein receptor (ASGPR) or a fragment or variant thereof, or a complex comprising ASGPR.

13. The glycoengineered polypeptide of any one of claims 8-12, wherein the N-glycan has a structure of: 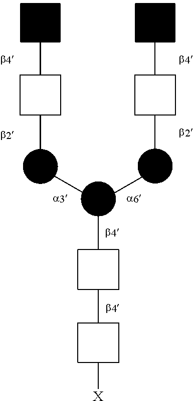
wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.

14. The glycoengineered polypeptide of any one of claims 8-13, wherein the N-glycan is conjugated to the first moiety of the glycoengineered polypeptide at at least one, two, three, or four N-glycosylation sites.

15. The glycoengineered polypeptide of any one of claims 8-14, wherein the N-glycosylation site comprises a consensus sequence of N—X—S/T or N—X—C, wherein X is any amino acid except proline.

16. The glycoengineered polypeptide of any one of claims 8-15, wherein the N-glycosylation site is naturally occurring.

17. The glycoengineered polypeptide of any one of claims 8-16, wherein the N-glycosylation site is engineered into the amino acid sequence of the first moiety.

18. The glycoengineered polypeptide of any one of claims 5-17, wherein the endocytic receptor is or comprises ASGPR or a fragment or variant thereof, optionally wherein the glycan structure of the second moiety comprises a terminal GalNac.

19. The glycoengineered polypeptide of any one of the preceding claims, wherein the glycoengineered polypeptide comprises a first moiety comprising one or more peptides that specifically binds to an anti-PR3 autoantibody or a fragment thereof.

20. The glycoengineered polypeptide of claim 19, wherein the one or more peptides that specifically bind to an anti-PR3 autoantibody are each conjugated to a second moiety.

21. The glycoengineered polypeptide of claim 19 or 20, wherein the one or more peptides that specifically bind to an anti-PR3 autoantibody is a soluble polypeptide.

22. The glycoengineered polypeptide of any one of claims 19-21, wherein the one or more peptides that specifically bind to an anti-PR3 autoantibody comprises a PR3 protein, or a fragment or a variant thereof.

23. The glycoengineered polypeptide of claim 22, wherein the PR3 protein is provided as SEQ ID NO: 1 (e.g., with or without the signal peptide) or SEQ ID NO: 40 (e.g., with or without the signal peptide) or SEQ ID NO: 45.

24. The glycoengineered polypeptide of claim 22 or 23, wherein the one or more peptides comprises at least 5% of a full length PR3 protein, or a PR3 polypeptide provided in SEQ ID NO: 1 (e.g., with or without the signal peptide) or SEQ ID NO: 40 (e.g., with or without the signal peptide) or SEQ ID NO: 45.

25. The glycoengineered polypeptide of any one of claims 19-24, wherein the fragment comprises an epitope that is recognized by a PR3 autoantibody.

26. The glycoengineered polypeptide of any one of claims 19-25, wherein the one or more polypeptides that specifically bind to an anti-PR3 autoantibody is a variant of a PR3 protein.

27. The glycoengineered polypeptide of claim 26, wherein the variant comprises a mutation at the Valine residue at position 119, the Alanine residue at position 135, the Threonine residue at position 136, or a combination thereof.

28. The glycoengineered polypeptide of claim 27, wherein the mutation is:
(a) a Valine to Isoleucine mutation;
(b) an Alanine to Threonine mutation; and/or
(c) is a Serine to Threonine mutation.

29. The glycoengineered polypeptide of any one of claims 26-28, wherein the variant comprises a mutation at one or more or all of amino acids:
(i) 71 (His), 118 (Asp) and 203 (Ser) of SEQ ID NO: 1; and/or
(ii) 180 (Phe), 181 (Phe), 228 (Leu), or 229 (Phe) of SEQ ID NO: 1.

30. The glycoengineered polypeptide of any one of claims 19-29, wherein the one or more peptides comprises a sequence having at least 85% identity to a PR3 polypeptide sequence provided in:
(i) SEQ ID NO:1 with or without the signal peptide of SEQ ID NO:6;
(ii) SEQ ID NO: 40 with or without the signal peptide SEQ ID NO: 39; or
(iii) SEQ ID NO: 45.

31. The glycoengineered polypeptide of any one of claims 19-30, wherein the one or more polypeptides that specifically bind to an anti-PR3 autoantibody comprises an antibody agent comprising an antigen binding fragment.

32. The glycoengineered polypeptide of claim 31, wherein the antibody agent comprises a full antibody, a Fab fragment, an scFv, a nanobody, duobody or a single domain antibody (e.g., a VHH).

33. The glycoengineered polypeptide of any one of claims 1-18, wherein the glycoengineered polypeptide comprises a first moiety comprising one or more peptides that specifically binds to an anti-MPO autoantibody or a fragment thereof.

34. The glycoengineered polypeptide of claim 33, wherein the one or more peptides that specifically bind to an anti-MPO autoantibody are each conjugated to a second moiety.

35. The glycoengineered polypeptide of claim 33 or 34, wherein the one or more peptides that specifically bind to an anti-MPO autoantibody is a soluble polypeptide.

36. The glycoengineered polypeptide of any one of claims 33-35, wherein the one or more peptides that specifically bind to an anti-MPO autoantibody comprise a MPO polypeptide, or a fragment or a variant thereof.

37. The glycoengineered polypeptide of claim 36, wherein the MPO polypeptide is provided as SEQ ID NO: 4 (e.g., with or without the signal peptide), SEQ ID NO: 42 (e.g., with or without the signal peptide), or SEQ ID NO: 46.

38. The glycoengineered polypeptide of claim 36 or 37, wherein the fragment comprises at least 5% of a full length MPO polypeptide, or a MPO polypeptide provided in SEQ ID NO: 4 (e.g., with or without the signal peptide), SEQ ID NO: 42 (e.g., with or without the signal peptide), or SEQ ID NO: 46.

39. The glycoengineered polypeptide of any one of claims 36-38, wherein the fragment comprises an epitope that is recognized by a MPO autoantibody.

40. The glycoengineered polypeptide of any one of claims 32-39, wherein the one or more peptides that specifically bind to an anti-MPO autoantibody is a variant of an MPO polypeptide, optionally wherein the variant is an inactive variant as compared to a wild-type MPO protein.

41. The glycoengineered polypeptide of claim 40, wherein the variant comprises a mutation at one or more or all of 261 (His), 316 (Cys), 405 (Arg) and 257 (Gln) of SEQ ID NO: 4.

42. The glycoengineered polypeptide of any one of claims 32-41, wherein the one or more peptides that specifically bind to an anti-MPO autoantibody comprises a sequence having at least 85% identity to a MPO polypeptide sequence provided in:
(i) SEQ ID NO:4 with or without the signal peptide of SEQ ID NO: 8;
(ii) SEQ ID NO: 42 with or without the signal peptide SEQ ID NO: 38; or
(iii) SEQ ID NO: 46.

43. The glycoengineered polypeptide of any one of claims 32-42, wherein the one or more peptides that specifically bind to an anti-MPO autoantibody comprise an antibody agent comprising an antigen binding fragment.

44. The glycoengineered polypeptide of claim 43, wherein the antibody agent comprises a full antibody, a Fab fragment, an scFv, a nanobody, a duobody, or a single domain antibody (e.g., a VHH).

45. The glycoengineered polypeptide of any one of the preceding claims, wherein the first moiety comprises (i) one or more anti-PR3 autoantibody binding polypeptides and (ii) one or more anti-MPO autoantibody binding polypeptides.

46. The glycoengineered polypeptide of any one of the preceding claims, wherein the polypeptide comprises one or more additional elements chosen from:
(a) a linker,
(b) a spacer,
(c) a cleavage peptide, e.g., an IRES or a protease cleavage site,
(d) a signal peptide,
(e) a tag, e.g., a cleavable tag,
(f) a half-life extender domain, e.g., an Fc domain or albumin,
(g) any combination of (a)-(f).

47. The glycoengineered polypeptide of any one of the preceding claims, wherein the second moiety is conjugated to the first moiety in vivo.

48. The glycoengineered polypeptide of claim 47, wherein the conjugation occurs in a cell, optionally wherein the cell is a Leishmania cell.

49. The glycoengineered polypeptide of any one of claims 1-46, wherein the second moiety is conjugated to the first moiety by chemical conjugation, optionally wherein chemical conjugation comprises click chemistry.

50. A polynucleotide encoding the glycoengineered polypeptide of any one of the preceding claims.

51. A composition comprising a glycoengineered polypeptide of any one of claims 1-50.

52. A composition comprising a population of glycoengineered polypeptides of any one of claims 1-50, wherein the population of glycoengineered polypeptides has an N-glycan profile that is at least 30% homogeneous at one or more of the N-glycosylation site(s).

53. The composition of claim 52, wherein the N-glycan profile comprises about 30% of the N-glycan of the structure provided in claim 13.

54. The composition of claim 52 or 53, wherein the composition is a pharmaceutical composition.

55. A Leishmania host cell expressing a glycoengineered polypeptide of any one of claims 1-50, wherein the cell comprises a polynucleotide sequence encoding a glycoengineered polypeptide.

56. A method comprising:
administering to a subject a pharmaceutical composition of claim 54.

57. The method of claim 56, wherein the subject has or is diagnosed as having anti-neutrophil cytoplasmic antibody (ANCA) vasculitis.

58. The method of claim 56 or 57, wherein the method is a treatment method or a prevention method.

59. The method of 56 or 57, wherein the ANCA vasculitis is Granulomatosis with Polyangiitis (GPA)/cytoplasmic ANCA (Formerly called Wegener's Granulomatosis).

60. The method of any one of claims 56-59, wherein when administered to a subject the glycoengineered polypeptide is capable of simultaneously binding to the target with the first moiety and binding to an endocytic receptor-expressing cell with the second moiety, thereby causing the target to be internalized into the cell.

61. The method of claim 60, wherein internalization comprises transporting to a lysosome and/or degradation.

62. The method of claim 60 or 61, wherein the endocytic receptor is ASGPR or a variant or fragment thereof.

63. The method of any one of claims 56-62, wherein administration of the pharmaceutical composition reduces a level of anti-neutrophil antibody as compared to a subject who has not been administered the pharmaceutical composition or as compared to the same subject prior to administration of the pharmaceutical composition.

64. The method of claim 63, wherein a reduction in the level of the anti-neutrophil autoantibody prevents neutrophil activation.

65. The method of any one of claims 56-64, wherein administration of the pharmaceutical compositions alleviates one or more symptoms of ANCA-vasculitis.

66. A method of treating and/or preventing Granulomatosis with Polyangiitis (GPA)/cytoplasmic ANCA (Formerly called Wegener's Granulomatosis) in a subject, the method comprising, administering to a subject a pharmaceutical composition of claim 54, wherein the subject has an anti-PR3 autoantibody.

67. The method of claim 66, wherein administration of the pharmaceutical composition reduces a level of PR3 autoantibody as compared to a subject who has not been administered the pharmaceutical composition or as compared to the same subject prior to administration of the pharmaceutical composition.

68. A method of treating and/or preventing Microscopic Polyangiitis (MPA)/perinuclear ANCA in a subject, the method comprising, administering to a subject a pharmaceutical composition of claim 54, wherein the subject has an MPO autoantibody.

69. The method of claim 68, wherein administration of the pharmaceutical composition reduces a level of MPO autoantibody as compared to a subject who has not been administered the pharmaceutical composition or as compared to the same subject prior to administration of the pharmaceutical composition.

70. A method comprising,
assessing a level of an anti-neutrophil autoantibody in a sample from a subject, and
administering a pharmaceutical composition of claim 54;
if the level of the anti-neutrophil autoantibody is higher than a comparator.

71. The method of any one of claims 56-70, wherein the administration step comprises intravenous injection, intraperitoneal injection, subcutaneous injection, transdermal injection, or intramuscular injection.

72. The method of any one of claims 56-71, wherein the subject is a mammal.
